(19) United States
(12) Patent Application Publication
(10) Pub. No.: US 2010/0330089 A1
(43) Pub. Date: Dec. 30, 2010

(54) ANTI-CD20 THERAPEUTIC COMPOSITIONS AND METHODS

(75) Inventors: Nitin K. Damle, Upper Saddle River, NJ (US); Lioudmila Tchistiakova, Andover, MA (US); Kyriaki Dunussi-Joannopoulos, Belmont, MA (US); Sandy Alexander Simon, Seattle, WA (US); William Brady, Bothell, WA (US); Laura Sue Grosmaire, Hobart, WA (US); Jeffrey Alan Ledbetter, Shoreline, WA (US)

Correspondence Address: ROPES & GRAY LLP, PATENT DOCKETING 39/361, 1211 AVENUE OF THE AMERICAS, NEW YORK, NY, 10036-8704, US

(73) Assignees: Wyeth, Madison, NJ (US); Trubion Pharmaceuticals Inc., Seattle, WA (US)

(21) Appl. No.: 12/664,166

(22) PCT Filed: Jun. 12, 2008
(86) PCT No.: PCT/US08/07464

§ 371 Date: Sep. 13, 2010

Related U.S. Application Data
(60) Provisional application No. 60/934,341, filed on Jun. 12, 2007.

(51) Int. Cl. A61K 39/395; C07K 16/28; C07H 21/00; C12N 15/63; C12N 5/10; C12P 21/02; G01N 33/566; A61P 37/02; A61P 29/00; A61P 25/00; A61P 35/00

(52) U.S. Cl. 424/135.1; 530/387.3; 424/133.1; 536/23.53; 435/320.1; 435/328; 435/69.6; 435/7.24

(57) ABSTRACT

The present invention provides materials and methods for treatment of diseases involving aberrant B-cell activity, using a CD20-specific binding molecule, in particular, antibodies or antigen binding fragment thereof. The compositions disclosed herein is useful for the treatment and diagnosis of B-cell disorders, such as B-cell malignancies and autoimmune diseases.

### FIELD OF THE INVENTION

The invention provides materials and methods for treatment of diseases involving aberrant B-cell activity, using a CD20-specific binding molecule. The compositions disclosed herein are useful for the treatment and diagnosis of B-cell disorders, such as B-cell malignancies and autoimmune diseases.

### BACKGROUND INFORMATION

In its usual role, the human immune system protects the body from damage from foreign substances and pathogens. One way in which the immune system protects the body is by production of specialized cells called B lymphocytes or B-cells. B-cells produce antibodies that bind to, and in some cases mediate destruction of, a foreign substance or pathogen.

In some instances though, the human immune system, and specifically the B lymphocytes of the human immune system, go awry and disease results. There are numerous cancers that involve uncontrolled proliferation of B-cells. There are also numerous autoimmune diseases that involve B-cell production of antibodies that, instead of binding to foreign substances and pathogens, bind to parts of the body. In addition, there are numerous autoimmune and inflammatory diseases that involve B-cells in their pathology, for example, through inappropriate B-cell antigen presentation to T-cells or through other pathways involving B-cells. For example, autoimmune-prone mice deficient in B-cells do not develop autoimmune kidney disease, vasculitis or autoantibodies. (Shlomchik et al., J Exp. Med. 1994, 180:1295-306). Interestingly, these autoimmune-prone mice that possess B-cells but are deficient in immunoglobulin production, do develop autoimmune diseases when induced experimentally (Chan et al., J Exp. Med. 1999, 189:1639-48), indicating that B-cells play an integral role in development of autoimmune disease.

B-cells can be identified by molecules on their cell surface. CD20 was the first human B-cell lineage-specific surface molecule identified by a monoclonal antibody. It is a non-glycosylated, hydrophobic 35 kDa B-cell transmembrane phosphoprotein that has both its amino and carboxy ends situated inside the cell. Einfeld et al., EMBO J. 1988, 7:711-17. CD20 is expressed by all normal mature B-cells, but is not expressed by precursor B-cells or plasma cells. Natural ligands for CD20 have not been identified, and the function of CD20 in B-cell biology is still incompletely understood.

Certain anti-CD20 monoclonal antibodies can affect the viability and growth of B-cells. (Clark et al., Proc. Natl. Acad. Sci. USA 1986, 83:4494-98). Extensive cross-linking of CD20 can induce apoptosis in B lymphoma cell lines (Shan et al., Blood 1998, 91:1644-52), and cross-linking of CD20 on the cell surface has been reported to increase the magnitude and enhance the kinetics of signal transduction, for example, as detected by measuring tyrosine phosphorylation of cellular substrates. (Deans et al., J. Immunol. 1993, 146:846-53). Therefore, in addition to cellular depletion by complement and ADCC mechanisms, Fc-receptor binding by certain CD20 monoclonal antibodies in vivo may promote apoptosis of malignant B-cells by CD20 cross-linking, consistent with the theory that effectiveness of CD20 therapy of human lymphoma in a SCID mouse model may be dependent upon Fc-receptor binding by the CD20 monoclonal antibody (Funakoshi et al., J. Immunotherapy 1996, 19:93-101). The presence of multiple membrane spanning domains in the CD20 polypeptide (Einfeld et al., EMBO J. 1988, 7:711-17; Stamenkovic et al., J. Exp. Med. 1988, 167:1975-80; Tedder et al., J. Immunol. 1988, 141:4388-4394), prevent CD20 internalization after antibody binding, and this was recognized as an important feature for therapy of B-cell malignancies when a murine CD20 monoclonal antibody, 1F5, was injected into patients with B-cell lymphoma, resulting in significant depletion of malignant cells and partial clinical responses (Press et al., Blood 1987, 69:584-91).

Because normal mature B-cells also express CD20, normal B-cells are depleted by anti-CD20 antibody therapy (Reff et al., Blood 1994, 83:435-445). After treatment is completed, however, normal B-cells can be regenerated from CD20 negative B-cell precursors; therefore, patients treated with anti-CD20 therapy do not experience significant immunosuppression.

CD20 is expressed by malignant cells of B-cell origin, including B-cell lymphoma and chronic lymphocytic leukemia (CLL). CD20 is not expressed by malignancies of pre-B-cells, such as acute lymphoblastic leukemia. CD20 is therefore a good target for therapy of B-cell lymphoma, CLL, and other diseases in which B-cells are involved in the disease etiology. Other B-cell disorders include autoimmune diseases in which autoantibodies are produced during the differentiation of B-cells into plasma cells.

The use of monoclonal antibodies (mAb) targeting molecules preferentially expressed on the surface of a tumor cell is now a well-established therapeutic strategy, with at least 7 distinct mAb therapeutics approved and currently being used in cancer therapy: Rituxan® (RITUXAN), trastuzumab, alemtuzumab, cetuximab, panitumumab, bevacizumab and gemtuzumab ozogamicin. Such mAb therapeutics mediate their anti-tumor activity via two distinct mechanisms. The first mechanism involves the mAb-mediated inhibition of the key receptor-ligand/counter-receptor interactions that contribute to the tumor growth and the second mechanism is dependent on the participation of the effector components of the host's immune system such as FcR⁺ effector cells capable of mediating antibody-dependent cellular cytotoxicity (ADCC) and humoral factors such as complement capable of mediating complement-dependent cytotoxicity (CDC). In case of the latter, the mAb therapeutic must possess the capability to interact with the FcγRs on effector cells and complement. With the exception of EGFR-targeted panitumumab, each of the five immunotherapeutic mAb currently being used in cancer therapy possess the capability to engage the effector components of the immune system.

RITUXAN was the first mAb to be approved for clinical use in cancer. RITUXAN is a recombinant mouse human IgG1 chimeric mAb in which variable domains of the heavy and light chains of a murine anti-CD20 mAb were fused to the human constant regions of IgG1.

In addition, CD20 has also been targeted by radioimmunotherapeutic agents to treat B-cell related diseases. One treatment consists of anti-CD20 antibodies prepared in the form of radionuclides for treating B-cell lymphoma (e.g., ¹³¹I-labeled anti-CD20 antibody), as well as a ⁸⁹Sr-labeled form for the palliation of bone pain caused by prostate and breast cancer metastases (Endo, Gan To Kagaku Ryoho 1999, 26: 744-748).

In one study, RITUXAN was tested for safety, tolerability and preliminary clinical efficacy for the treatment of 18 patients with Systemic Lupus Erythematosus (SLE) (who were non-immunosuppressed patients). Of the 18 patients treated, six patients received one infusion of RITUXAN at 100 mg/m² (low dose), six patients received one infusion of RITUXAN at 375 mg/m² (medium dose), and six patients received four weekly infusions of RITUXAN at 375 mg/m² (high dose). Even at the low or medium dosage, three of the 12 patients (25%) developed elevated levels of human anti-chimera antibodies (HACA) at two months.

Accordingly, there is a need to develop novel CD20-specific binding molecules for therapy, preferably, novel CD20-specific binding molecules that do not cause, or have a reduced potential to cause, a HACA reaction when administered to patients who are not immunosuppressed. In addition, although there has been extensive research carried out on antibody-based therapies, there remains a need in the art for compositions and methods to treat diseases associated with aberrant B-cell activity.

### SUMMARY OF THE INVENTION

The invention relates to novel CD20 binding molecules that are useful in the diagnosis and treatment of B cell mediated diseases and conditions in a subject in need thereof including but not limited to B cell cancers, rheumatoid arthritis and lupus erythematosis. In various embodiments, the invention provides novel heavy chain CDR sequences, light chain CDR sequences, novel variable domain sequences comprising the CDR sequences and CD20 binding molecules comprising a novel CDR or variable domain sequence, nucleic acids, vectors, host cells, compositions and kits comprising the CDRs, binding domains or molecules comprising them. In some embodiments, the CD20 binding molecules comprising a novel CDR sequence or variable domain is an antibody or an antigen-binding fragment thereof. In other embodiments, the CD20 binding molecule is a small modular immunopharmaceutical SMIP. In some embodiments, the antibody or SMIP is humanized and comprises human sequence framework and constant region sequences. The CD20 binding molecules of the invention bind CD20 on cell, show CDC and ADCC activity, deplete the CD19+ B cell in blood, bone marrow and lymph nodes, reduce B cell lymphoma tumor growth, and/or reduce the progression and effects of disseminated lymphoma. The CD20 binding molecules of the invention also are useful to detect and quantify the presence of CD20 or cells expressing them, for example in a biological sample from a subject.

### BRIEF DESCRIPTION OF THE DRAWINGS

FIG. 1. Binding of anti-CD20 SMIPs to primary B cells. Primary B cells isolated from buffy coat were incubated with the indicated concentration of a variety of anti-CD20 SMIPs, as specified. Binding of SMIPs was analyzed by flow cytometry (MFI) using labeled anti-human IgG-PE antibody (Fc specific). Ec50 of each SMIP was calculated accordingly.

FIG. 2. In Vitro Growth Inhibitory Effect of 018011 (also referred to as 18011 or 011, which are the identical molecule) against Human B-Cell Lymphomas.

FIG. 3A-3B. Complement dependent Cytotoxicity assay of anti-CD20 SMIPs. A: Ramos B cells were incubated with anti-CD20 SMIPs in the presence of 10% human sera (Quidel) for 3.5 hours at 37° C. Cell death was measured by LDH release from cells (Promega kit). B: primary human B cells (5×10⁵) isolated from buffy coat were pre-incubated with anti-human CD55 antibody (2 μg/ml) for 10 min at 37° C. Anti-CD20 SMIPs at the indicated concentrations and serum (10%) were then added. After 3.5-hour incubation, cell death was assessed by 7-AAD staining and flow cytometry analysis.

FIG. 4A-4B. These in vitro studies demonstrate that 018011 binds in a dose-dependent manner to CD20+ B-cell lymphoma cells and is able to bring about both Fc-mediated cellular cytotoxicity as well as complement-dependent cytotoxicity against CD20+ B-cell lymphoma target cells. This demonstration of effector functional capability may be important to the ability of 018011 to inhibit growth of human B-cell lymphomas. A. Complement-Dependent Cytotoxicity of 018011 Against SU-DHL4 B-Cell Lymphoma Cells. B. Complement-Dependent Cytotoxicity of 018011 Against BJAB B-Cell Lymphoma Cells.

FIG. 5. Antibody dependent cytotoxicity assay of anti-CD20 SMIPs. BJAB lymphoma cells were labeled with CFSE, then incubated with SMIPs and activated human PBMC. Cells were stained with PI and analyzed by flow cytometry. Only CFSE⁺ cells were assessed for cell death.

FIG. 6A-6B. A. Fc-mediated Cellular Cytotoxicity of 018011 Against SU-DHL4 B-cell Lymphoma. B. Fc-mediated Cellular Cytotoxicity of 018011 Against Ramos B-cell Lymphoma.

FIG. 7. ADCC of CD20-SMIPs against Ramos B cells.

FIG. 8. Depletion of peripheral CD19+ B Cells in non-human primate. Both 2LM 20-4 and 2LM 20-4 mut Fc demonstrated effective depletion of peripheral CD19+ B cells in non-human primate that is at least comparable to Rituxan.

FIG. 9. Depletion of bone marrow CD19+ B Cells in non-human primate. Both 2LM 20-4 and 2LM 20-4 mut Fc demonstrated effective depletion of bone marrow CD19+ B cells in non-human primate that is at least comparable to Rituxan.

FIG. 10. Depletion of Lymph Node CD19+ B Cells in non-human primate. Both 2LM 20-4 and 2LM 20-4 mut Fc demonstrated effective depletion of lymph node CD19+ B cells in non-human primate that is at least comparable to Rituxan.

FIG. 11. Pharmacokinetic (PK) analysis of 2LM 20-4 and 2LM 20-4 mut Fc in cynomolgus monkeys after IV administration. PK profiles of 2LM 20-4 and 2LM 20-4 mut Fc are comparable to that of Rituxan.

FIG. 12. Effect of 018011 and Rituximab on Ramos Subcutaneous Xenografts Established in Balb/c Nude Mice.

FIG. 13. Survival Analysis of 018011 and Rituximab on Ramos Subcutaneous Xenografts Established in Balb/c Nude Mice.

FIG. 14. Effect of 018011, TRU-015 and Rituximab on Ramos Subcutaneous Xenografts Established in nu/nu Nude Mice.

FIG. 15. Survival Analysis of 018011, TRU-015 and Rituximab on Ramos Subcutaneous Xenografts Established in nu/nu Nude Mice.

FIG. 16. Survival Analysis of 018011, TRU-015- and Rituximab-treated Scid Mice with Disseminated Ramos B Cell Lymphoma.

FIG. 17. Detection of Human B Lymphoma Cells in Bone Marrow of Scid Mice with Disseminated Disease.

FIG. 18. Survival Analysis of 018011-, TRU-015-, and Rituximab-treated Scid Mice with Disseminated WSU-DLCL2 Diffuse Large Cell Lymphoma.

FIG. 19. Effect of 018011 Administered either Intravenously or Intraperitoneally on the Growth of Ramos Subcutaneous Xenografts.

FIG. 20. Effect of 018011 and Rituximab on Non-irradiated and Irradiated Ramos Subcutaneous Xenografts.

FIG. 21A-21C. Mean Tumor Volumes Over Time. On study Day 0, nude mice bearing palpable Ramos tumors were sorted into treatment groups (n=8/group) such that the mean tumor volume for each group was equivalent. Mice were treated IV on days 0, 2, 4, 6, and 8 with 100 μg of human IgG, TRU-015, 018008 (also referred to as 18008 or 008, which are the identical molecule), 018011, or 2Lm20-4. Tumors were measured on the indicated days with a caliper and tumor volume was calculated using the formula: V=½[length×(width)²]. Once an animal was taken out of the study due to tumor volume exceeding specified limits, the value for the last tumor volume was carried forward. Results are shown only through day 10, when the last control mice were sacrificed.

FIG. 22A-22C. Tumor Volumes of Individual Mice at an Early Time Point. Mice were treated and monitored, and tumor volumes were determined as described in the legend to FIG. 21. Results are shown in terms of tumor volume of individual mice on day 8 (the last time point in which all mice were alive) (i) or relative tumor volume of individual mice on day 8 relative to day 0 (ii). Significant differences among groups were determined using a one-way ANOVA with Dunnett's multiple comparison post test (for comparison with huIgG treated controls) and Tukey's multiple comparison post test (for all other pairwise comparisons); p values for all pair wise comparisons are indicated.

FIG. 23A-23C. Survival Percentages of Mice Treated with TRU-015 or HuCD20 SMIPs. Mice were treated and monitored, and tumor volumes were determined as described in the legend to FIG. 21. Tumor volumes were determined at least 3 times a week (M W F) with the exception that monitoring was switched to once per week during time periods when all mice remaining in the study had no palpable tumors. Mice were sacrificed when tumor volumes reached more than 1500 mm³ (or 1200 mm³ on Fridays). No mice were found dead or sacrificed for other reasons.

FIG. 24A-24C. Percentage of Tumor-Free Mice Over Time. Mice were treated and monitored, and tumor volumes were determined as described in the legend to FIG. 21. A mouse was considered “tumor-free” if it had no palpable tumor. Tumor volumes were determined at least 3 times a week (M W F) with the exception that monitoring was switched to once per week during time periods when all mice remaining in the study had no palpable tumors.

FIG. 25A-25C. Mean Body Weights of Mice Over Time. Mice were treated and monitored as described in the legend to FIG. 21. Body weights were determined on the indicated days of the study.

FIG. 26. Flow Cytometric Evaluation of 018011 or Rituximab Bound to Cells Isolated from Ramos B-cell Lymphoma Xenografts.

FIG. 27. In Vitro Growth Inhibitory Effect of 018011 against Human B-Cell Lymphomas.

FIG. 28. Effect of Cross-linking of 018011 on Activation-induced Death of Ramos B-Cell Lymphoma Cells.

FIG. 29 is a table summarizing amino acid residues at several key positions of the exemplary humanized SMIPs. Residues listed under the “Hinge” column depict the residues for positions 220, 226, 229 and 230 of SEQ ID NO: 60. Residue 331 in the “IgG1 Fc” column refers to residue 331 in SEQ ID NO: 61.

### DETAILED DESCRIPTION OF THE INVENTION

### I. Definitions

In order that the present invention may be more readily understood, certain terms are first defined. Additional definitions are set forth throughout the detailed description.

A “CD20 binding molecule” according to various embodiments of the invention is a molecule comprising a CD20-binding portion of the humanized CD20 binding molecule specifically exemplified herein. A type of CD20 binding molecule contemplated by the invention is an antibody or a CD20-binding fragment thereof. A binding molecule may be modified according to methods standard in the art, for example, to improve its binding affinity, to improve its specificity, to diminish its immunogenicity, to alter its effector functions and/or to improve its availability in the body of an individual. Such modifications may include, for example, amino acid sequence modifications or expression as a fusion protein. Such fusion proteins are also binding molecules according to the invention. An exemplary binding molecule of the invention is a small modular immunopharmaceutical (SMIP).

The term “antibody” refers to an immunoglobulin or fragment thereof, and encompasses any such polypeptide comprising an antigen-binding fragment of an antibody. The term includes but is not limited to polyclonal, monoclonal, monospecific, polyspecific, humanized, human, single-chain, chimeric, synthetic, recombinant, hybrid, mutated, grafted, and in vitro generated antibodies.

The term “antibody” also includes antigen-binding fragments of an antibody. Examples of antigen-binding fragments include, but are not limited to, Fab fragments (consisting of the V_{L}, V_{H}, C_{L} and C_{H}1 domains); Fd fragments (consisting of the V_{H} and C_{H}1 domains); Fv fragments (referring to a dimer of one heavy and one light chain variable domain in tight, non-covalent association); dAb fragments (consisting of a V_{H} domain); isolated CDR regions; (Fab′)₂ fragments, bivalent fragments (comprising two Fab fragments linked by a disulphide bridge at the hinge region), scFv (referring to a fusion of the V_{L} and V_{H} domains, linked together with a short linker), and other antibody fragments that retain antigen-binding function. The part of the antigen that is specifically recognized and bound by the antibody is referred to as the “epitope.”

An antigen-binding fragment of an anti-CD20 antibody of the invention can be produced by conventional biochemical techniques, such as enzyme cleavage, or recombinant DNA techniques known in the art. These fragments may be produced by proteolytic cleavage of intact antibodies by methods well known in the art, or by inserting stop codons at the desired locations in the vectors using site-directed mutagenesis, such as after C_{H}1 to produce Fab fragments or after the hinge region to produce (Fab)₂ fragments. For example, Papain digestion of antibodies produces two identical antigen-binding fragments, called “Fab” fragments, each with a single antigen-binding site, and a residual “Fc” fragment. Pepsin treatment of an antibody yields an F(ab′)₂ fragment that has two antigen-combining sites and is still capable of cross-linking antigen. Single chain antibodies may be produced by joining V_{L} and V_{H} coding regions with a DNA that encodes a peptide linker connecting the V_{L} and V_{H} protein fragments

“In vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term excludes sequences generated by genomic rearrangement in an immune cell.

An antigen-binding fragment/domain may comprise an antibody light chain variable region (V₁) and an antibody heavy chain variable region (V_{H}); however, it does not have to comprise both. Fd fragments, for example, have two V_{H} regions and often retain some antigen-binding function of the intact antigen-binding domain. Examples of antigen-binding fragments of an antibody include (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region; (3) a Fd fragment having the two V_{H} and C_{H}1 domains; (4) a Fv fragment having the V_{L} and V_{H} domains of a single arm of an antibody, (5) a dAb fragment (Ward et al., (1989) Nature 341:544-546), that has a V_{H} domain; (6) an isolated complementarity determining region (CDR), and (7) a single chain Fv (scFv). Although the two domains of the Fv fragment, V_{L} and V_{H}, are coded for by separate genes, they can be joined, using recombinant DNA methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are evaluated for function in the same manner as are intact antibodies.

The term “human antibody” includes antibodies having variable and constant regions corresponding substantially to human germline immunoglobulin sequences known in the art, including, for example, those described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo). The human antibody can have at least one, two, three, four, five, or more positions replaced with an amino acid residue that is not encoded by the human germline immunoglobulin sequence. CDRs are as defined by Kabat or in Chothia C, Lesk A M, Canonical structures for the hypervariable regions of immunoglobulins, J Mol Biol. 1987 Aug. 20; 196(4):901-17. CDRs typically refer to regions that are hypervariable in sequence and/or form structurally defined loops, for example, Kabat CDRs are based on sequence variability, as described in Sequences of Proteins of Immunological Interest, US Department of Health and Human Services (1991), eds. Kabat et al, or alternatively, to the location of the hypervariable structural loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modelling software, which defines the contact hypervariable regions based on crystal structure. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops.

The term “effective amount” refers to a dosage or amount that is sufficient over a course of therapy to reduce any CD20 activity, to ameliorate one or more clinical symptoms or achieve a desired biological outcome.

The phrase “inhibit” or “antagonize” CD20 activity and its cognates refer to a reduction, inhibition, or otherwise diminution of at least one activity of CD20 due to binding a CD20-specific binding molecule, wherein the reduction is relative to the activity of CD20 in the absence of the same molecule. Inhibition or antagonism does not necessarily indicate a total elimination of the CD20 biological activity. A reduction in activity may be at least about 10%, 20%, 30%, 40%, 50%, 60%, 70%, 80%, 90%, or more.

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions; or at least 70% (w/w) pure; or at least 80% (w/w) pure; or at least 90% pure; or at least 95%, 96%, 97%, 98%, 99%, or 100% (w/w) pure.

The term “therapeutic agent” is a substance that treats or assists in treating a medical disorder. Therapeutic agents may include, but are not limited to, anti-proliferative agents, anti-cancer agents including chemotherapeutics, anti-virals, anti-infectives, immune modulators, and the like that modulate immune cells or immune responses in a manner that complements the reduction of the CD20 activity by the CD20-specific binding molecules of the invention. Non-limiting examples and uses of therapeutic agents are described herein.

As used herein, a “therapeutically effective amount” of a CD20-specific binding molecule refers to an amount of a CD20-specific binding molecule that is effective, upon single or multiple dose administration to a subject (such as a human patient) at treating, preventing, curing, delaying, reducing the severity of, and/or ameliorating at least one symptom of a disorder or recurring disorder, or prolonging the survival of the subject beyond that expected in the absence of treatment. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously. The invention specifically contemplates that one or more CD20-specific binding molecules may be administered according to methods of the invention, each in an effective dose.

The term “treatment” or ‘treating” refers to a therapeutic, preventative or prophylactic measures. A therapeutic treatment may improve at least one symptom of disease in an individual receiving treatment or may delay worsening of a progressive disease in a individual, or prevent onset of additional associated diseases. The treatment may be administered to a subject having a medical disorder or who ultimately may acquire the disorder, to prevent, cure, delay, reduce the severity of, and/or ameliorate one or more symptoms of the disorder or recurring disorder, or in order to prolong the survival of a subject beyond that expected in the absence of treatment.

The term “human CD20” is intended to refer to the human B lymphocyte-restricted differentiation antigen (also known as Bp35). CD20 is expressed during early pre-B cell development and remains until plasma cell differentiation. The CD20 molecule may regulate a step in the activation process which is required for cell cycle initiation and differentiation, and is usually expressed at very high levels on neoplastic B cells. CD20 is present on both “normal” B cell as well as “malignant” B cells (i.e. those B cells whose unabated proliferation can lead to B cell lymphoma).

### II. CD20-Specific Binding Molecules

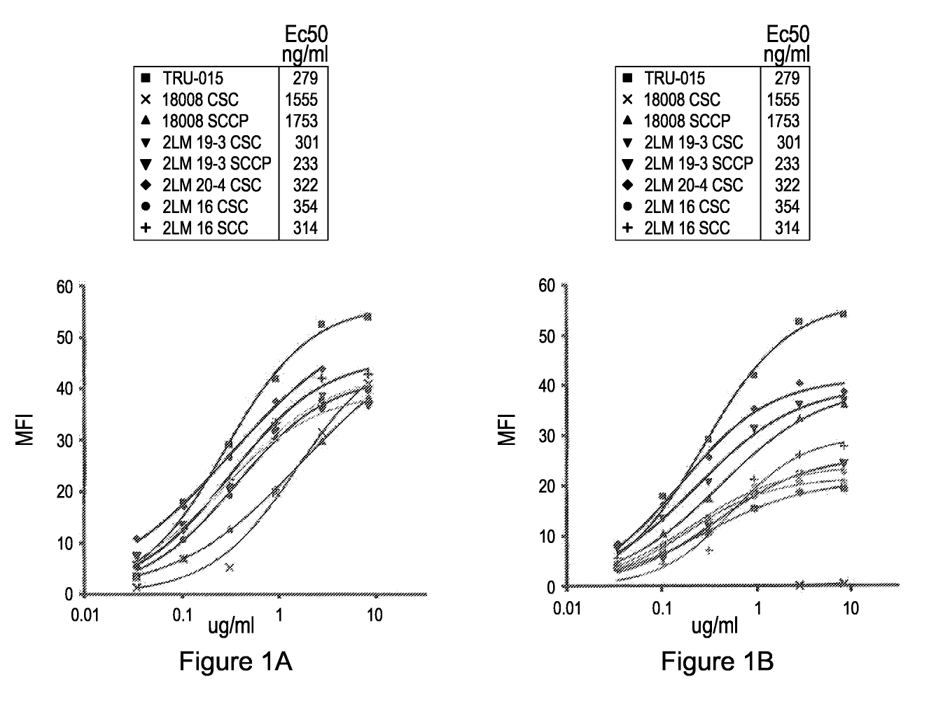

According to the invention, the CD20 binding molecules bind CD20 on primary B cells and on B cell lymphoma cell lines including NU-DHL1, Ramos, SU-DHL4, SU-DHL5, and WSU-DLCL2. In various embodiments, the CD20 binding molecules bind CD20 expressing cells with an EC50 comparable to the EC50 shown in FIG. 1 and/or bind in a dose-dependent manner. In various embodiments, the CD20 binding molecules possess one or more properties selected from: complement mediated cytotoxicity (CDC) in primary B cells and in at least Ramos, SU-DHL4 and BJAB cells, in some cases with at least 40% cytotoxicity; antibody-dependent (or Fc-dependent cyotoxicity (ADCC or FcCC) in at least Ramos, BJAB and SU-DHL4 cells, that is dose-dependent and in some cases with at least 40% cytotoxicity; maintains CD19+ B cell depletion in bone marrow and lymph nodes for a longer duration than RITUXAN® or for longer than 8 days and for at least 10, 12, 14, 16, 18, 20 or 21 days; reduces the growth established xenograft tumors in a mouse model as described in the Examples and is protective when administered in the early stages of disseminated lymphoma in various mouse models, as described in the Examples.

In some embodiments, the α-CD20 binding molecules are Small Modular-ImmunoPharmaceuticals (SMIPs). In one aspect, the invention provides humanized CD20-specific binding molecules. Applicants have provided 57 novel CD20-specific binding molecules that are useful for treatment of mammalian subjects, including humans and domestic animals. The invention also provides nucleic acid sequences encoding CD20-specific binding molecules.

In certain embodiments, the CD20-specific binding molecules of the invention have a high binding affinity, a low dissociation rate, and specifically bind to human CD20.

In certain embodiments, the humanized CD20-specific binding molecules of the invention are antibodies, in particular anti-CD20 monoclonal antibodies (mAbs), or antigen-binding fragments thereof.

A chimeric antibody is an antibody having portions derived from different antibodies. For example, a chimeric antibody may have a variable region and a constant region derived from two different antibodies. The donor antibodies may be from the same or different species. In certain embodiments, the variable region of a chimeric anti-CD20 antibody of the invention is non-human, e.g., murine, (or a combination of non-human and human) and the constant region is human.

The humanized CD20 binding molecules of the invention also include CDR grafted humanized anti-CD20 antibodies. In one embodiment, the humanized antibody comprises heavy and/or light chain CDRs of a humanized anti-CD20 SMIP of the invention (SEQ ID NOS: 1-59) and heavy chain and light chain frameworks and constant regions of a human acceptor immunoglobulin. Methods of making humanized antibodies are disclosed in U.S. Pat. Nos. 5,530,101; 5,585,089; 5,693,761; 5,693,762; and 6,180,370 each of which is incorporated herein by reference in its entirety.

Antigen-binding fragments of the antibodies of the invention, that retain the binding specificity of the intact antibody, are also included in the invention. Antigen-binding fragments include partial or full heavy chains or light chains, variable regions, or CDR regions of any CD20-specific antibodies described herein.

In some embodiments, a humanized CD20-specific antibody of the invention comprises the heavy chain CDR3, the light chain CDR3, or both, of any binding molecule exemplified in the sequence listing of the application. In embodiments comprising a HCDR3 and an LCDR3, they can be from the same molecule or different molecules in the sequence listing of the application. In some embodiments, a humanized CD20-specific antibody of the invention comprises the heavy chain CDR1, CDR2 and CDR3, the light chain CDR1, CDR2 and CDR3, or all six CDRs of a binding molecule exemplified in the sequence listing of the application or the HCDR1-3 and LCDR1-3 may be from different binding molecules in the sequence listing of the application. In some embodiments, a humanized CD20-specific antibody of the invention is an antibody comprising a heavy chain variable domain, a light chain variable domain or both, of a binding molecule exemplified in the sequence listing of the application. Also encompassed by the invention are humanized anti-CD20 antibodies comprising any V_{H} shown in the sequence listing of the application, any V_{L} shown in the sequence listing of the application, any combination of such V_{H} and V_{L} or any V_{H}/V_{L} pair shown in the sequence listing of the application, or antigen binding portion of such antibodies.

In certain embodiments, a CD20-specific binding molecule of the invention is a CD20-specific, small modular immunopharmaceutical (SMIP).

A humanized anti-CD20 SMIP of the invention contains three modular domains: a binding domain, a hinge domain, and an effector domain. In some embodiments, the binding domain of an anti-CD20 SMIP of the invention comprises a V_{H} domain and a V_{L} domain. The hinge domain of an anti-CD20 SMIP of the invention performs two complementary functions by providing a flexible link between the binding domain and effector domain while also controlling association, or multimerization, of the SMIPs. The effector domain of a humanized anti-CD20 SMIP of the invention can comprise e.g., an human antibody Fc domain, or a non-antibody protein with effector function. The effector domain determines which type of immune cell is activated and regulates the balance of effector functions employed, including the relative activity of antibody dependent cellular cytotoxicity (ADCC) or complement dependent cytotoxicity (CDC). Effector domains also can be engineered to regulate SMIP drug multimerization.

The binding domain of a SMIP of the invention may have one or more binding regions, such as variable light chain and variable heavy chain binding regions derived from one or more immunoglobulins. These regions are typically separated by linker peptides, which may be any linker peptide known in the art to be compatible with domain or region joinder. Exemplary linkers are linkers based on the Gly₄Ser linker motif, such as (Gly₄Ser)n, where n=1-5 or any linker shown in the sequence listing of the application. Any suitable linker can be used in the context of the present invention, examples of which are described in WO 2007/146968.

In some embodiments, the binding domain comprises a humanized single chain immunoglobulin-derived Fv product, which may include all or a portion of at least one immunoglobulin light chain variable region and all or a portion of at least one immunoglobulin heavy chain variable region, joined by a linker.

The hinge domain of a humanized anti-CD20 SMIP of the invention may be a naturally occurring peptide, a mutated or genetically engineered peptide, or an artificial peptide. For example, the hinge region may be derived from an immunoglobulin hinge region (e.g., portions of the immunoglobulin heavy chain sequence that is responsible for forming intra-chain immunoglobulin-domain disulfide bonds in C_{H}1 and C_{H}2 regions). The hinge region may also may be a fragment of (e.g., 5-65 amino acids, 10-50 amino acids, 15-35 amino acids, 18-32 amino acids, 20-30 amino acids, 21, 22, 23, 24, 25, 26, 27, 28 or 29 amino acids) an immunoglobulin polypeptide chain region classically regarded as having hinge function. The hinge domain may also include amino acids located (according to structural criteria for assigning a particular residue to a particular domain that may vary, as known in the art) in an adjoining immunoglobulin domain, such as the C_{H}1 domain, the C_{H}2 domain, or the variable domain.

The hinge domain of a humanized anti-CD20 SMIP of the invention may be a human hinge region, i.e., the hinge region of the heavy chain of a human antibody. In embodiments comprising a human hinge region, the hinge region may be from any human immunoglobulin isotype, such a human IgG immunoglobulin (i.e., a human IgG1, IgG2, IgG3, or IgG4). In certain embodiments, the hinge domain comprises zero or one cysteine residue. In some embodiments, a humanized anti-CD20 SMIP of the invention comprises a hinge shown in any one of SEQ IDS 60 or 63 to 66.

A humanized anti-CD20 SMIP of the invention contains sufficient amino acid sequence of a constant region of an immunoglobulin to provide an effector function, preferably ADCC and/or CDC. For example, the effector domain may comprise the sequence of a human immunoglobulin C_{H}2 domain, or may comprise human immunoglobulin C_{H}2 and C_{H}3 domains. In embodiments comprising C_{H}2 and C_{H}3 domains, the domains may be from the same or different human immunoglobulins. In other embodiments, the effector domain may comprise the sequences of a human immunoglobulin IgE C_{H}3 and C_{H}4 regions.

In certain embodiments, the CD20-binding domain of the SMIPs of the invention comprises a V_{H} amino acid sequence, a V_{L} amino acid sequence, any of both a V_{H} and a V_{L} amino acid sequence or a V_{H}/V_{L} pair set forth in SEQ ID NOs: 1-59. In certain embodiments, the hinge domain of the SMIPs of the invention comprises an amino acid sequence as set forth in SEQ ID NO: 60. In certain embodiments, the effector domain of the SMIPs of the invention comprises an amino acid sequence as set forth in SEQ ID NO: 60 or 61.

In certain embodiments, the CD20-binding domain of the SMIPs of the invention comprises a V_{H} region with human immunoglobulin framework sequences and the heavy chain CDR1, CDR2, and CDR3 amino acid sequences light chain CDR1, CDR2, and CDR3 amino acid sequences, or both, of the CD20-binding domain of a SMIP selected from SEQ ID NOs: 1-59. In certain embodiments, the SMIP hinge domain further comprises SEQ ID NO: 60, and the effector domain comprises SEQ ID NO: 61.

The invention provides numerous heavy chain V_{H}, light chain V_{L}, and CDR sequences useful for generating CD20-specific binding molecules. For example, one or more of the CDRs shown in the sequence listing of the application can be combined with a human framework sub-region (e.g., a fully human FR1, FR2, FR3, or FR4) to generate a CD20-specific binding molecule. In certain embodiments, the CD20-specific binding molecules of the invention comprise three CDRs from a light chain variable region, and three CDRs from a heavy chain variable region, wherein the heavy chain CDRs and the light chain CDRs are from the same reference sequence. The CDRs of the invention may be grafted to any type of immunoglobulin frameworks.

The invention also provides humanized CD20-specific binding molecules comprising an amino acid sequence that is substantially identical or substantially homologous to a sequence shown in the sequence listing of the application, and humanized CD20-specific binding molecules comprising CDRs that are substantially identical or substantially homologous to the CDR sequences (underlined) shown in The sequence listing of the application. For example, a number of amino acids or nucleotide bases may be changed in the sequences shown in the sequence listing of the application, in particular in one or more CDRs, framework regions, or both. Accordingly, in some embodiments, a CD20-specific binding molecule of the invention has an amino acid sequence that is at least 80% identical to a sequence as set forth in SEQ ID NOs 1-59. In other embodiments, the amino acid sequence is 85%, 90%, 95%, 96%, 97%, 98% o 99% identical to a sequence the sequence of as set forth in SEQ ID NOs 1-59. Any suitable linker can be used in the context of the present invention, examples of which are described in WO 2007/146968.

Sequences substantially identical or homologous (e.g., at least about 85% sequence identity) to the sequences disclosed herein are also part of this application. In some embodiment, the sequence identity can be about 85%, 90%, 95%, 96%, 97%, 98%, 99% or higher. Alternatively, in connection with nucleic acids, substantial identity or homology exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Changes to the amino acid sequence may be generated by changing the nucleic acid sequence encoding the amino acid sequence. A nucleic acid sequence encoding a variant of a given CDR may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR, all of which are techniques well known in the art. For example, site-directed mutagenesis may be used to prepare substitution variants (see, e.g., Carter et al., (1985) Nucleic Acids Res. 13: 4431-4443 and Kunkel et. al., (1987) Proc. Natl. Acad. Sci. USA 82: 488-492, both of which are hereby incorporated by reference).

In another aspect, the invention provides nucleic acids encoding an anti-CD20 binding molecule of the invention. In some embodiments, the nucleic acid encodes a polypeptide comprising the V_{H} or V_{L}, amino acid sequence set forth in any one of SEQ ID NOS: 1-59, or encodes a polypeptide comprising the amino acid sequence of any one of SEQ ID NOS: 1-59. In some embodiments, the nucleic acid is any one of SEQ ID NOS: 67-126 or a fragment of one of those sequences encoding a V_{H} region or a V_{L} region.

### III. Producing CD20 Binding Molecules

The humanized CD20-specific binding molecules of the invention can be prepared, for example, by recombinant DNA technologies. For example, in the case of an antibody, or an antigen-binding fragment thereof of the invention, a host cell may be transfected with one or more recombinant expression vectors carrying DNA fragments encoding the immunoglobulin light and heavy chains of the antibody, or an antigen-binding fragment of the antibody, such that the light and heavy chains are expressed in the host cell and, preferably, secreted into the medium in which the host cell is cultured, from which medium the antibody can be recovered. In the case of a SMIP, a nucleic acid encoding the SMIP is introduced into and expressed in a host cell.

Standard recombinant DNA methodologies may be used to obtain antibody heavy and light chain genes or a nucleic acid encoding the SMIP, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

For example, to express an antibody, or an antigen-binding fragment thereof, of the invention, nucleic acids encoding the light and heavy chain variable regions may be first obtained. These nucleic acids can be obtained by amplification and modification of human germline light and heavy chain variable region genes using the polymerase chain reaction (PCR). Germline DNA sequences for human heavy and light chain variable region genes are known in the art. Once the V_{H} and V_{L} fragments are obtained, these sequences can be genetically engineered to encode, for example, one or more of the SMIP sequences, the V_{H} and the V_{L} fragments of the SMIP sequences, or the CDRs of the SMIP sequences disclosed herein (see, e.g., the sequence listing of the application).

To express the antibodies of the invention or antigen-binding fragments thereof, one or more nucleic acids encoding partial or full-length light and heavy chains or in the case of SMIPs, a nucleic acid encoding the SMIP may be inserted into an expression vector or vectors such that the nucleic acids are operably linked to transcriptional and translational control sequences. The expression vector and expression control sequences are generally chosen to be compatible with the expression host cell used.

In addition to the antibody heavy chain and/or light chain genes, the recombinant expression vectors of the invention may additionally carry regulatory sequences that control the expression of the antibody chain(s) (or fragments) or SMIPs in a host cell, such as promoters, enhancers or other expression control elements (e.g., polyadenylation signals) that control the transcription or translation of the nucleic acid(s) encoding the binding molecule of the invention. Such regulatory sequences are known in the art (see, e.g., Goeddel, Gene Expression Technology: Methods in Enzymology 185, Academic Press, San Diego, Calif. (1990), herein incorporated by reference). It will be appreciated by those skilled in the art that the design of the expression vector, including the selection of regulatory sequences may depend on such factors as the choice of the host cell to be transformed, the level of expression of protein desired, etc. Exemplary regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to sequences encoding the antibody heavy chain and/or light chain or SMIP of the invention and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Other suitable selectable marker genes include the dihydrofolate reductase (DHFR) gene (for use in dhfr-host cells with methotrexate selection/amplification) and the neomycin gene (for G418 selection).

For expression, the expression vector(s) encoding the antibody heavy and light chains or the SMIP may be transfected into a host cell by standard techniques, such as electroporation, calcium-phosphate precipitation, or DEAE-dextran transfection. In certain embodiments, the expression vector used to express the CD20-specific binding molecules of the invention are viral vectors, such as retro-viral vectors. Such viral vectors may also be used to generate stable cell lines (as a source of a continuous supply of the CD20-specific binding molecules).

Suitable mammalian host cells for expressing the recombinant humanized anti-CD20 binding molecules of the invention include PER.C6 cells (Crucell, The Netherlands), Chinese Hamster Ovary (CHO cells) (including dhfr-CHO cells, described in Urlaub and Chasin, (1980) Proc. Natl. Acad. Sci. USA 77: 4216-4220, used with a DHFR selectable marker, e.g., as described in R. J. Kaufman and P. A. Sharp (1982) Mol. Biol. 159: 601-621), NS0 myeloma cells, COS cells and SP2 cells. Additionally, host cells express GnTin (described in WO9954342 and U.S. Pat. Pub. 20030003097, both herein incorporated by reference) may also be used, such that expressed CD20-specific binding molecules have increased ADCC activity.

When recombinant expression vectors encoding the antibody or SMIP are introduced into mammalian host cells, the antibodies and SMIPs are generally produced by culturing the host cells for a period of time sufficient to allow for expression of the protein in the host cells or, more preferably, secretion of the antibody or antigen-binding fragment or the SMIP into the culture medium in which the host cells are grown. The anti-CD20 binding molecule can be recovered from the culture medium using standard protein purification methods.

It will be understood that variations on the above procedure are within the scope of the present invention. For example, recombinant DNA technology may be used to remove some or all of the DNA encoding either or both of the light and heavy chains that is not necessary for binding to CD20. In addition, bi-functional antibodies may be produced in which one heavy and one light chain are an antibody of the invention and the other heavy and light chain are specific for an antigen other than CD20 (e.g., by crosslinking an antibody of the invention to a second antibody by standard chemical crosslinking methods).

Methods for making SMIPs have been described in U.S. Patent Publication Nos. 2003/0133939, 2003/0118592, and 2005/0136049, which are incorporated herein by reference in their entirety. In certain embodiments, SMIPs of the invention are binding domain-immunoglobulin fusion proteins that feature (1) a binding domain for a cognate structure (such as an antigen, a counterreceptor or the like), (2) an IgG1, IGA or IgE hinge region or a mutant IgG1 hinge region having either zero, one or two cysteine residues, and (3) immunoglobulin C_{H}2 and C_{H}3 domains. In certain embodiments, the binding domain comprises one or two cysteine (Cys) residues in the hinge region. In certain embodiments, when the binding domain comprises two Cys residues, the first Cys, which is involved in binding between the heavy chain and light chain, is not deleted or substituted with another amino acid.

The humanized anti-CD20 SMIPs of the invention are related to a chimeric anti-CD20 SMIP, TRU-015, which is a recombinant (murine/human) single chain protein that binds to the CD20 antigen, as described in US 2007/0213293. The binding domain of TRU-015 was based on the publically available 2H7 binding domais. The binding domain is connected to the effector domain, the C_{H}2 and C_{H}3 domains of human IgG1 through a modified CSS hinge region.

### IV. Therapeutic Uses

In another aspect, the invention provides a method of treating a subject having or suspected of having a disease associated with aberrant B-cell activity, comprising administering to a patient a therapeutically effective amount of a humanized CD20-specific binding molecule of the invention. In one embodiment, the CD20-specific binding molecule is a CD20-specific small, modular immunopharmaceutical (SMIP).

“Aberrant B-cell activity” refers to cell activity that deviates from the normal, proper, or expected course. For example, aberrant cell activity may include inappropriate proliferation of cells whose DNA or other cellular components have become damaged or defective. Aberrant B-cell activity may include cell proliferation whose characteristics are associated with a disease caused by, mediated by, or resulting in inappropriately high levels of cell division, inappropriately low levels of apoptosis, or both. Such diseases may be characterized, for example, by single or multiple local abnormal proliferations of cells, groups of cells or tissue(s), whether cancerous or non-cancerous, benign or malignant, described more fully below. Aberrant B-cell activity may also include aberrant antibody production, such as production of autoantibodies, or overproduction of antibodies typically desirable at normal levels. It is contemplated that aberrant B-cell activity may occur in certain subpopulations of B-cells and not in other subpopulations. Aberrant B-cell activity may also include inappropriate stimulation of T-cells, such as by inappropriate B-cell antigen presentation to T-cells or by other pathways involving B-cells.

“A subject having or suspected of having a disease associated with aberrant B-cell activity” is a subject in which a disease or a symptom of a disease may be caused by aberrant B-cell activity, may be exacerbated by aberrant B-cell activity, or may be relieved by regulation of B-cell activity. Examples of such diseases are B cell cancers (such as B-cell lymphoma, a B-cell leukemia, a B-cell myeloma), a disease characterized by autoantibody production or a disease characterized by inappropriate T-cell stimulation, such as by inappropriate B-cell antigen presentation to T-cells or by other pathways involving B-cells.

In one aspect, an individual treated by methods of the invention demonstrates an improved response to treatment with the CD20-specific binding molecule described herein, which is improved over the response to other treatments, such as for example, ENBREL® (Amgen/Wyeth), HUMIRA® (Abbott), REMICADE® (Johnson & Johnson/Schering-Plough), RITUXAN® (Genentech/Roche), ocrelizumab (Genentech/Roche), ORENCIA® (BMS), ACTEMRA® (Roche/Chugai), CIMZIA® (UCB Pharma) for treatment of RA and RITUXAN® (Genentech/Roche), ocrelizumab (Genentech/Roche), belimumab (HGS), epratuzumab (Immunomedics/UCB), Humax CD20® (Genmab/GSK), atacicept (Zymogenetics), ORENCIA® (BMS), and ACTEMRA® (Roche/Chugai) for treatment of lupus (SLE). A response that is improved over other treatments refers to a clinical response wherein treatment by the method of the invention results in a clinical response in a patient that is better than the other therapy, either alone or in combination with other agents. An improved response is assessed by comparison of clinical criteria well-known in the art and described herein. Exemplary criteria include, but are not limited to, duration of B cell depletion, reduction in B cell numbers overall, reduction in B cell numbers in a biological sample, reduction in tumor size, reduction in the number of tumors existing and/or appearing after treatment, and improved overall response as assessed by patients themselves and physicians, e.g., using an International Prognostic Index. The improvement may be in one or more than one of the clinical criteria. An improved response with the method of the invention may be due to an inadequate response to previous or current treatment, for example, because of toxicity (e.g., infusion related adverse events) and/or inadequate efficacy of the other treatment. In addition, there may be dosing regimen or schedules of the present inventive binding molecules that are improved (e.g., subcutaneous administration).

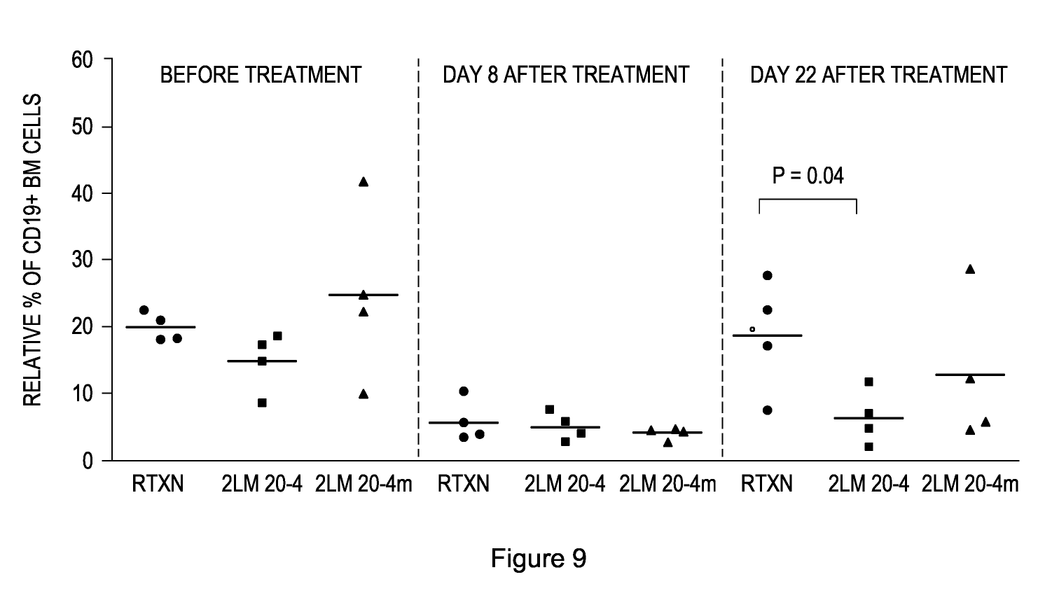

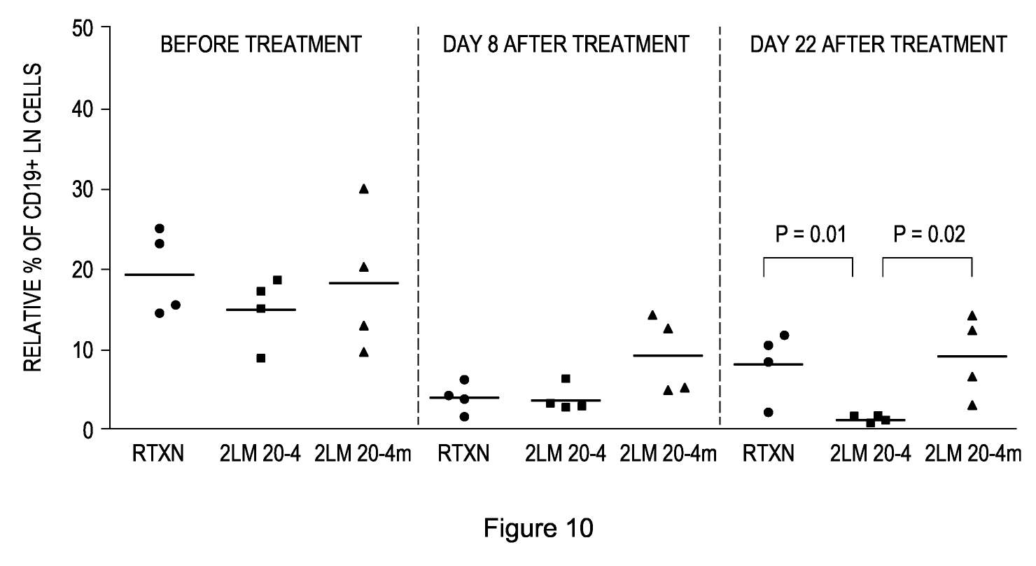

For example, treatment with a humanized CD20 SMIP produced a significant reduction in CD19+ B cells in the bone marrow and lymph nodes twenty-two days after treatment with the humanized CD20 SMIP compared to RITUXAN®. See FIGS. 9 and 10 and Example 4.

In rheumatoid arthritis, major cell types responsible for chronic inflammation and subsequent cartilage destruction and bone erosion in the joints are macrophages, synovial fibroblasts, neutrophils, and lymphocytes (Marrack et al., Nat Med. 2001; 7:899-905). It has been demonstrated that T and B lymphocytes that infiltrate inflamed synovial tissues are often organized into structures that resemble lymphoid follicles (Berek & Kim, Semin Immunol. 1997; 9:261-268; Berek &, Schroder, Ann N Y Acad Sci. 1997; 815:211-217; Kim & Berek, Arthritis Res. 2000; 2:126-131). Molecular analysis of B cells isolated from synovial follicular structures during rheumatoid arthritis demonstrated the importance of B cells in local antigen-driven specific immune responses and in increased production of rheumatoid factor (RF), the high-affinity antibodies with self-reactivity (Weyand & Goronzy, Ann N Y Acad Sci. 2003; 987:140-149; Gause et al, BioDrugs. 2001; 15:73-79). Positivity for RF is associated with more aggressive articular disease and a higher frequency of extra-articular manifestations (van Zeben et al., Ann Rheum Dis. 1992; 51:1029-1035)

Evidence regarding the pathogenicity of B cells in RA has been recently obtained from clinical trials in patients with refractory disease by using B cell ablation with rituximab (Rituxan®), a human chimeric anti-CD20 monoclonal antibody (Leandro et al, Ann Rheum Dis. 2002; 61:883-888; Edwards et al., N Engl J Med. 2004; 350:2572-2581). In all groups treated with rituximab, a significantly higher proportion of patients had a 20 percent improvement in disease symptoms according to the ACR criteria. All ACR responses were maintained at week 48 in the rituximab-methotrexate group. In this study involving 161 patients with active RA, serious infections occurred in one patient (2.5 percent) in the control group and in four patients (3.3 percent) in the rituximab groups, indicating that B cell depletion is a relatively safe therapy in RA.

CD20 is a 35 KD non-glycosylated tetraspanning cell membrane-embedded phosphoprotein, is restricted to the B-cell lineage and is expressed on pre-B cells, immature B cells, mature naive and memory B cells, but not on early pro-B cells and plasma cells. At present, the mechanism by which removal of pathogenic B cells and their precursors, but not antibody-secreting plasma cells, leads to clinical improvement remains elusive (Cragg et al., Therapy. Curr Dir Autoimmun 2005; 8:140-17410). Given that B cells exist as lymphoid aggregates within the synovium of RA patients, B cell functions other than antibody production (e.g., cytokine production, antigen presentation, provision of costimulatory signals to T cells) might also play an important role in disease pathogenesis (Martin & Chan, Immunity. 2004; 20:517-527).

In vitro studies suggest that rituximab induces lysis of CD20-positive lymphoma cells through three possible mechanisms: ADCC, CDC and direct signaling leading to apoptosis (Clark & Ledbetter, Ann Rheum Dis 2005; 64:77-8012). Evidence in humans suggests that B cell lysis in SLE occurs likely via ADCC and apoptosis by engagement of FcγRIIIa (CD16) on natural killer cells and macrophages, since the degree of B cell depletion in SLE as well as the clinical response of lymphoma, depends upon FcγRIIIa polymorphism (Anolik et al., Arthritis Rheum 2003; 48:455-459).

Anti-CD20 therapy has also been tested in several other autoimmune disorders, including systemic lupus erythematosus (SLE), providing in the process novel insights into the role of B cells in autoimmunity (Eisenberg R., Arthritis Res Ther. 2003; 5:157-159). A majority of SLE patients receiving rituximab demonstrate complete B cell depletion and clinical improvement (Anolik et al., Curr Rheumatol Rep. 2003; 5:350-356). In contrast, those without B cell depletion do not respond to the treatment. The B cell number is usually lowest 1-3 months after the initial dose and this depletion lasts for 3-12 months.

Patients may develop human antichimeric antibody (HACA) after treatment, as rituximab is a chimeric antibody. In one study, 33% of patients treated with rituximab had high HACA titers (Looney et al., Curr Opin Rheumatol. 2004; 16:180-185). Interestingly, these include patients who received only a single dose of rituximab. The HACAs are found to be associated with less effective B cell depletion, and lower serum levels of rituximab at 2 months after initial infusion.

Rituximab treatment induces an almost complete depletion of all peripheral blood B cell populations in patients with RA (Leandro et al., Arthritis Rheum 2006; 54:613-620). Failure to achieve 97% depletion of circulating B cells for at least 3 months in 1 patient was associated with a lack of response to treatment. B cell repopulation of the peripheral blood was dependent upon the formation of naïve B cells, rather than the expansion of memory B cells. Patients who experienced an earlier disease relapse, at the time of B cell return, tended to show a higher number of circulating memory B cells at repopulation, as compared with patients whose relapse occurred later. Less extensive B cell depletion in solid tissues thus may be associated with an earlier relapse of RA.

The recent clinical successes of rituximab in autoimmunity indicate that there is need for the development of alternative B cell-targeting therapeutics that would be less immunogenic and could potentially delete all clonal remnants of pathologic autoreactive B cells in the hope of reestablishing immune tolerance.

Despite significant clinical responses seen in patients treated with existing CD20-targeted therapies, the therapeutic effects of these agents are not durable, and the frequency of disease relapse, particularly in lymphoma patients, remains high. CD20-targeted therapies are highly effective at depleting both normal and transformed B cells from circulation. However, their ability to ablate B cells embedded in tissues or in the lymphatics is more limited, possibly due to the reduced ability of large biomolecules to access these sites or to increased target cell resistance provided by the tissue microenvironment. Whatever the reason, these environments may provide a protected reservoir for surviving B cells or lymphoma cells that can reemerge subsequent to an apparently successful treatment regimen and give rise to disease relapse.

Treatment of non-human primates with the humanized SMIP followed by resection and analysis of tissues has demonstrated a superior capacity of the CD20 SMIP to eliminate tissue embedded B cells compared to control treatments. This, combined with a clear capacity to eliminate B cells from circulation, suggest that patients treated with this agent will respond favorably in the near term to effective B cell ablation, but moreover that efficacy following treatment will be of significantly greater duration as compared to current CD20-targeted therapies. For these agents, it appears that the depth of depletion in bone marrow and lymph node tissue is significant, indicating that these molecules may be able to deplete B cells efficiently in immunologically active, relevant areas. Thus, these molecules may provide a preferred therapy to treat a broader set of patients, including those in whom there is a need to enhance such efficacy.

In a related aspect, the individual treated by the methods of the invention is also administered RITUXAN. In one embodiment, RITUXAN may have been administered as a first line of treatment and continue when treatment with a method of the invention has begun. In another embodiment, RITUXAN treatment is discontinued after treatment with a method of the invention has begun.

“A subject having or suspected of having a rheumatic disease” is a subject or individual affected by a disease or disorder of articular origin or of the musculoskeletal system, affecting such areas as joints, cartilage, muscles, nerves, and tendons. It is further contemplated that the subject having or suspected of having a rheumatic disease may have previously received therapy to treat a rheumatic disease. In one embodiment, the rheumatic disease includes, but is not limited to, rheumatoid arthritis, ankylosing spondylitis, dermatomyositis, Henoch Schonlein purpura, juvenile rheumatoid arthritis, psoriatic arthritis, Raynaud's syndrome, Reiter's syndrome, sarcoidosis, spondyloarthropathies, progressive systemic sclerosis and myositis.

“A subject having or suspected of having a central nervous system autoimmune disease” or “central nervous system disorder” is a subject or individual affected by a disease or disorder affecting the central nervous system, including the brain and spinal cord, or such areas as the optic nerve. It is further contemplated that subject having or suspected a central nervous system disorder may have previously received therapy to treat a central nervous system disorder. In one embodiment, the central nervous system autoimmune disease includes, but is not limited to, multiple sclerosis, allergic encephalomyelitis, neuromyelitis optica, lupus myelitis and lupus cerebritis.

“Vasculitis” refers to a disease or disorder associated with inflammation in a blood vessel. Exemplary vasculitis disorders include, but are not limited to, Behcet's disease, central nervous system vasculitis, Churg-Strauss syndrome, cryoglobulinemia, giant cell arteritis, Henoch Schonlein purpura, hypersensitivity vasculitis/angiitis, Kawasaki disease, leucocytoclastic vasculitis, polyantitis, polyarteritis nodosa, polymyalgia, polychondritis, rheumatoid vasculitis, Takayasu's arteritis, Wegener's granulamatosis, vasculitis due to hepatitis, familial Mediterranean fever, microscopic polyangiitis, Cogan's syndrome, Whiskott-Aldrich syndrome and thromboangiitis obliterans.

Methods contemplated by the invention are useful for treating diseases such as B cell cancers (for example, B-cell lymphomas, B-cell leukemias, B-cell lymphomas), diseases characterized by autoantibody production, or diseases characterized by inappropriate T-cells stimulation of T-cells, such as by inappropriate B-cell antigen to T-cells or by other pathways involving B-cells.

B-cell cancers include B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia and chronic myoblastic leukemia) and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

Disorders characterized by autoantibody production are often considered autoimmune diseases. Autoimmune diseases include, but are not limited to: arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, mysathenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), pemphigoid bullous, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans.

Rheumatoid arthritis (RA) is a chronic disease characterized by inflammation of the joints, leading to swelling, pain, and loss of function. Patients having RA for an extended period usually exhibit progressive joint destruction, deformity, disability and even premature death.

Systemic Lupus Erythematosus (SLE) is an autoimmune disease caused by recurrent injuries to blood vessels in multiple organs, including the kidney, skin, and joints. In patients with SLE, a faulty interaction between T cells and B-cells results in the production of autoantibodies that attack the cell nucleus. There is general agreement that autoantibodies are responsible for at least some aspects of SLE. It is contemplated that new therapies that deplete the B-cell lineage, allowing the immune system to reset as new B-cells are generated from precursors, would offer hope for long lasting benefit in SLE patients.

Crohn's disease and a related disease, ulcerative colitis, are the two main disease categories that belong to a group of illnesses called inflammatory bowel disease (IBD). Crohn's disease is a chronic disorder that causes inflammation of the digestive or gastrointestinal (GI) tract. Although it can involve any area of the GI tract from the mouth to the anus, it most commonly affects the small intestine and/or colon. In ulcerative colitis, the GI involvement is limited to the colon. Multiple sclerosis (MS) is also an autoimmune disease. It is characterized by inflammation of the central nervous system and destruction of myelin, which insulates nerve cell fibers in the brain, spinal cord, and body. Although the cause of MS is unknown, it is widely believed that autoimmune T cells are primary contributors to the pathogenesis of the disease. However, high levels of antibodies are present in the cerebral spinal fluid of patients with MS, and some theories predict that the B-cell response leading to antibody production is important for mediating the disease. The course of MS is difficult to predict, and the disease may at times either lie dormant or progress steadily. Several subtypes, or patterns of progression, have been described, which are relevant not only for prognosis but also for therapeutic decisions. Relapsing-remitting describes the initial course of 85% to 90% of individuals with MS. This subtype is characterized by unpredictable attacks (relapses) followed by periods of months to years of relative quiet (remission) with no new signs of disease activity. Deficits suffered during the attacks may either resolve or may be permanent. When deficits always resolve between attacks, this is referred to as “benign” MS. Secondary progressive describes around 80% of those with initial relapsing-remitting MS, who then begin to have neurologic decline between their acute attacks without any definite periods of remission. This decline may include new neurologic symptoms, worsening cognitive function, or other deficits. Secondary progressive is the most common type of MS and causes the greatest amount of disability. Primary progressive describes the approximately 10% of individuals who never have remission after their initial MS symptoms. Decline occurs continuously without clear attacks. The primary progressive subtype tends to affect people who are older at disease onset. Progressive relapsing describes those individuals who, from the onset of their MS, have a steady neurologic decline but also suffer superimposed attacks; and is the least common of all subtypes.

Crohn's disease may be characterized by antibodies against neutrophil antigens, i.e., the “perinuclear anti-neutrophil antibody” (pANCA), and Saccharomyces cervisiae, i.e. the “anti-Saccharomyces cervisiae antibody” (ASCA). Many patients with ulcerative colitis have the pANCA antibody in their blood, but not the ASCA antibody, while many Crohn's patients exhibit ASCA antibodies, and not pANCA antibodies. One method of evaluating Crohn's disease is using the Crohn's disease Activity Index (CDAI), based on 18 predictor variables scores collected by physicians. CDAI values of 150 and below are associated with quiescent disease; values above that indicate active disease, and values above 450 are seen with extremely severe disease (Best, et al., “Development of a Crohn's disease activity index.” Gastroenterology 70:439-444, 1976. However, since the original study, some researchers use a ‘subjective value’ of 200 to 250 as an healthy score.

Autoimmune thyroid disease results from the production of autoantibodies that either stimulate the thyroid to cause hyperthyroidism (Graves' disease) or destroy the thyroid to cause hypothyroidism (Hashimoto's thyroiditis). Stimulation of the thyroid is caused by autoantibodies that bind and activate the thyroid stimulating hormone (TSH) receptor. Destruction of the thyroid is caused by autoantibodies that react with other thyroid antigens.

Sjogren's syndrome is an autoimmune disease characterized by destruction of the body's moisture-producing glands.

Immune thrombocytopenic purpura (ITP) is caused by autoantibodies that bind to blood platelets and cause their destruction.

Myasthenia Gravis (MG) is a chronic autoimmune neuromuscular disorder characterized by autoantibodies that bind to acetylcholine receptors expressed at neuromuscular junctions leading to weakness of the voluntary muscle groups.

Psoriasis, is characterized by autoimmune inflammation in the skin and also associated with arthritis in 30% of cases.

Also contemplated is the treatment of idiopathic inflammatory myopathy (IIM), including dermatomyositis (DM) and polymyositis (PM). Inflammatory myopathies have been categorized using a number of classification schemes. Miller's classification schema (Miller, Rheum Dis Clin North Am. 1994, 20:811-826) identifies 2 idiopathic inflammatory myopathies (IIM), polymyositis (PM) and dermatomyositis (DM).

Polymyositis and dermatomyositis are chronic, debilitating inflammatory diseases that involve muscle and, in the case of DM, skin. These disorders are rare, with a reported annual incidence of approximately 5 to 10 cases per million adults and 0.6 to 3.2 cases per million children per year in the United States (Targoff, Curr Probl Dermatol. 1991, 3:131-180). Idiopathic inflammatory myopathy is associated with significant morbidity and mortality, with up to half of affected adults noted to have suffered significant impairment (Gottdiener et al., Am J. Cardiol. 1978, 41:1141-49). Miller (Rheum Dis Clin North Am. 1994, 20:811-826 and Arthritis and Allied Conditions, Ch. 75, Eds. Koopman and Moreland, Lippincott Williams and Wilkins, 2005) sets out five groups of criteria used to diagnose IIM, i.e., Idiopathic Inflammatory Myopathy Criteria (IIMC) assessment, including muscle weakness, muscle biopsy evidence of degeneration, elevation of serum levels of muscle-associated enzymes, electromagnetic triad of myopathy, evidence of rashes in dermatomyositis, and also includes evidence of autoantibodies as a secondary criteria.

IIM associated factors, including muscle-associated enzymes and autoantibodies include, but are not limited to, creatine kinase (CK), lactate dehydrogenase, aldolase, C-reactive protein, aspartate aminotransferase (AST), alanine aminotransferase (ALT), and antinuclear autoantibody (ANA), myositis-specific antibodies (MSA), and antibody to extractable nuclear antigens.

### V. Pharmaceutical Compositions and Methods of Administration

In another aspect of the invention, a CD20-specific binding molecule of the invention is administered as a pharmaceutical composition. To administer a CD20-specific binding molecule to humans or test animals, it is preferable to formulate the binding molecule in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like.

In addition, compounds may form solvates with water or common organic solvents. Such solvates are contemplated as well.

The CD20-specific binding molecule compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient. In certain embodiments, injection, especially intravenous, are preferred.

Pharmaceutical compositions of the present invention containing a CD20-specific binding molecule used in a method of the invention may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

Formulation of the pharmaceutical composition will vary according to the route of administration selected (e.g., solution, emulsion). For example, an appropriate composition comprising an anti-CD20 antibody (or the CD20-binding fragment thereof, such as a SMIP) to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers

A variety of aqueous carriers, e.g., water, buffered water, 0.4% saline, 0.3% glycine, or aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The CD20-specific binding molecule composition can be lyophilized for storage and reconstituted in a suitable carrier prior to use. This technique has been shown to be effective with conventional immunoglobulins. Any suitable lyophilization and reconstitution techniques can be employed. It will be appreciated by those skilled in the art that lyophilization and reconstitution can lead to varying degrees of antibody activity loss and that use levels may have to be adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

The concentration of CD20-specific binding molecule in these formulations can vary widely, for example from less than about 0.5%, usually at or at least about 1% to as much as 15 or 20% by weight and will be selected primarily based on fluid volumes, viscosities, etc., in accordance with the particular mode of administration selected. Thus, a typical pharmaceutical composition for parenteral injection could be made up to contain 1 ml sterile buffered water, and 50 mg of antibody. A typical composition for intravenous infusion could be made up to contain 250 ml of sterile Ringer's solution, and 150 mg of antibody. Actual methods for preparing parenterally administrable compositions will be known or apparent to those skilled in the art and are described in more detail in, for example, Remington's Pharmaceutical Science, 15th ed., Mack Publishing Company, Easton, Pa. (1980). An effective dosage of antibody is within the range of 0.01 mg to 1000 mg per kg of body weight per administration.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In all cases the form must be sterile and must be fluid to the extent that easy syringability exists. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

Compositions useful for administration may be formulated with uptake or absorption enhancers to increase their efficacy. Such enhancers include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

In addition, the properties of hydrophilicity and hydrophobicity of the compositions contemplated for use in the invention are well balanced, thereby enhancing their utility for both in vitro and especially in vivo uses, while other compositions lacking such balance are of substantially less utility. Specifically, compositions contemplated for use in the invention have an appropriate degree of solubility in aqueous media which permit absorption and bioavailability in the body, while also having a degree of solubility in lipids which permits the compounds to traverse the cell membrane to a putative site of action. Thus, antibody compositions contemplated are maximally effective when they can be delivered to the site of target antigen activity.

In one aspect, methods of the invention include a step of administration of a pharmaceutical composition comprising a CD20-specific binding molecule of the invention.

Methods of the invention are performed using any medically-accepted means for introducing a therapeutic directly or indirectly into a mammalian subject, including but not limited to injections, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection, epidural, and or surgical implantation at a particular site is contemplated as well. Pharmaceutical compositions for oral or transmucosal administration may be either in liquid or solid composition form.

In certain embodiments, a pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Solutions or suspensions used for subcutaneous application typically include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetra acetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic. In some embodiments, the CD20 binding molecule is in lyophilized dosage form and may further comprise the one or more of following excipients: L-histidine, L-methionine, sucrose, and polysorbate 80.

Also contemplated is subcutaneous therapy using a pharmaceutical composition of the present invention. These therapies can be administered daily, weekly, or, more preferably, biweekly, or monthly. Ultimately the attending physician will decide on the appropriate duration of intravenous. or subcutaneous therapy, or therapy with a small molecule, and the timing of administration of the therapy, using the pharmaceutical composition of the present invention.

In an exemplary embodiment, to supply a high dosage subcutaneously, in which the volume limitation is small (e.g., about 1-1.2 ml per injection), the concentration of CD20 binding molecule is generally 100 mg/ml or greater.

In other embodiments, administration is performed at the site of a cancer or affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations of the invention implanted near the cancer.

Therapeutic compositions may also be delivered to the patient at multiple sites. The multiple administrations may be rendered simultaneously or may be administered over a continuous period of time.

Also contemplated in the present invention is the administration of a CD20-specific binding molecule composition in conjunction with a second agent. Second agents contemplated by the invention are listed in the paragraphs below.

A second agent may be a B-cell-associated molecule. Other B-cell-associated molecules contemplated by the invention include binding molecules which bind to B-cell surface molecules that are not CD20. B-cell-associated molecules include, but are not limited to, CD19 (B-lymphocyte antigen CD19, also referred to as B-lymphocyte surface antigen B4, or Leu-12), CD21, CD22 (B-cell receptor CD22, also referred to as Leu-14, B-lymphocyte cell adhesion molecule, or BL-CAM), CD23, CD37, CD40 (B-cell surface antigen CD40, also referred to as Tumor Necrosis Factor receptor superfamily member 5, CD40L receptor, or Bp50), CD80 (T lymphocyte activation antigen CD80, also referred to as Activation B7-1 antigen, B7, B7-1, or BB1), CD86 (T lymphocyte activation antigen CD86, also referred to as Activation B7-2 antigen, B70, FUN-1, or BU63), CD137 (also referred to as Tumor Necrosis Factor receptor superfamily member 9), CD152 (also referred to as cytotoxic T-lymphocyte protein 4 or CTLA-4), L6 (Tumor-associated antigen L6, also referred to as Transmembrane 4 superfamily member 1, Membrane component surface marker 1, or M3S1), CD30 (lymphocyte activation antigen CD30, also referred to as Tumor Necrosis Factor receptor superfamily member 8, CD30L receptor, or Ki-1), CD50 (also referred to as Intercellular adhesion molecule-3 (ICAM3), or ICAM-R), CD54 (also referred to as Intercellular adhesion molecule-1 (ICAM1), or Major group rhinovirus receptor), B7-H1 (ligand for an immunoinhibitory receptor expressed by activated T cells, B-cells, and myeloid cells, also referred to as PD-L1; see Dong, et al., “B7-H1, a third member of the B7 family, co-stimulates T-cell proliferation and interleukin-10 secretion,” Nat. Med. 1999, 5:1365-1369), CD134 (also referred to as Tumor Necrosis Factor receptor superfamily member 4, OX40, OX40L receptor, ACT35 antigen, or TAX-transcriptionally activated glycoprotein 1 receptor), 41BB (4-1BB ligand receptor, T-cell antigen 4-1BB, or T-cell antigen ILA), CD153 (also referred to as Tumor Necrosis Factor ligand superfamily member 8, CD30 ligand, or CD30-L), CD154 (also referred to as Tumor Necrosis Factor ligand superfamily member 5, TNF-related activation protein, TRAP, or T cell antigen Gp39) and Toll receptors. The above list of construct targets and/or target antigens is exemplary only and is not exhaustive.

Examples of chemotherapeutic agents contemplated as second agents include, but are not limited to alkylating agents, such as nitrogen mustards (e.g., mechlorethamine, cyclophosphamide, ifosfamide, melphalan, and chlorambucil); nitrosoureas (e.g., carmustine (BCNU), lomustine (CCNU), and semustine (methyl-CCNU)); ethylenimines and methyl-melamines (e.g., triethylenemelamine (TEM), triethylene thiophosphoramide (thiotepa), and hexamethylmelamine altretamine)); alkyl sulfonates (e.g., buslfan); and triazines (e.g., dacabazine (DTIC)); antimetabolites, such as folic acid analogs (e.g., methotrexate, trimetrexate, and pemetrexed (multi-targeted antifolate)); pyrimidine analogs (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogs (e.g., 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; certain natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; certain radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (MIH) and procarbazine.

Non-limiting examples of chemotherapeutic agents, radiotherapeutic agents and other active and ancillary agents are also shown in Table 1.

TABLE 1

Alkylating agents
Nitrogen mustards
mechlorethamine
cyclophosphamide
ifosfamide
melphalan
chlorambucil
Nitrosoureas
carmustine (BCNU)
lomustine (CCNU)
semustine (methyl-CCNU)
Ethylenimine/Methyl-melamine
thriethylenemelamine (TEM)
triethylene thiophosphoramide
(thiotepa)
hexamethylmelamine
(HMM, altretamine)
Alkyl sulfonates
busulfan
Triazines
dacarbazine (DTIC)
Antimetabolites
Folic Acid analogs
methotrexate
Trimetrexate
Pemetrexed
(Multi-targeted antifolate)
Pyrimidine analogs
5-fluorouracil
fluorodeoxyuridine
gemcitabine
cytosine arabinoside
(AraC, cytarabine)
5-azacytidine
2,2′-difluorodeoxy-cytidine
Purine analogs
6-mercaptopurine
6-thioguanine
azathioprine
2′-deoxycoformycin
(pentostatin)
erythrohydroxynonyl-adenine (EHNA)
fludarabine phosphate
2-chlorodeoxyadenosine
(cladribine, 2-CdA)
Type I Topoisomerase Inhibitors
camptothecin
topotecan
irinotecan
Biological response modifiers
G-CSF
GM-CSF
Differentiation Agents
retinoic acid derivatives
Hormones and antagonists
Adrenocorticosteroids/antagonists
prednisone and equivalents
dexamethasone
ainoglutethimide
Progestins
hydroxyprogesterone caproate
medroxyprogesterone acetate
megestrol acetate
Estrogens
diethylstilbestrol
ethynyl estradiol/equivalents
Antiestrogen
tamoxifen
Androgens
testosterone propionate
fluoxymesterone/equivalents
Antiandrogens
flutamide
gonadotropin-releasing
hormone analogs
leuprolide
Nonsteroidal antiandrogens
Flutamide
Natural products
Antimitotic drugs
Taxanes
paclitaxel
Vinca alkaloids
vinblastine (VLB)
vincristine
vinorelbine
Taxotere ® (docetaxel)
estramustine
estramustine phosphate
Epipodophylotoxins
etoposide
teniposide
Antibiotics
actimomycin D
daunomycin (rubido-mycin)
doxorubicin (adria-mycin)
mitoxantroneidarubicin
bleomycin
splicamycin (mithramycin)
mitomycinC
dactinomycin
aphidicolin
Enzymes
L-asparaginase
L-arginase
Radiosensitizers
metronidazole
misonidazole
desmethylmisonidazole
pimonidazole
etanidazole
nimorazole
RSU 1069
EO9
RB 6145
SR4233
nicotinamide
5-bromodeozyuridine
5-iododeoxyuridine
bromodeoxycytidine
Miscellaneous agents
Platinum coordination complexes
cisplatin
Carboplatin
oxaliplatin
Anthracenedione
mitoxantrone
Substituted urea
hydroxyurea
Methylhydrazine derivatives
N-methylhydrazine (MIH)
procarbazine
Adrenocortical suppressant
mitotane (o,p′-DDD)
ainoglutethimide
Cytokines
interferon (α, β, γ)
interleukin-2
Photosensitizers
hematoporphyrin derivatives
Photofrin ®
benzoporphyrin derivatives
Npe6
tin etioporphyrin (SnET2)
pheoboride-a
bacteriochlorophyll-a
naphthalocyanines
phthalocyanines
zinc phthalocyanines
Radiation
X-ray
ultraviolet light
gamma radiation
visible light
infrared radiation
microwave radiation

Second agents contemplated by the invention for treatment of autoimmune diseases may also include immunosuppressive agents, which act to suppress or mask the immune system of the individual being treated. Immunosuppressive agents include, for example, non-steroidal anti-inflammatory drugs (NSAIDs), analgesiscs, glucocorticoids, disease-modifying antirheumatic drugs (DMARDs) for the treatment of arthritis, or biologic response modifiers. Compositions in the DMARD description are also useful in the treatment of many other autoimmune diseases aside from RA.

Exemplary NSAIDs are chosen from the group consisting of ibuprofen, naproxen, naproxen sodium, Cox-2 inhibitors such as VIOXX® and CELEBREX®, and sialylates. Exemplary analgesics are chosen from the group consisting of acetaminophen, oxycodone, tramadol of proporxyphene hygrochloride. Exemplary glucocorticoids are chosen from the group consisting of cortisone, dexamethosone, hydrocortisone, methylprednisolone, prednisolone, or prednisone. Exemplary biological response modifiers include, but are not limited to, molecules directed against cell surface markers (e.g., CD4, CD5, CTLA4, etc.), abatacept, cytokine inhibitors, such as the TNF antagonists (e.g. etanercept (ENBREL®), adalimumab (HUMIRA®), and infliximab (REMICADE®)), chemokine inhibitors and adhesion molecule inhibitors. The biological response modifiers include monoclonal antibodies as well as recombinant forms of molecules. Exemplary DMARDs include, but are not limited to, azathioprine, cyclophosphamide, cyclosporine, methotrexate, penicillamine, leflunomide, sulfasalazine, hydroxychloroquine, Gold [oral (auranofin) and intramuscular] and minocycline. Thus, for example, the present inventive binding proteins can be used for treatment of RA in combination with DMARDs such as methotrexate (MTX), sulfasalazine (SSZ) or leflunomide (LEF); for treatment of lupus (SLE) with DMARDs, steroids, cyclophosphamide or CELLCEPT®; and for treatment of MS with various disease-modifying agents such as interferons (interferon beta-1a (AVONEX® and REBIF®) or interferon beta-1b (BETASERON® or BETAFERON®)), glatiramer acetate (COPAXONE®), mitoxantrone, or natalizumab (TYSABRI®).

It is contemplated that the CD20-specific binding molecule composition and the second agent may be given simultaneously in the same formulation. Alternatively, the agents are administered in a separate formulation and administered concurrently, with concurrently referring to agents given within 30 minutes of each other.

In another aspect, the second agent is administered prior to administration of the CD20-specific binding molecule composition. Prior administration refers to administration of the second agent within the range of one week prior to treatment with the antibody, up to 30 minutes before administration of the antibody. It is further contemplated that the second agent is administered subsequent to administration of the CD20-specific binding molecule composition. Subsequent administration is meant to describe administration from 30 minutes after antibody treatment up to one week after antibody administration.

It is further contemplated that when the CD20-specific binding molecule is administered in combination with a second agent, wherein the second agent is a cytokine or growth factor, or a chemotherapeutic agent, the administration also includes use of a radiotherapeutic agent or radiation therapy. The need for a radiation therapy, in combination with the administration of a CD20-specific binding molecule composition and the second agent, may be determined by the treating physician.

The amounts of CD20-specific binding molecule composition in a given dosage will vary according to the size of the individual to whom the therapy is being administered as well as the characteristics of the disorder being treated. In exemplary treatments, it may be necessary to administer about 1 mg/day, about 5 mg/day, about 10 mg/day, about 20 mg/day, about 50 mg/day, about 75 mg/day, about 100 mg/day, about 150 mg/day, about 200 mg/day, about 250 mg/day, about 400 mg/day, about 500 mg/day, about 800 mg/day, about 1000 mg/day, about 1600 mg/day or about 2000 mg/day. The doses may also be administered based on weight of the patient, at a dose of 0.01 to 50 mg/kg. In a related embodiment, the CD20-specific binding molecule may be administered in a dose range of 0.015 to 30 mg/kg. In an additional embodiment, the CD20-specific binding molecule is administered in a dose of about 0.015, about 0.05, about 0.15, about 0.5, about 1.5, about 5, about 15 or about 30 mg/kg.

Standard dose-response studies, first in animal models and then in clinical testing, can reveal optimal dosages for particular diseases and patient populations.

In certain embodiments, the administration of the CD20-specific binding molecule composition decreases or reduces the B-cell population by at least about 20% after treatment. In one embodiment, the B-cell population is decreased or reduced by at least about 20, about 30, about 40, about 50, about 60, about 70, about 80, about 90 or about 100%. B-cell depletion is defined as a decrease in absolute B-cell count below the lower limit of the normal range. B-cell recovery is defined as a return of absolute B-cell count to either of the following: 1) 70% of subject's baseline value; or 2) normal range.

In certain embodiments, the administration of the CD20-specific binding molecule composition also results in enhanced apoptosis in particular B-cell subsets. Apoptosis refers to the induction of programmed cell death of a cell, manifested and assessed by DNA fragmentation, cell shrinkage, cell fragmentation, formation of membrane vesicles, or alteration of membrane lipid composition as assessed by annexin V staining.

In certain embodiments, the administration of the CD20-specific binding molecule composition results in desired clinical effects in the disease or disorder being treated. For example, in patients affected by rheumatoid arthritis, administration of a CD20 molecule of the invention improves the patient's condition by a clinically significant amount [e.g., achieves the American College of Rheumatology Preliminary Detection of Improvement (ACR20)], and/or an improvement of 20% in tender and swollen joint and 20% improvement in 3/5 remaining ACR measures (Felson et al., Arthritis Rheum. 1995, 38:727-35). Biological measures for improvement in an RA patient after administration of a CD20-specific binding molecule include measurement of changes in cytokine levels, measured via protein or RNA levels. Cytokines of interest include, but are not limited to, TNF-α, IL-1, interferons, Blys, and APRIL. Cytokine changes may be due to reduced B cell numbers or decreased activated T cells. In RA patients, markers relevant to bone turnover (bone resorption or erosion) are measured before and after administration of CD20-specific binding molecules. Relevant markers include, but are not limited to, alkaline phosphatase, osteocalcin, collagen breakdown fragments, hydroxyproline, tartrate-resistant acid phosphotase, and RANK ligand (RANKL). Other readouts relevant to the improvement of RA include measurement of C reactive protein (CRP) levels, erythrocyte sedimentation rate (ESR), rheumatoid factor, CCP (cyclic citrullinated peptide) antibodies and assessment of systemic B cell levels and lymphocyte count via flow cytometry. Specific factors can also be measured from the synovium of RA patients, including assessment of B cell levels in synovium from synovium biopsy, levels of RANKL and other bone factors and cytokines set out above. Additional biomarkers for RA include CRP and SAA.

In a related aspect, the effects of CD20-specific binding molecule treatment on other diseases is measured according to standards known in the art. For example, it is contemplated that Crohn's disease patients receiving treatment with a CD20-specific binding molecule achieve an improvement in Crohn's Disease Activity Index (CDAI) in the range of about 50 to about 70 units, wherein remission is at 150 units (Simonis et al, Scand. J Gastroent. 1998, 33:283-8). A score of 150 or 200 is considered normal, while a score of 450 is considered a severe disease score. It is further desired that administration of the CD20-specific binding molecule results in a reduction in perinuclear anti-neutrophil antibody (pANCA) and anti-Saccharomyces cervisiae antibody (ASCA) in individuals affected by inflammatory bowel disease.

It is further contemplated that adult and juvenile myositis patients receiving treatment with a CD20-specific binding molecule of the invention achieve an improvement in core set of evaluations, such as 3 out of 6 of the core set measured improved by approximately 20%, with not more than 2 of the core measurements worse by approximately 25% (see Rider et al., Arthritis Rheum. 2004, 50:2281-90).

It is further contemplated that SLE patients receiving treatment with a CD20-specific binding molecule of the invention achieve an improvement in Systemic Lupus Activity Measure (SLAM) or SLE Disease Activity Index (SLEDAI) score of at least 1 point (Gladman et al, J Rheumatol 1994, 21:1468-71) (Tan et al., Arthritis Rheum. 1982, 25:1271-7). A SLAM score of >5, or SLEDAI score >2, is considered clinically active disease. A response to treatment may be defined as improvement or stabilization over the in 2 disease activity measures (the SLE Disease Activity Index [SLEDAI] and the Systemic Lupus Activity Measure) and 2 quality of life measures (patient's global assessment and the Krupp Fatigue Severity Scale) (Petri et al., Arthritis Rheum. 2004, 50:2858-68.) It is further desired that administration of the CD20-specific binding molecule to SLE patients results in a reduction in anti-double-stranded DNA antibodies. Alternatively, improvement may be gauged using the British Isles Lupus Assessment Group Criteria (BILAG). Additional biomarkers for SLE include B cell subsets (naive, memory, transitional); CD40L; complement, anti ds-DNA, C1Q; urinary biomarkers (TWEAK, MIF). In addition, with respect to differentiation markers, FcgammaRIII status (high or low affinity) can be used to correlate B cell depletion and efficacy. Reduced complement activation may also confer a safety advantage (based on C3a, C4a, Bb binding).

It is further contemplated that multiple sclerosis patients receiving treatment with a CD20-specific binding molecule of the invention achieve an improvement in clinical score on the Kurtzke Expanded Disability status scale (EDSS) (Kurtzke, F., Neurology 1983, 33:1444-52) of at least 0.5, or a delay in worsening of clinical disease of at least 1.0 on the Kurtzke scale (Rudick et al., Neurology 1997, 49:358-63).

It is further contemplated that patients suffering from IIM receiving treatment of a CD20-specific binding molecule of the invention achieve a reduction in at least one of five criteria set out in the Idiopathic Inflammatory Myopathy Criteria (IIMC) assessment (Miller, F., supra). It is further contemplated that administration of a CD20-specific binding molecule of the invention to IIM patients results in a reduction in IIM-associated factors selected from the group consisting of creatine kinase (CK), lactate dehydrogenase, aldolase, C-reactive protein, aspartate aminotransferase (AST), alanine aminotransferase (ALT), and antinuclear autoantibody (ANA), myositis-specific antibodies (MSA), and antibody to extractable nuclear antigens. Alternatively, patients meeting 3 out of 6 of the criteria set out in Rider et al., Arthritis Rheum. 2004, 50:2281-90, may be the subject of treatment according to the invention, with worsening in no more than 2 criteria.

In a still further embodiment, patients suffering from a B cell cancer receive treatment with a CD20-specific binding molecule of the invention and demonstrate an overall beneficial response to the CD20-specific binding molecule, based on clinical criteria well-known and commonly used in the art, and as described below, such as a decrease in tumor size, decrease in tumor number and/or an improvement in disease symptoms.

For example, the U.S. National Cancer Institute (NCI) has divided some of the classes of cancers into the clinical categories of “indolent” and “aggressive” lymphomas. Indolent lymphomas include follicular cell lymphomas, separated into cytology “grades,” diffuse small lymphocytic lymphoma/chronic lymphocytic leukemia (CLL), lymphoplasmacytoid/Waldenstrom's Macroglobulinemia, Marginal zone lymphoma and Hairy cell leukemia. Aggressive lymphomas include diffuse mixed and large cell lymphoma, Burkitt's lymphoma/diffuse small non-cleaved cell lymphoma, Lymphoblastic lymphoma, Mantle cell lymphoma and AIDS-related lymphoma. In some cases, the International Prognostic Index (IPI) is used in cases of aggressive and follicular lymphoma. Factors to consider in the IPI include Age (<60 years of age versus >60 years of age), serum lactate dehydrogenase (levels normal versus elevated), performance status (0 or 1 versus 2-4) (see definition below), disease stage (I or II versus III or IV), and extranodal site involvement (0 or 1 versus 2-4). Patients with 2 or more risk factors have less than a 50% chance of relapse-free and overall survival at 5 years.

Performance status in the aggressive IPI is defined as follows: Grade Description: 0 Fully active, able to carry on all pre-disease performance without restriction; 1 Restricted in physically strenuous activity but ambulatory and able to carry out work of a light or sedentary nature, e.g., light house work, office work; 2 Ambulatory and capable of all selfcare but unable to carry out any work activities, up to and about more than 50% of waking hours; 3 Capable of only limited selfcare, confined to bed or chair more than 50% of waking hours; 4 Completely disabled, unable to carry on any selfcare, totally confined to bed or chair; and, 5 Dead. (See., The International Non-Hodgkin's Lymphoma Prognostic Factors Project. A predictive model for aggressive non-Hodgkin's lymphoma. N Engl J Med. 329:987-94, 1993)

Typically, the grade of lymphoma is clinically assessed using the criterion that low-grade lymphoma usually presents as a nodal disease and is often indolent or slow-growing. Intermediate- and high-grade disease usually presents as a much more aggressive disease with large extranodal bulky tumors.

The Ann Arbor classification system is also used to measure progression of tumors, especially non-Hodgkins lymphomas. In this system, stages I, II, III, and IV of adult NHL can be classified into A and B categories depending on whether the patient has well-defined generalized symptoms (B) or not (A). The B designation is given to patients with the following symptoms: unexplained loss of more than 10% body weight in the 6 months prior to diagnosis, unexplained fever with temperatures above 38° C. and drenching night sweats. Definitions of the stages are as follows: Stage I-involvement of a single lymph node region or localized involvement of a single extralymphatic organ or site. Stage III-involvement of two or more lymph node regions on the same side of the diaphragm or localized involvement of a single associated extralymphatic organ or site and its regional lymph nodes with or without other lymph node regions on the same side of the diaphragm. Stage III-involvement of lymph node regions on both sides of the diaphragm, possibly accompanying localized involvement of an extralymphatic organ or site, involvement of the spleen, or both. Stage IV-disseminated (multifocal) involvement of one or more extralymphatic sites with or without associated lymph node involvement or isolated extralymphatic organ involvement with distant (non-regional) nodal involvement. For further details, see The International Non-Hodgkin's Lymphoma Prognostic Factors Project: A predictive model for aggressive non-Hodgkin's lymphoma, New England J. Med. (1993) 329:987-994.

In one aspect, a therapeutic effect of the CD20-specific binding molecule is determined by the level of response, for example a partial response is defined as tumor reduction to less than one-half of its original size. A complete response is defined as total elimination of disease confirmed by clinical or radiological evaluation. In one embodiment, individuals receiving treatment with a CD20-specific binding molecule of the invention demonstrate at least a partial response to the treatment.

According to the Cheson criteria for assessing NHL developed in collaboration with the National Cancer Institute (Cheson et al., J Clin Oncol. 1999, 17:1244; Grillo-Lopez et al., Ann Oncol. 2000, 11:399-408), a complete response is obtained when there is a complete disappearance of all detectable clinical and radiographic evidence of disease and disease-related symptoms, all lymph nodes have returned to normal size, the spleen has regressed in size, and the bone marrow is cleared of lymphoma.

An unconfirmed complete response is obtained when a patient shows complete disappearance of the disease and the spleen regresses in size, but lymph nodes have regressed by more than 75% and the bone marrow is indeterminate. An unconfirmed complete response meets and exceeds the criteria for partial response. An overall response is defined as a reduction of at least 50 percent in overall tumor burden.

Similar criteria have been developed for various other forms of cancers or hyperproliferative diseases and are readily available to a person of skill in the art. See, e.g., Cheson et al., Clin Adv Hematol Oncol. 2006, 4:4-5, which describes criteria for assessing CLL; Cheson et al., J Clin Oncol. 2003, 21:4642-9, which describes criteria for AML; Cheson et al., Blood 2000, 96:3671-4, which describes criteria for myelodysplastic syndromes.

In another aspect, a therapeutic response to a CD20-binding molecule in patients having a B cell cancer is manifest as a slowing of disease progression compared to patients not receiving therapy. Measurement of slowed disease progression or any of the above factors may be carried out using techniques well-known in the art, including bone scan, CT scan, gallium scan, lymphangiogram, MRI, PET scans, ultrasound, and the like.

In a related aspect, to determine the efficacy of CD20-binding molecule treatment, the number of B cells in a biological sample of the individual is measured. In one embodiment, the biological sample is selected from blood, tumor biopsy, lymph nodes, tonsils, bone marrow, thymus and other lymphocyte-rich tissue. Lymphocyte-rich tissue is tissue particularly rich in lymphocyte cells, including but not limited to, lymph nodes and related organs (spleen, bone marrow, tonsils, thymus, mucosal lymph tissue), tumors and areas of inflammation.

It will also be apparent that dosing may be modified if traditional therapeutics are administered in combination with therapeutics of the invention.

It is further contemplated that an individual being treated by a method of the invention may be re-treated, for example, if symptoms of disease reappear or the pharmacokinetics and/or pharmodynamics of the therapeutic make such re-treatment advisable. In one embodiment, the individual treated with a CD20-specific binding molecule of the invention is administered another CD20-specific binding molecule. Based upon ordinary skill in the art, a clinician would be able to identify when re-treatment is indicated based upon, for example, reappearance of disease symptoms or recovery of the individual's B cells to a level requiring re-treatment. Examples of other measurements or markers of clinical criteria and outcome are described further herein. An individual treated by a method of the invention may be placed on a maintenance schedule of treatment, wherein the individual is re-treated with the CD20-specific binding molecule based on pharmacokinetic/pharmacodynamic properties of the CD20-specific binding molecule. Such a maintenance treatment is typically administered anywhere from about three months to about two years after the initial treatment. Exemplary pharmacodynamic data include, but are not limited to, biological measures for improvement of disease as described herein, such as levels of the CD20-specific binding molecule in serum, improvement in disease assessment (e.g., by ACR, SLAM or IPI), change in cytokine or surface marker expression, levels of autoantibodies, and change in tumor size. It is further understood in the art that differences in individual responses to treatment by methods of the invention may necessitate differences in timing of re-treatment with the CD20-specific binding molecule.

As an additional aspect, the invention includes kits which comprise one or more compounds or compositions packaged in a manner which facilitates their use to practice methods of the invention. In one embodiment, such a kit includes a CD20-specific binding molecule compound or composition described herein (e.g., a composition comprising a CD20-specific binding molecule alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the antibody composition.

The present invention also comprises articles of manufacture. Such articles comprise at least one CD20-specific binding molecule, optionally together with a pharmaceutical carrier or diluent, and at least one label describing a method of use of the CD20-specific binding molecule according to the invention. Such articles of manufacture may also optionally comprise at least one second agent for administration in connection with the CD20-specific binding molecule.

### EXAMPLES

### Example 1

### Binding of Anti-CD20 SMIPs to Primary B cells

Primary Human B Cells

To determine the binding of anti-CD20 SMIPs to primary B cells, we isolated primary B cells from buffy coats using negative selection B cell isolation kit (StemCell Technologies). We incubated the harvested cells with varying concentrations of anti-CD20

SMIP for 30 minutes on ice. Cells were then washed in 0.5% BSA/PBS, and stained with anti-human IgG-PE for 30 minutes and analyzed by flow cytometry (MFI) on FacsCalibur.

As demonstrated in FIG. 1, all anti-CD20 SMIPs analyzed in this example (TRU-015, 018008 csc, 018008sccp, 2LM 19-3 csc, 2LM 19-3 sccp, 2LM 20-4 csc, 2LM 16 csc, 2LM 16 scc, 2LM 16 sccp, 2LM 20-4 sccp, 009csc, 009 scc, 009 sccp, 018011 csc, 018011 scc, 018011 sccp) had comparable binding affinities to CD20 on human B cells.

TABLE 2
Binding on Primary B cells with P/S mutation SMIPS

| | MFI | | MFI | | MFI | | MFI |
|---|---|---|---|---|---|---|---|
| 10 ug/ml | | 1.1 ug/ml | | 0.37 ug/ml | | 0.12 ug/ml | |
| Rituxan | 198.7 | Rituxan | 172.9 | Rituxan | 137.3 | Rituxan | 105.7 |
| TRU-015 | 149.3 | TRU-015 | 88.0 | TRU-015 | 64.8 | TRU-015 | 42.8 |
| 2Lm 20-4 sccp | 80.1 | 2Lm 20-4 sccp | 66.0 | 2Lm 20-4 sccp | 43.2 | 2Lm 20-4 sccp | 25.6 |
| 18008 sccp | 79.5 | 2LM 20-4 sccp p/s | 48.8 | 2LM 20-4 sccp p/s | 33.4 | 2Lm 19-3 sccp | 17.7 |
| 2Lm 19-3 sccp | 61.4 | 2Lm 19-3 sccp | 48.7 | 2Lm 19-3 sccp | 31.0 | 2LM 20-4 sccp p/s | 17.6 |
| 2Lm 16 sccp p/s | 54.3 | 18008 sccp | 44.8 | 009 sccp | 28.4 | 009 sccp | 17.0 |
| 2Lm 16 csc p/s | 52.2 | 2Lm 16 sccp p/s | 42.8 | 2Lm 16 sccp p/s | 25.0 | 2Lm 16 sccp p/s | 13.5 |
| 009 sccp | 51.9 | 2Lm 19-3 sccp p/s | 41.8 | 18008 sccp | 24.8 | 18008 sccp | 13.0 |
| 2LM 20-4 sccp p/s | 50.6 | 009 sccp | 39.8 | 2Lm 19-3 sccp p/s | 22.4 | 2Lm 19-3 sccp p/s | 12.7 |
| 2Lm 19-3 sccp p/s | 43.5 | 009 sccp p/s | 32.7 | 009 sccp p/s | 20.2 | 009 sccp p/s | 10.4 |
| 009 csc p/s | 42.2 | 009 csc p/s | 31.6 | 009 csc p/s | 19.0 | 009 csc p/s | 9.8 |
| 009 sccp p/s | 40.4 | 2Lm 16 csc p/s | 30.5 | 2Lm 16 csc p/s | 15.2 | 2Lm 16 csc p/s | 9.3 |
| 10 ug/ml | | 1.1 ug/ml | | 0.12 ug/ml | | 0.04 ug/ml | |
| Rituxan | 171.6 | Rituxan | 93.0 | Rituxan | 82.3 | Rituxan | 43.1 |
| TRU-015 | 123.6 | TRU-015 | 53.4 | 2LM 20-4 sccp p/s | 38.3 | TRU-015 | 19.9 |
| 2Lm 20-4 sccp | 67.8 | 2LM 20-4 sccp p/s | 39.2 | TRU-015 | 31.4 | 2LM 20-4 sccp p/s | 16.2 |
| 2LM 20-4 sccp p/s | 66.8 | 2Lm 19-3 sccp p/s | 35.9 | 2Lm 19-3 sccp p/s | 31.0 | 2Lm 20-4 sccp | 14.4 |
| 18008 sccp | 64.7 | 2Lm 19-3 sccp | 35.2 | 2Lm 20-4 sccp | 27.8 | 2Lm 16 sccp p/s | 14.0 |
| 2Lm 19-3 sccp | 60.7 | 2Lm 16 sccp p/s | 34.9 | 2Lm 16 sccp p/s | 26.4 | 2Lm 19-3 sccp | 13.4 |
| 2Lm 16 csc p/s | 59.6 | 2Lm 20-4 sccp | 34.1 | 2Lm 19-3 sccp | 23.4 | 2Lm 19-3 sccp p/s | 13.0 |
| 009 csc p/s | 56.8 | 009 csc p/s | 31.0 | 009 csc p/s | 21.5 | 009 csc p/s | 11.7 |
| 2Lm 19-3 sccp p/s | 56.1 | 009 sccp p/s | 28.1 | 009 sccp p/s | 21.4 | 009 sccp p/s | 10.2 |
| 009 sccp | 50.7 | 2Lm 16 csc p/s | 26.1 | 009 sccp | 16.8 | 2Lm 16 csc p/s | 8.8 |
| 2Lm 16 sccp p/s | 48.2 | 009 sccp | 25.1 | 2Lm 16 csc p/s | 16.0 | 009 sccp | 8.5 |
| 009 sccp p/s | 46.9 | 18008 sccp | 20.7 | 18008 sccp | 12.9 | 18008 sccp | 6.1 |

Malignant B Lymphoid Cells

Binding of anti-CD20 018011, TRU-015 and RITUXAN® was examined using a panel of 5 human B-lymphoma cell lines (BCL): NU-DHL1, Ramos, SU-DHL4, SU-DHL5, and WSU-DLCL2. Each of these cell lines was derived from a distinct non-Hodgkin's B-cell lymphoma patient. Briefly, increasing concentrations of CD20-binders were incubated with 100,000 BCL for 30 min at 4° C. Cells were then washed twice with PBS containing 1% BSA to remove unbound antibody and then a FITC labeled goat anti-human (H+ L) secondary antibody (100 fold diluted) was added for 30 min at 4° C. The cells were again washed twice to remove unbound secondary antibody and then resuspended in 1% formaldehyde (in PBS) with 1% BSA. Fluorescence intensity of each sample was measured using a Becton Dickinson FACSORT flow cytometer. Results are expressed as the geometric mean (GeoMean) of the fluorescent intensity.

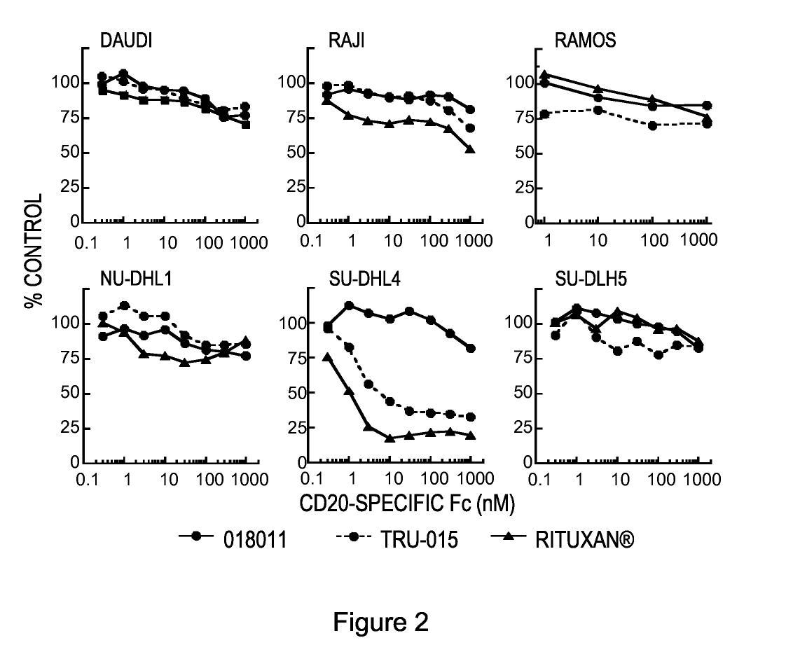

FIG. 2 shows the results in the five cell lines. 018011 demonstrated dose-dependent binding to each of these cell lines. 018008 and 2Lm20-4 also bound to the five cell lines (data not shown). Binding of 018011 to Ramos and SWU-DLCL2 cells was confirmed by immunofluorescence (IFA) according to the protocol in Example 5.

### Example 2

### Complement Dependent Cytotoxicity Assay of Anti-CD20 SMIPs

Ramos Cells

To determine the level of complement dependent cytotoxicity (CDC) of the human anti-CD20 SMIPs, we incubated Ramos cells with anti-CD20 SMIPs in the presence of 10% human sera (Quidel) for 3.5 hours at 37° C. We assessed cell death by measuring LDH release from cells (Promega kit).

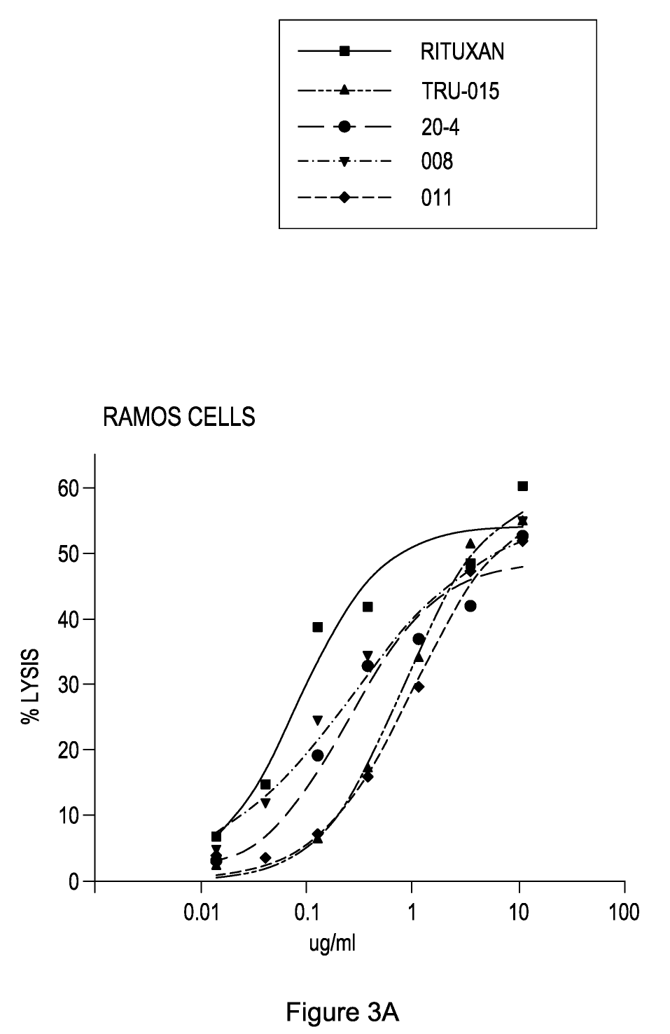

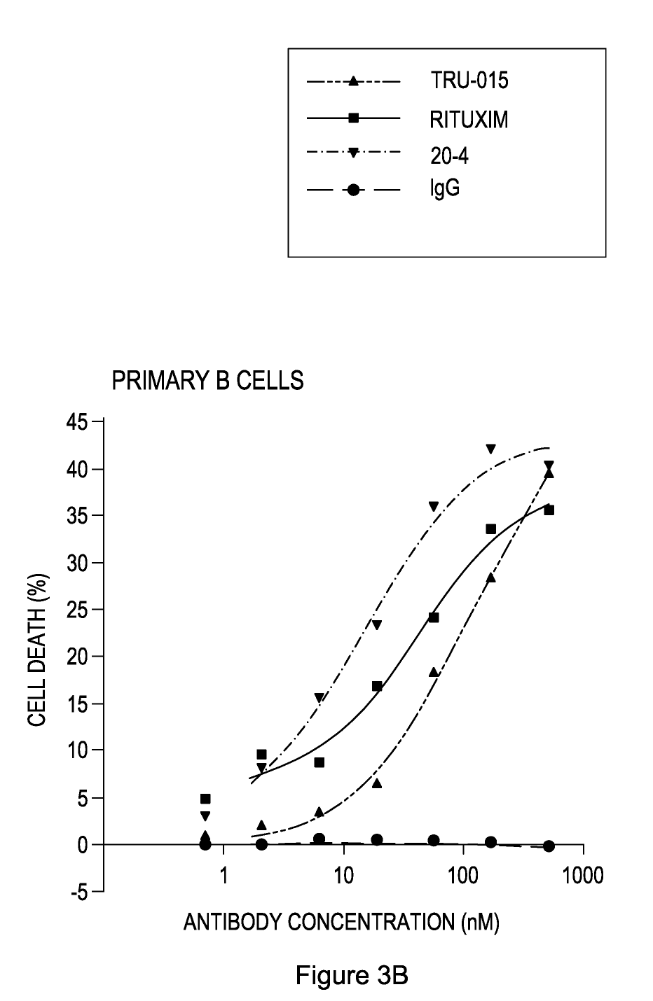

As shown in FIG. 3A, RITUXAN®, TRU-015, 2LM 20-4, 018008, and 018011 had comparable CDC activity against human Ramos B-cells.

TABLE 3
CDC on Ramos cells with P/S mutation SMIPS

| % lysis | | % lysis | | % lysis | | % lysis | |
|---|---|---|---|---|---|---|---|
| 10 ug/ml | | 1.1 ug/ml | | 0.37 ug/ml | | 0.12 ug/ml | |
| 2Lm 16 sccp p/s | 85.7 | 2Lm 19-3 sccp | 58.1 | Rituxan | 60.2 | Rituxan | 56.5 |
| 009 sccp p/s | 83.0 | 18008 sccp | 57.1 | 18008 sccp | 47.9 | 18008 sccp | 32.2 |
| Rituxan | 82.5 | Rituxan | 57.1 | 2Lm 20-4 sccp | 45.4 | 2Lm 19-3 sccp | 30.5 |
| 2Lm 19-3 sccp p/s | 80.3 | TRU-015 | 56.1 | 2Lm 19-3 sccp | 40.8 | 2Lm 20-4 sccp | 20.0 |
| 18008 sccp | 72.5 | 2Lm 20-4 sccp | 52.9 | TRU-015 | 38.8 | TRU-015 | 16.3 |
| TRU-015 | 67.7 | 009 sccp | 50.3 | 009 sccp | 26.1 | 009 sccp p/s | 11.4 |
| 2Lm 20-4 sccp p/s | 66.3 | 2Lm 20-4 sccp p/s | 28.3 | 009 csc p/s | 18.8 | 009 sccp | 11.2 |
| 2Lm 20-4 sccp | 63.2 | 009 sccp p/s | 23.4 | 009 sccp p/s | 12.7 | 2Lm 16 sccp p/s | 9.5 |
| 2Lm 19-3 sccp | 60.1 | 2Lm 19-3 sccp p/s | 21.3 | 2Lm 19-3 sccp p/s | 12.4 | 009 csc p/s | 9.3 |
| 2Lm 16 csc p/s | 56.6 | 2Lm 16 sccp p/s | 18.3 | 2Lm 20-4 sccp p/s | 12.3 | 2Lm 20-4 sccp p/s | 8.9 |
| 009 sccp | 56.4 | 2Lm 16 csc p/s | 16.5 | 2Lm 16 sccp p/s | 11.5 | 2Lm 16 csc p/s | 8.9 |
| 009 csc p/s | 40.9 | 009 csc p/s | 16.2 | 2Lm 16 csc p/s | 9.2 | 2Lm 19-3 sccp p/s | 8.8 |
| 100 nm | | 11 nm | | 4 nm | | 1 nm | |
| 2Lm 16 sccp p/s | 107.1 | 18008 sccp | 66.7 | Rituxan | 65.1 | Rituxan | 67.6 |
| 009 sccp p/s | 98.5 | Rituxan | 66.3 | 18008 sccp | 61.4 | 18008 sccp | 50.0 |
| 2Lm 19-3 sccp p/s | 92.7 | 2Lm 20-4 sccp | 65.5 | 2Lm 20-4 sccp | 56.9 | 2Lm 20-4 sccp | 36.1 |
| 009 sccp | 88.3 | 2Lm 19-3 sccp | 61.3 | 2Lm 19-3 sccp | 51.7 | 2Lm 19-3 sccp | 29.7 |
| 2Lm 20-4 sccp p/s | 87.2 | 009 sccp | 55.7 | 009 sccp | 38.0 | 009 sccp p/s | 27.0 |
| TRU-015 | 86.1 | TRU-015 | 49.7 | TRU-015 | 26.2 | 2Lm 16 sccp p/s | 22.9 |
| 2Lm 20-4 sccp | 82.0 | 2Lm 20-4 sccp p/s | 27.1 | 009 sccp p/s | 21.3 | 2Lm 16 csc p/s | 22.5 |
| Rituxan | 81.9 | 2Lm 16 sccp p/s | 24.7 | 2Lm 16 sccp p/s | 19.6 | 009 sccp | 20.9 |
| 2Lm 19-3 sccp | 79.7 | 009 sccp p/s | 23.8 | 2Lm 19-3 sccp p/s | 18.5 | 2Lm 19-3 sccp p/s | 20.5 |
| 18008 sccp | 73.9 | 2Lm 19-3 sccp p/s | 22.2 | 2Lm 20-4 sccp p/s | 17.4 | TRU-015 | 19.8 |
| 2Lm 16 csc p/s | 69.2 | 2Lm 16 csc p/s | 17.0 | 009 csc p/s | 16.4 | 009 csc p/s | 18.7 |
| 009 csc p/s | 64.3 | 009 csc p/s | 16.0 | 2Lm 16 csc p/s | 14.3 | 2Lm 20-4 sccp p/s | 15.1 |

Primary B Cells

We also isolated primary B cells from buffy coats using negative selection B cell isolation kit (StemCell Technologies). We pre-incubated 5×10⁵ B cells with anti-hCD55 antibody (2 ug/ml) for 10 min at 37° C. We then added anti-CD20 SMIPs and serum (10%: Quidel). After 3.5 hours incubation, we assessed cell death by 7-AAD staining and FACs analysis.

As shown in FIG. 3B, TRU-015, RITUXAN®, and 2LM 20-4 had comparable CDC activity against primary B-cells. No CDC activity against primary B cells were detected when the IgG control was added.

SU-DHL4 B Cells and BJAB Cells

We also investigated the ability of humanized anti-CD20 SMIPs to mediate CDC against SU-DHL4 B-cell lymphoma cells. using fresh human serum as a source of complement. Human whole blood was collected and allowed to clot for 60 min at room temperature after which the tubes were centrifuged to collect serum for CDC analysis. CD20-specific TRU-015 and RITUXAN® were used as positive controls and HER2-specific trastuzumab was used as an isotype-matched nonbinding control in this study.

SU-DHL4B cells were plated in 96 well plates with varying amounts of CD20-binders. Diluted human complement (1:100), prepared from the blood of healthy volunteers, was added to each well. Tests were conducted in triplicate in a final volume of 100 μl/well with medium alone, cells alone, CD20-binders alone and complement alone, all used as controls. After 4 h incubation at 37° C., plates were removed from the incubator and equilibrated to 22° C. (approximately 20-30 minutes).

LDH Release Assay

The CYTOTOX-ONE™ fluorometric method estimates the number of non-viable cells in a cytotoxicity assay. It allows for the rapid fluorescence measurement of the release of lactate dehydrogenase (LDH) from cells with damaged cell membranes. LDH released into the culture medium is measured with a 10-minute coupled enzymatic assay that results in the conversion of Resazurin into Resorufin. The generation of the fluorescent Resorufin product is proportional to the amount of LDH.

Briefly, an equal volume of CYTOTOX-ONE™ was added to each well, shaken gently for 30 seconds, and incubated further at 22° C. for 10 minutes. As a positive control, 2 μl of lysis buffer per well (in triplicates) was added to generate a maximum LDH release from cells. After 10 minutes of incubation, the enzymatic reaction was stopped by adding 50 μl of stop solution and the plates shaken gently for 10 seconds. Fluorescence was measured with a fluorimeter at an excitation wavelength of 560 nm and an emission wavelength of 590 nm.

The percent cytotoxicity was calculated by the following equation:

$$\% Lysis = 100 \times \frac{( Experimental release - Background release )}{( Maximum release - Background release )}$$

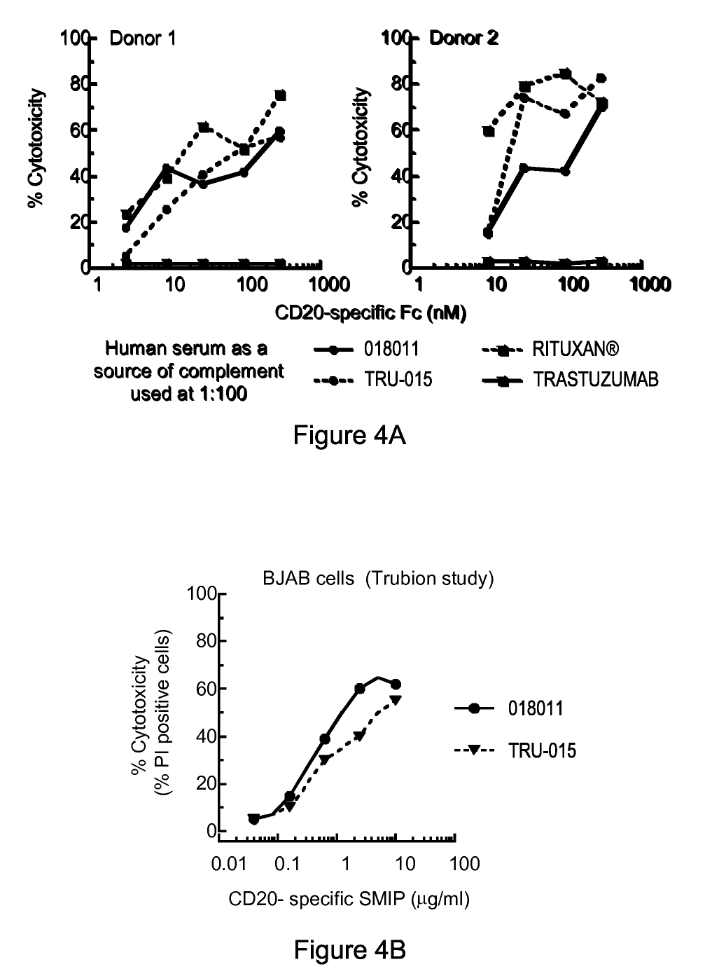

As shown in FIG. 4, 018011 was capable of mediating CDC with human complement from two separate donors and SU-DHL4 B cells. This CDC effect was proportional to concentrations of 018011, TRU-015 and RITUXAN®. Complement from one of the two donors was less supportive of the CDC activity of 018011, which appeared lower than that of RITUXAN®. The significance of this observation with 018011 is unclear.

In a similar study using BJAB cells as the target cell population, 018011 and TRU-015 both produced equivalent concentration-dependent complement-mediated cytotoxicity against BJAB cells (FIG. 4).

### Example 3

### Antibody Dependent Cytotoxicity (ADCC) Assays of Anti-CD20 SMIPs

We determined the level of antibody dependent cytotoxicity (ADCC) (also referred to as FcCC) of the anti-CD20 SMIPs using a number of different target cells.

BJAB Lymphoma Cells

In one experiment, we labeled BJAB lymphoma cells with 0.5 uM CFSE. Labeled cells were then incubated with anti-CD20 binders for 15 minutes, followed by the addition of activated PBMC (which were previously stimulated with IL-2 and IL-12 overnight). After 6 hours of incubation, we stained CFSE target cells (CFSE⁺) with PI and assessed cell death using flow cytometry.

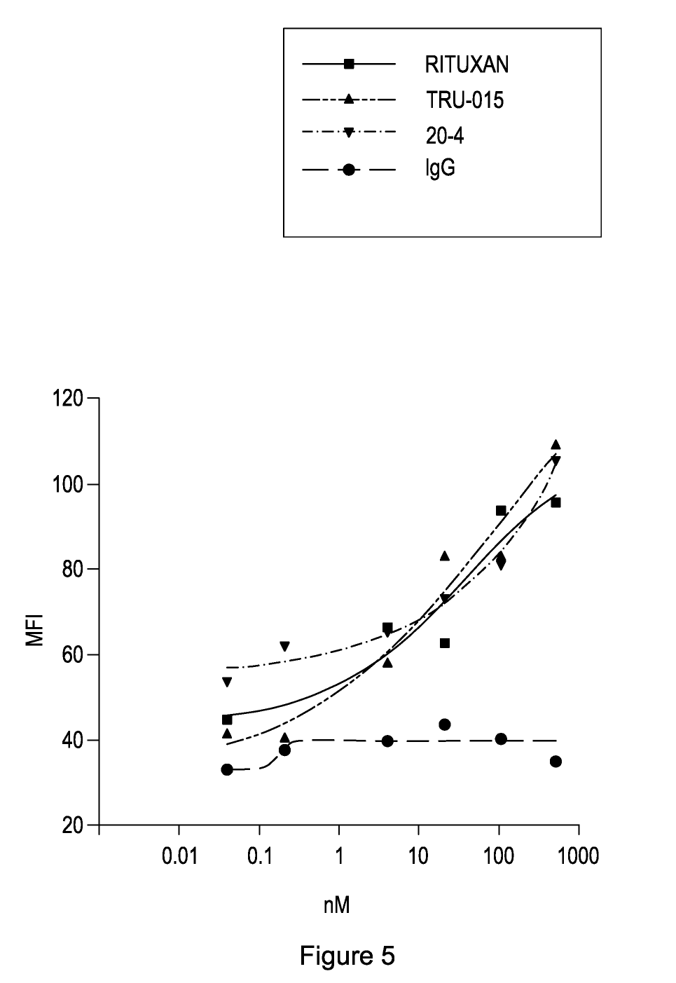

As demonstrated in FIG. 5, RITUXAN®, TRU-015 and 2LM20-4 mediated comparable ADCC activity. Additional experiments using ⁵¹Cr labeled BJAB cells demonstrated ADCC activity of 018008 and 018011 (data not shown).

Ramos B-Cells and SU-DHL4

### Preparation of Effector Cells

PBMNC were isolated by density-gradient centrifugation using (Lymphoprep™ Axis-Shield PoC AS, Norway). Whole blood was collected in a tube containing anticoagulant (heparin). The blood was diluted by addition of an equal volume of 0.9% NaCl and then 6 ml of diluted blood was layered over 3 ml of Lymphoprep solution in a 15 ml conical tube and centrifuged at 800×g for 20 minutes at room temperature. The mononucleocytes recovered from the interface were washed and used in the ADCC and FcCC assays.

ADCC Protocol

Effector and target cells were plated at a ratio of 50:1 in 96 well plates with varying concentrations of CD20-binder added to appropriate wells. Tests were conducted in triplicate at a final volume of 100 μl/well with medium alone, effector cells alone, target cells alone and CD20-binder alone as controls. Fluorescent signal was measured as described above in the CDC assay.

### Results

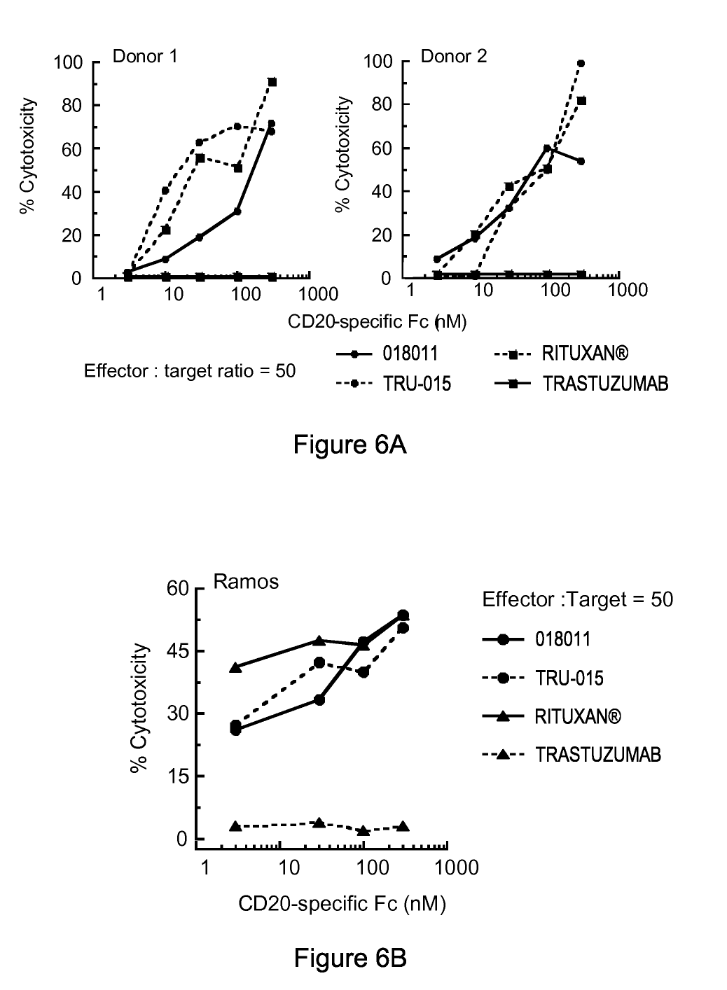

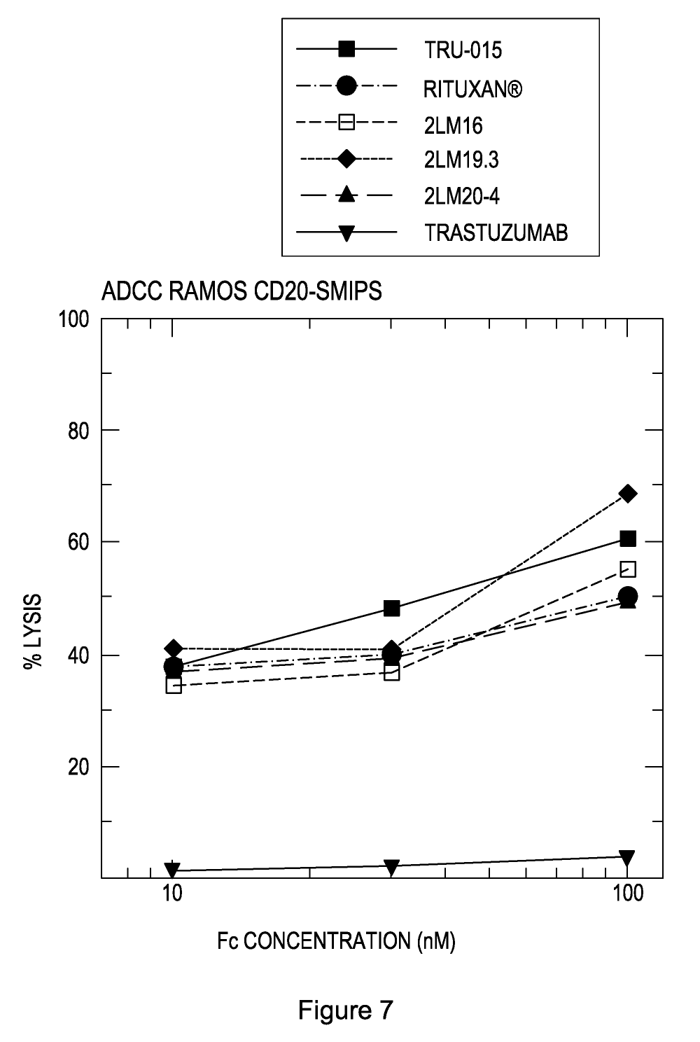

The ability of 018011 to facilitate Fc-mediated cellular cytotoxicity (FcCC) was assessed against CD20+ SU-DHL4 and Ramos BCL. In addition, TRU-015, RITUXAN® and anti-HER2 trastuzumab (used as an isotype-matched nonbinding control Fc) were used in this evaluation. Freshly isolated PBMNC from normal healthy donors were used as a source of effector cells in this assessment. PBMNC include FcγR3/CD16+ NK cells capable of mediating FcCC. In addition to NK cells, monocytes in the PBMNC also express FcγR and have the capability to bring about FcCC. FIG. 6 present the results from experiments using CD20+ SU-DHL4 and Ramos B-lymphoma cells. Each of the CD20 binding agents was able to mediate FcCC in a dose-dependent manner using human effector cells.

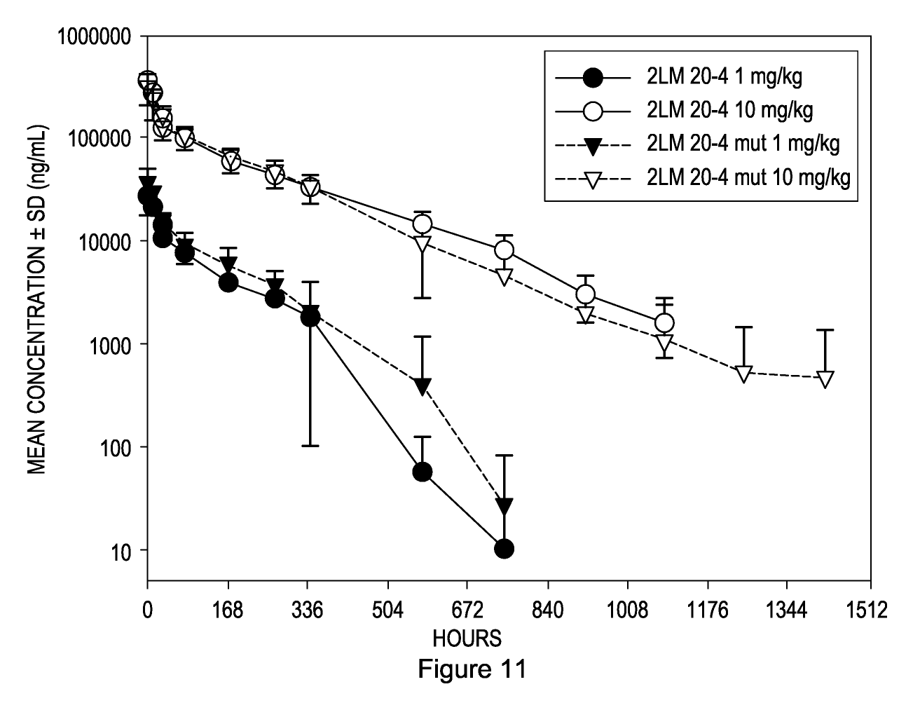

Similar experiments with additional humanized CD20 binding molecules showed ADCC activity on Ramos B cells [FIG. 11].

TABLE 4
Rank of humanized anti-CD20 SMIPs

| Ramos ADCC | Farage ADCC |
|---|---|
| TRU-015 | 2 LM 16 SCCP |
| 2LM 19-3 SCCP | 018008 SCCP |
| 018008 SCCP | TRU-015 |
| 2LM 19-3 CSCS | 2 LM 19-3 CSCS |
| 2 LM 16 SCCS | 2 LM 19-3 SCCP |
| 2 LM 20-4 CSCS | 2 LM 20-4 CSCS |
| 2 LM 16 CSCS | 2 LM 16 CSCS |
| RITUXIMAB | 2 LM 16 SCCS |
| 018009 SCCS | RITUXIMAB |
| 018009 CSCS | 018011 CSCS |
| 018009 SCCP | 2 LM 20-4 SCCP |
| 018011 SCCS | 018011 SCCS |
| 018011 CSCS | 018011 SCCP |
| 2 LM 16 SCCP | 018009 CSCS |
| 018011 SCCP | 018009 SCCS |
| 2 LM 20-4 SCCP | 018009 SCCP |
| 018008 CSCS | 018008 CSCS |

### Example 4

### Pharmacokinetic and Pharmacodynamic Study of Anti-CD20 SMIPs

To investigate the pharmacokinetics and pharmacodynamics of anti-CD20 SMIPs, we conducted a 26-week IV bolus pharmacokinetic study in cynomolgus monkeys. We treated adult female cynomolgus monkeys according to the following:

TABLE 5

| Treatment | | |
|---|---|---|
| Group | Test article | Dose Level (mg/kg) |
| 1 | Rituxan | 1 |
| 2 | | 10 (animals undergoing |
| | | BM aspirates, LN biopsies) |
| 3 | | 10 |
| 4 | 2LM 20-4 | 1 |
| 5 | | 10 (animals undergoing |
| | | BM aspirates, LN biopsies) |
| 6 | | 10 |
| 7 | 2LM 20-4 | 1 |
| 8 | mut Fc | 10 (animals undergoing |
| | (attenuated | BM aspirates, LN biopsies) |
| 9 | CDC) | 10 |

We obtained blood samples, lymph mode biopsies, and bone marrow aspirates from the animal groups to study the test article pharmacokinetics. Whole blood and serum samples were subject to hematology, flow cytometry, and PK analysis; lymph node biopsies were subject to flow cytometry and immunohistochemistry analysis; bone marrow aspirates was analyzed by flow cytometry.

Clinical observations of the animals were normal, none of the test articles was associated with adverse signs. In addition, we noted that hemoglobin levels, and platelet, monocyte, eosinophil and basophil counts were not affected by the test articles.

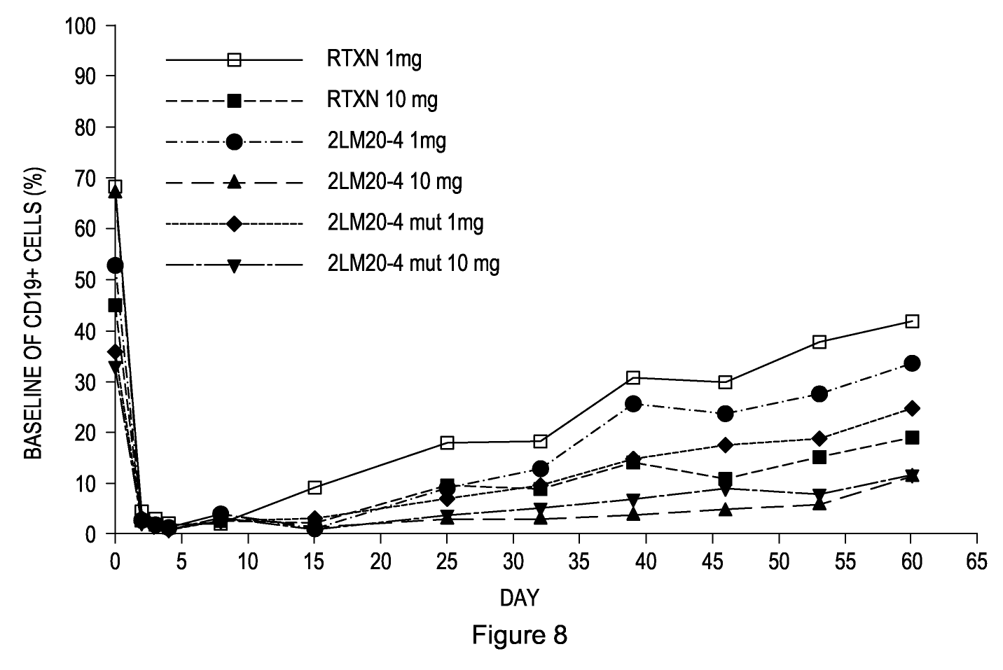

Flow cytometry analysis of peripheral blood showed that those groups treated with 10 mg/kg of 2LM 20-4 (wild type) or 2LM 20-4 mut Fc showed strong and long lasting elimination of CD19⁺ B cells (FIG. 8). Groups treated with 2LM 20-4, 2LM 20-4 mut Fc, and Rituxan showed comparable depletion of peripheral CD19⁺ B cells.

Flow cytometry analysis of bone marrow revealed that groups treated with 2LM 20-4, 2LM 20-4 mut Fc, and Rituxan demonstrated comparable depletion of bone marrow CD19+ B cells on Day 8 (FIG. 9). On day 22, however, the Rituxan-treated group had significantly higher number of CD19+ B cells, as compared to groups treated with 2LM 20-4 and 2LM 20-4 mut Fc (FIG. 9).

Flow cytometry analysis of lymph nodes revealed that 2LM 20-4 demonstrated better efficacy compared to 2LM 20-4 mut Fc, both on Day 8 and Day 22 (FIG. 10). 2LM 20-4 significantly reduced the relative percentage of lymph node CD19⁺ B cells.

We also obtained pharmacokinetic (PK) data of 2LM 20-4 and 2LM 20-4 mut Fc in cynomolgus monkeys after IV administration. Both 2LM 20-4 and 2LM 20-4 showed slow elimination rate when adminstered at 10 mg/kg, with a half-life of 7.2±0.6 days for 2LM 20-4, and 5.5±2.4 days for 2LM 20-4 mut Fc (FIG. 11). The volume of distributing was small, about 40-70 ml/kg. There was some evidence of non-linear PK for the 1-20 mg/kg does rage. The PK profiles of 2LM 20-4 and 2LM 20-4 mut Fc were comparable to that of Rituxan and TRU-015.

In conclusion, we demonstrated that humanized anti-CD20 SMIPs, in particular 2LM 20-4, showed B-cell deletion efficacy that is comparable, if not better, than Rituxan.

### Example 5

### In Vivo Studies

A. In Vivo Evaluation of CD20-specific 018011, Against Subcutaneous or Systemically Disseminated Human B-Cell Lymphoma Xenografts

Preclinical anti-tumor efficacy of 018011 against human B-cell lymphoma xenografts grown in nude mice was examined. 018011 inhibited growth of established subcutaneous B lymphoma xenografts and caused regression of established B-cell lymphoma xenografts in mice. There was no clear dose-response relationship in the anti-tumor activity of 018011. In a mouse model of disseminated B-cell lymphoma, when administered early in the disease process, 018011 protected scid mice with systemically disseminated B-cell lymphoma from hind limb paralysis and death.

Test and Control Molecules

CD-20 Binding Molecules

018011 (3.1 mg/ml dissolved in 20 mM sodium phosphate, 240 mM sucrose, pH 6.0 or 4.09 mg/ml, dissolved in 10 mM histidine, 5% sucrose, pH 6.0) and TRU-015 was stored at −80° C. Rituximab (RITUXAN®) was obtained from MedWorld Pharmacy (Chestnut Ridge, N.Y.). Drugs were diluted in phosphate buffered saline before use.

Cell Lines

The B-cell lymphoma (BCL) line Ramos (CRL-1596) was obtained from the American Type Culture Collection (ATCC, Manassas, Va.). The diffuse large B-cell lymphoma line WSU-DLCL2 (ACC-575) was obtained from Deutsche Sammlung von Mikroorganismen and Zellkulturen GmbH (DSMZ Braunschweig, Germany). Cells were determined to be mycoplasma free by a DNA fluorochrome staining assay (Bionique Testing Laboratories, Saranac Lake, N.Y.). Cells were maintained in RPMI 1640 medium supplemented with 10% fetal bovine serum (FBS), 10 mM HEPES (N-2-hydroxyethylpiperazine-N′-2-ethanesulfonic acid), 1 mM sodium pyruvate, 0.2% glucose, penicillin G sodium (100 U/ml), streptomycin sulfate (100 μg/m), and L-glutamine (2 mM). Before use, viable cells were isolated by centrifugation (30 min at 1000×g) using a Lymphoprep (Axis Shield PoC AS, Oslo, Norway) density gradient.

Animals

Female, BALB/c and nu/nu (nude) mice (18-23 g) and CB17 scid male mice (18-23 g) were obtained from Charles River Laboratories, Wilmington, Mass. All mice were housed in micro isolator units and provided with sterile food and water ad libitum throughout the studies.

Subcutaneous BCL Xenografts

Female, athymic (nude) mice were implanted with Ramos or WSU-DLCL2 subcutaneous xenografts. An additional group of mice were exposed to total body irradiation (400 rads) to further suppress their residual immune system before tumor implantation of Ramos BCL. Mice were injected with 1×10⁷ Ramos cells or 5×10⁶ WSU-DLCL2 cells suspended in Matrigel (Collaborative Biomedical Products, Belford, Mass., diluted 1:1 in RPMI 1640 medium) in the dorsal, right flank. When the tumors reached an appropriate mass (usually >100 mg), they were staged to maximize uniformity of the tumor mass prior to the administration of therapy (n=8 to 10 mice/treatment group). Compounds were administered either iv or ip in sterile saline (0.2 ml/mouse). To account for the difference in molecular weight between 018011 and RITUXAN®, the dosage of each drug administered to mice was adjusted to allow for molar equivalence of each protein. Accordingly, the amount of RITUXAN® administered was approximately 137% of the amount of 018011 administered. Tumor length and width (in cm) was measured at least once a week and tumor mass was calculated by the following: tumor mass (g)=[0.5×(tumor width2)(tumor length)]. Mean (±sem) tumor mass for each treatment group was calculated and compared to the vehicle-treated group for statistical significance using ANOVA and subsequent pairwise comparison to the vehicle-treated group by a one-tailed t-test with the error term for the t-test based on the pooled variance across all treatment groups. Tumor mass values for each treatment group were recorded up to 100 days after the initiation of treatment or until the tumors grew to 15% of the body weight at which time these mice were euthanized according to institutional regulations. The number of tumor-free mice at the end of each study was recorded. Survival of mice was plotted and was determined by tumor mass; any mouse with a tumor mass ≧1.5 g was considered dead for the calculation of the survival plot even though mice were not killed until the tumor mass reached 15% of mouse body weight according to institutional guidelines.

Assessment of Anti-Tumor Efficacy Against Disseminated BCL

Male scid mice were injected intravenously with 3×10⁶ Ramos cells or 5×10⁶ WSU-DLCL2 cells in a volume of 0.2 ml in the tail vein. Dissemination and growth of the cells was allowed to occur over a period of 3 days (designated as the developing model), 6 days (designated the intermediate model) or 9 days (designated as the established model) prior to the initiation of drug therapy. Mice with disseminated disease (9 to 13 mice/treatment group) were administered vehicle (PBS), 018011, TRU-015 or RITUXAN® iv on designated days. Mice with disseminated disease were monitored daily for the presence of hind-limb paralysis or death for up to 100 days. Mice exhibiting hind-limb paralysis were euthanized by CO₂ asphyxiation according to institutional regulations.

The average survival time (days ±SD) was calculated for each group. The difference in survival distribution between groups was determined by using nonparametric methods comparing the survival distribution of the diseased mice. Multiple comparisons were performed using the rank transformation procedure. The rank transformation procedure consists of replacing the survival times with their ranks and applying the usual parametric F-test to the ranks. Multiple comparisons were performed using Tukey's method on the ranks. Tukey's method indicates the difference in survival times among mice with significance reported at the 0.05 level. The survival curves were constructed using the Kaplan-Meier method (J Am Stat Assoc 1958; 53:457-81).

Bone marrow cells from the femur were collected from some scid mice with disseminated Ramos BCL and evaluated for the expression of human CD19 or murine CD45 antigen by incubating with control FITC-labeled rat IgG2A, FITC-labeled mouse IgG1, FITC-labeled rat anti-mouse CD45 or FITC-labeled mouse anti-human CD19 (all FITC-labeled reagents from BD Pharmingen, San Diego, Calif.). Cells were pelleted, washed with PBS-1% BSA and then fixed with 1% formaldehyde. Samples were analyzed on a FACSort flow cytometer for the presence or absence of human CD19 expressing cells. The number of human CD19+ cells was displayed as a percent of the total number of cell population gated based on the forward and side light scatter properties characteristic of lymphoid cells identified by the expression of CD45 common leukocyte antigen.

The animals used in this study are listed in Table 5.

TABLE 6
Specifications of Mice Used

| Characteristics | Specifications |
|---|---|
| Species: | Mouse |
| Strain: | Balb/c, nu/nu, CB17 scid |
| Source: | Charlse River, Wilmington, MA |
| No. of Animals Per Group: | 8 to13 |
| Total No. of Animals: | 492 |
| Age and Sex: | 6 to 8 week old, Balb/c and nu/nu (female), |
| | CB17 scid (male) |
| Weight: | 18 g-25 g |
| Date Received: | 10 April 2006 |
| Acclimation Period: | 1 week minimum |

Results

Effect of 018011 on Ramos Subcutaneous Xenografts

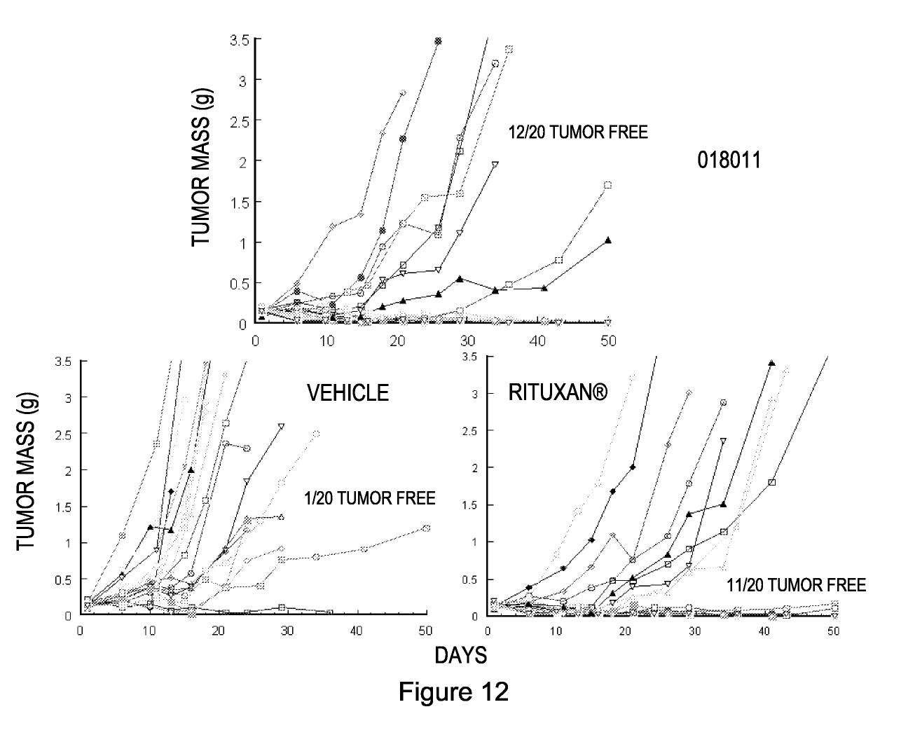

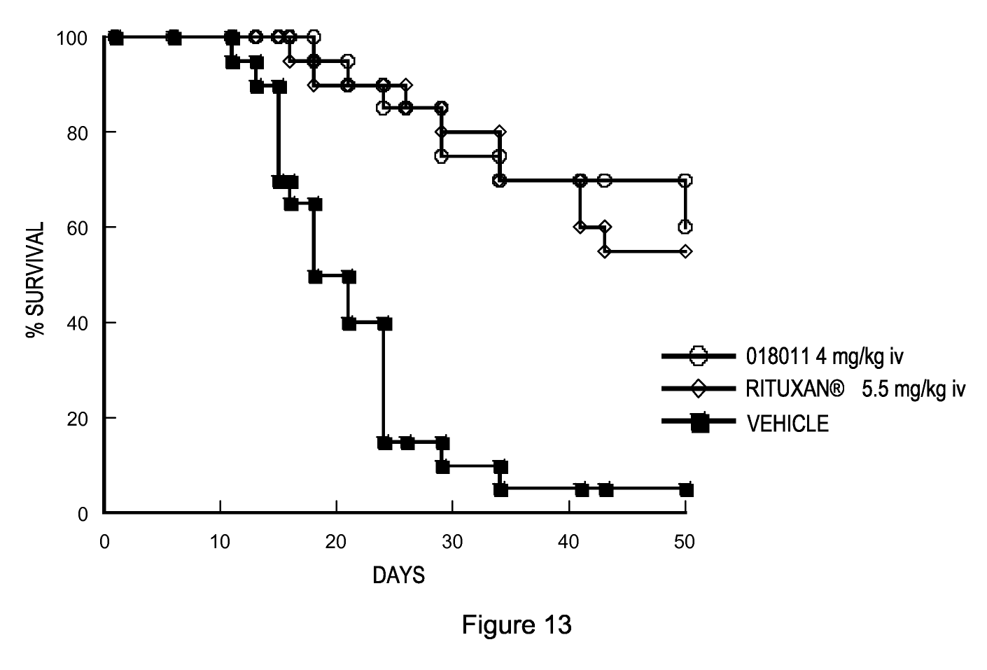

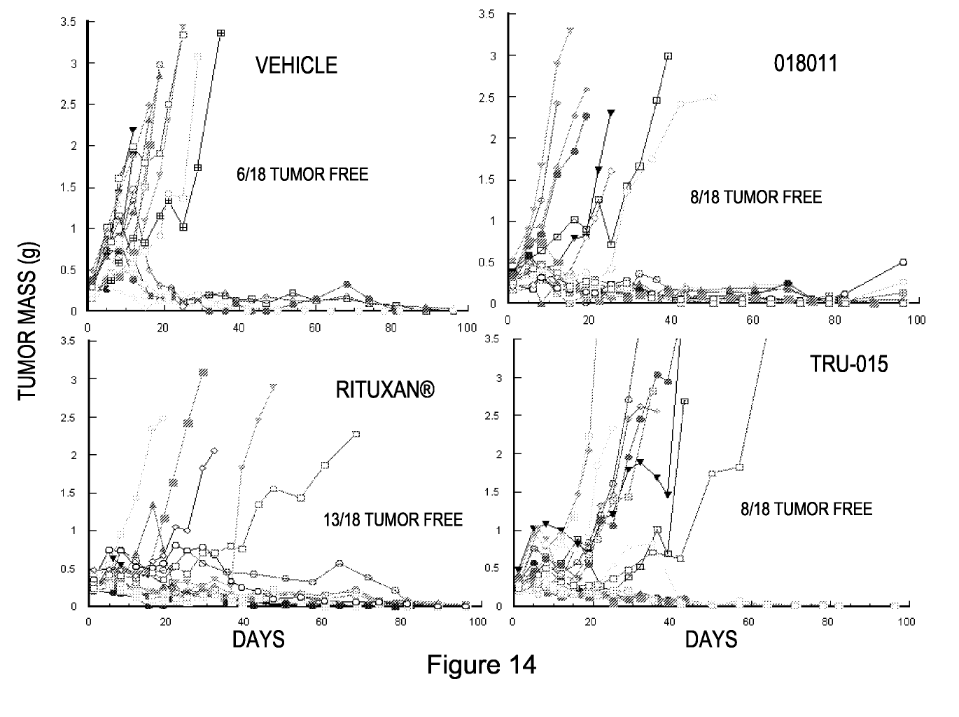

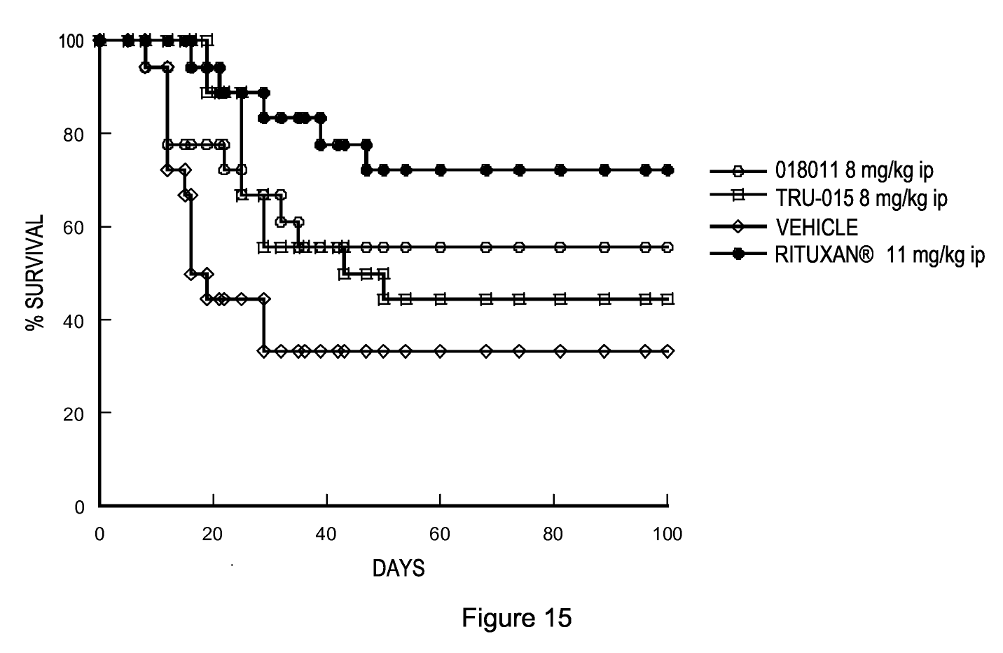

Vehicle, 018011 (4 mg/kg iv) or RITUXAN® (5.5 mg/kg iv) were administered at molar equivalent dosages to Balb/c mice with established Ramos xenografts. Individual mouse tumor mass over time is shown in FIG. 12 (results plotted are pooled from 2 separate studies) and survival (based on tumor mass <1.5 g) shown in FIG. 13. In another study conducted using nu/nu nude mice, vehicle, 018011 (8 mg/kg ip), TRU-015 (8 mg/kg ip) or RITUXAN® (11 mg/kg ip) were administered at molar equivalent dosages to nu/nu mice with established Ramos xenografts.

Dose responsive activity of 018011 and RITUXAN® was examined in the Ramos xenograft model established in Balb/c mice and of 018011, RITUXAN® and TRU-015 in the WSU-DLCL2 xenograft model established in nu/nu mice (Table 7). In both studies all compounds were administered as 5 doses every other day (excluding weekends) by the ip route of drug administration. The number of tumor free mice was recorded (day 50 for the Ramos study and day 100 for the WSU-DLCL2 study).

TABLE 7
Effect of 018011, TRU-015 and Rituximab on Ramos and
WSU-DLCL2 Xenografts

| | | Ramos | | WSU-DLCL2 |
|---|---|---|---|---|
| | | Day 50 | | Day 100 |
| | | Tumor Free Mice | | Tumor Free Mice |
| | Dose | (tumor free/ | Dose | (tumor free/ |
| Treatment | (mg/kg ip) | total mice) | (mg/kg ip) | total mice) |
| Vehicle | | 1/10 | | 3/10 |
| 018011 | 2 | 3/10 | 1 | 7/9 |
| | 4 | 0/10 | 2 | 2/9 |
| | 8 | 5/10 | 4 | 7/8 |
| | 16 | 3/10 | 8 | 6/8 |
| Rituximab | 2.8 | 1/10 | 1.4 | 6/8 |
| | 5.5 | 3/10 | 2.5 | 4/8 |
| | 11 | 3/10 | 5 | 7/8 |
| | 22 | 3/10 | 11.6 | 5/8 |
| TRU-015 | | | 1 | 4/8 |
| | | | 2 | 4/9 |
| | | | 4 | 6/8 |
| | | | 8 | 3/8 |

Effect of 018011, TRU-015 and Rituximab on Disseminated Lymphoma in Scid Mice

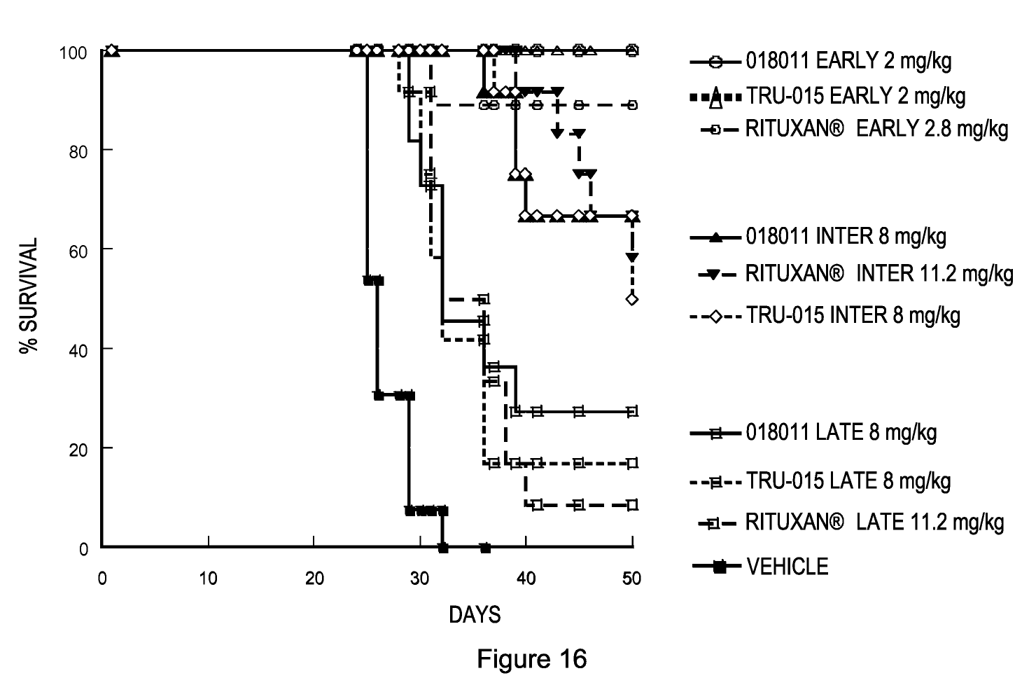

The ability of 018011, TRU-015 and RITUXAN® to prolong survival of CB17 scid mice with systemically disseminated B-lymphoma was evaluated. In this model, intravenous injection of CD19+ CD20+ Ramos B-lymphoma cells allows their rapid dissemination into various organs including the central nervous system, eventually causing hind-limb paralysis and death (Clin Can Res 2004; 10:8620-9). Compounds were administered iv in scid mice with disseminated Ramos B-lymphoma either during the developing (early) phase (therapy initiated 3 days post-dissemination of Ramos cells), during the intermediate phase of the disease (therapy initiated day 6 after tumor cell dissemination) or the established (late) phase (therapy administered 10 days post-dissemination) of the disseminated disease. As shown in FIG. 16 the administration of equimolar doses of 018011, TRU-015 or RITUXAN® (2 mg/kg, 2 mg/kg and 2.8 mg/kg, respectively) during the developing phase (beginning day 3) of the disseminated disease resulted in a significant protection against the developing disseminated disease. Delaying the administration of 018011, TRU-015 or RITUXAN® to 10 days post-dissemination resulted in a significant loss of the protective activity of each protein. During the established phase of the disease, each compound was given for 5 doses (as opposed to 3 doses in the developing phase and 4 doses in the intermediate phase) and at 4 times the dosage (8 mg/kg, 8 mg/kg and 11.2 mg/kg for 018011, TRU-015 and RITUXAN®, respectively) as was administered in the developing phase. Even at these higher and more prolonged dosages, the activity of each of the compounds in the established disease was significantly less than that during the developing phase of the disseminated model. These results suggest that 018011, TRU-015 and RITUXAN® are more efficacious against the developing disease than the established disease in the Ramos disseminated disease model.

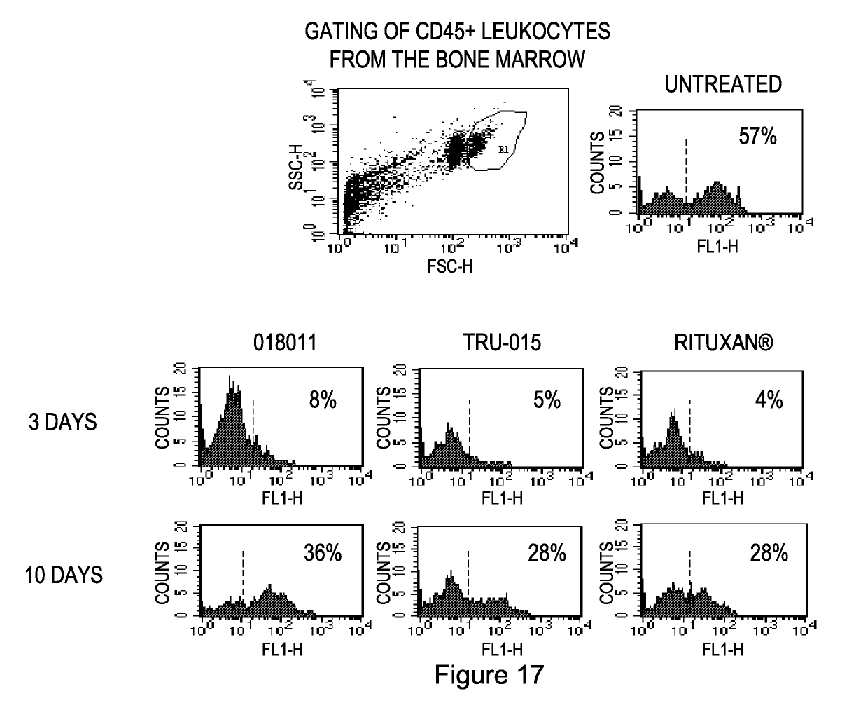

Bone marrow collected from mice with Ramos disseminated disease was examined for the presence of disseminated human CD19+ Ramos cells. Majority of the bone marrow-derived lymphoid cells from vehicle-treated mice with disseminated disease expressed human CD19 antigen indicative of the presence of human lymphoid cells in the bone marrow. Treatment with 018011, TRU-015 or RITUXAN® during the early phase (beginning 3 days post-BCL dissemination) of the disseminated disease process reduced the percentage of human CD19+ cells in the bone marrow to <10% (FIG. 17). None of these mice in the early-treatment group presented with hind-limb paralysis. Close to 30% of lymphoid cells isolated from the bone marrow of mice treated during the established phase (beginning 10 days post-BCL dissemination) of the disease process expressed human CD19 antigen. Mice in this late treatment group experienced hind-limb paralysis. The expansion of human B-lymphoma cells in the bone marrow may be an indication of the degree of disease progression in these diseased mice.

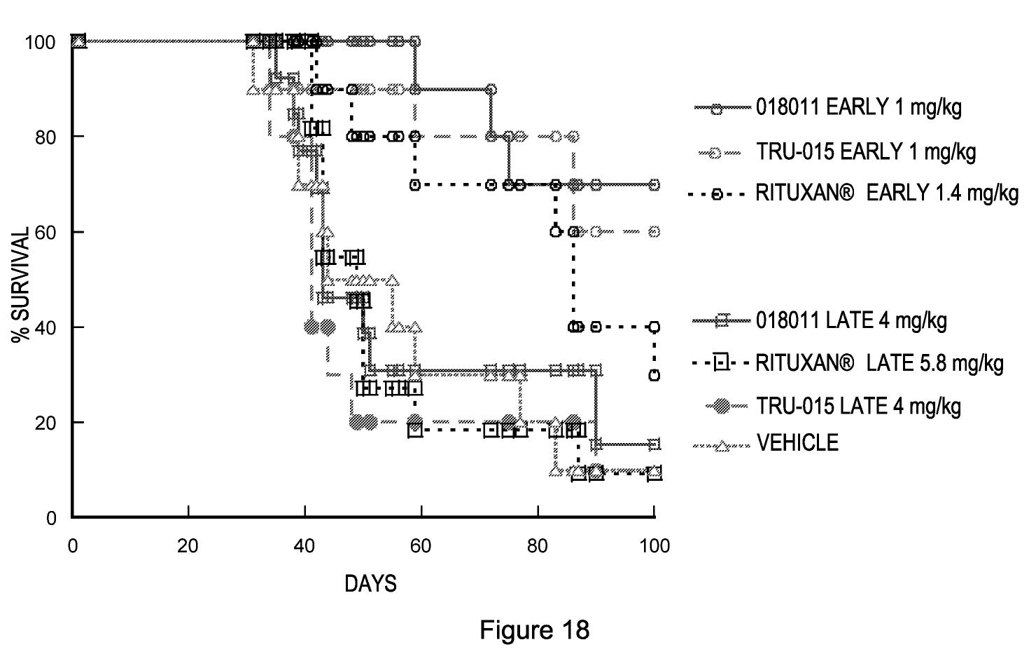

The effect of 018011, RITUXAN®, and TRU-015 was investigated in the WSU-DLCL2 diffuse large B-cell lymphoma disseminated disease model. Therapeutic proteins were administered iv beginning on day 3 post-tumor cell injection for a total of 3 doses for the developing model and beginning day 10 for a total of 5 doses for the established model. Proteins were administered at equivalent molar dosages. 018011 significantly (p<0.05 vs vehicle-treated mice) protected mice against the tumor cell-induced hind-limb paralysis (FIG. 18) while the effect of TRU-015 approached statistical significance (p=0.073 vs vehicle-treated mice) when administered early in the disease process (developing phase). The effect of RITUXAN® was not significant in the developing phase nor was the effect of any of the 3 proteins significant when administered during the established phase of the disease. These results are similar to those observed in the Ramos disseminated model when compounds were administered during the established phase of the disease.

Effect of Intravenously and Intraperitoneally Administered 018011 on Ramos Subcutaneous Xenografts

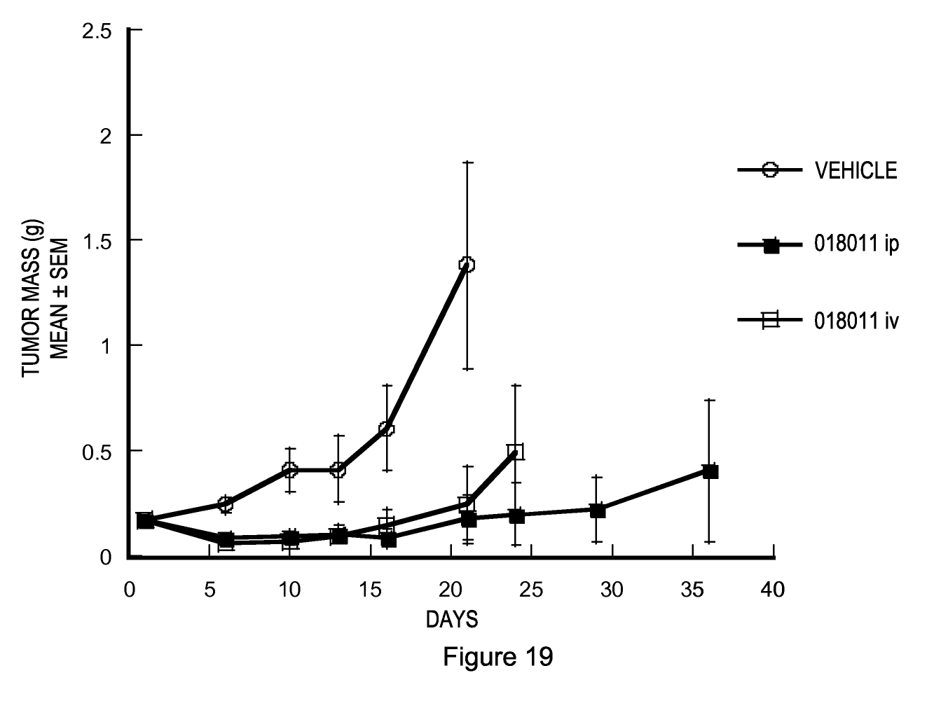

The anti-tumor activity of 018011 (8 mg/kg) was compared after iv and ip drug administration. 018011 was given 5 times every other day after tumor staging (Balb/c mice) and its anti-tumor activity was monitored. Both routes of drug administration significantly (p<0.05) inhibited tumor growth (FIG. 19). The anti-tumor activity of the 018011 administered ip was maintained for a greater length of time than that of 018011 administered iv. This study demonstrates that 018011 administered either ip or iv is efficacious in inhibiting the growth of human B-cell lymphoma xenografts.

Effect of 018011 and Rituximab on Subcutaneous Xenografts in Irradiated and Non-Irradiated Nude Mice

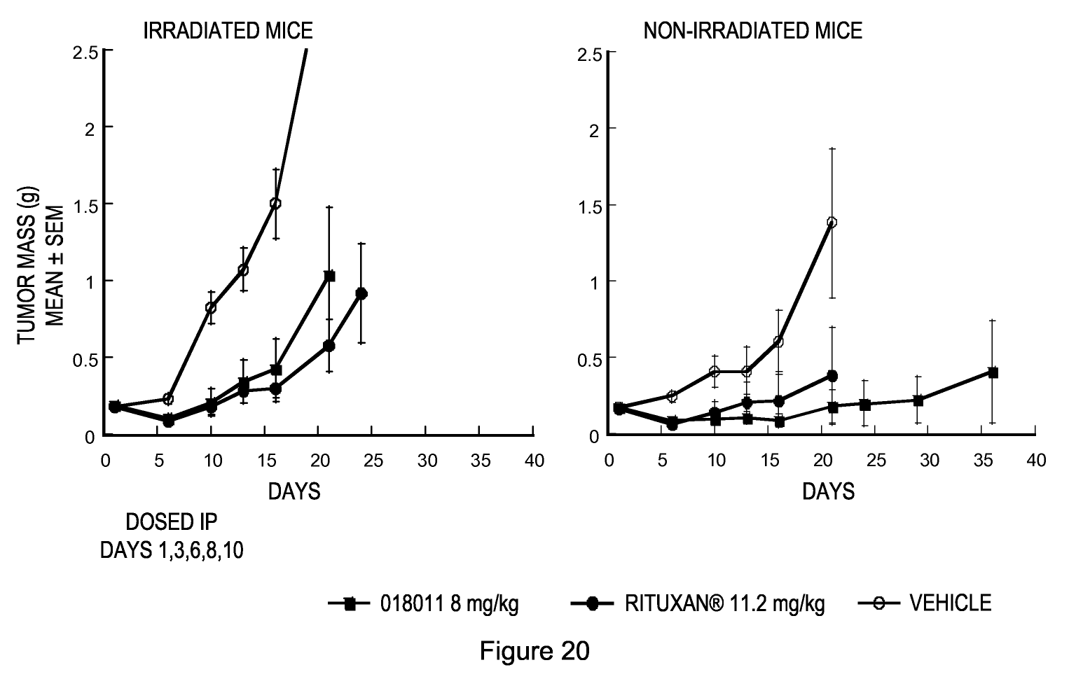

Gamma-irradiation can suppress the innate immune system facilitating establishment of tumor xenografts in immunocompromised nude mice. In light of the possibility that irradiation may also impact the effector cells capable of mediating the anti-tumor activity of therapeutic antibodies, the anti-tumor activity of 018011 and RITUXAN® against Ramos B-lymphoma xenografts was assessed in irradiated (4 Gy equivalent to 400 rads) or nonirradiated Balb/c nude mice. Ramos xenografts were established in both irradiated and non-irradiated mice. 018011 (8 mg/kg ip) and RITUXAN® (11.2 mg/kg ip) were each able to significantly (p<0.05 vs vehicle-treated mice) inhibit the growth of Ramos B-lymphoma xenografts in both irradiated and non-irradiated mice (FIG. 20). Tumors grew more rapidly and the inhibitory effect of each compound was not as robust in the irradiated mice as that observed in non-irradiated mice. These results suggest that irradiation of the host may negatively impact the therapeutic activity of immunotherapeutic agents such as 018011 or RITUXAN® that depend on the functional integrity of the effector cells of the immune system. The mechanism(s) by which irradiation effects the therapeutic activity of 018011 has not been investigated.

### CONCLUSION

018011 was active as an anti-tumor agent in preclinical models. It inhibited growth of established subcutaneous B lymphoma xenografts and protected mice with disseminated B-cell lymphoma when administered earlier rather than later in the disease process.

### B. Efficacy of HuCD20 SMIPS in an Established Ramos Tumor Xenograft Model in Nude Mice

The following experiments tested the efficacy of humanized CD20-specific SMIPS (HuCD20 SMIPS) in a model of established Ramos tumor xenografts in nude mice. The experiments were carried out in triplicate (referred hereon as (A), (B) and (C)). 7.5-8 week old Athymic Nude-Foxn1^{nu} mice (Harlan Livermore, Calif.) were used.

Establishment of Ramos Tumor Xenografts and Sorting into Treatment Groups

Ramos cells are a CD20⁺ human B-lymphoblastoid cell line derived from a Burkitt's lymphoma. Five million Ramos cells were injected subcutaneously into the flank of female athymic nu/nu mice. On day 6 post-tumor inoculation, palpable tumors were apparent in the majority of mice. The tumor-bearing mice were sorted into groups (n=8 per group; 2 cages of 4 mice for each group) with equivalent mean tumor volumes. The day of the sort was defined as Day 0 of the study. Tumors were measured with a calipers and tumor volumes were calculated using the formula: V=½[length×(width)²]. The baseline mean tumor volume for this experiment was 228 mm³, (A) and (B), or 227 mm³ (C); the median baseline tumor size was 228 mm³ (A), 233 (B), or 225 mm3 (C); and the range was 180-281 mm3 (A), 168-300 mm3 (B) or 157-300 mm3(C).

Reagents for in vivo use.

| | Source and | Concentration | Preparation for |
|---|---|---|---|
| Reagent | Lot No. | and endotoxin | injection |
| PBS | Gibco, | 1X concentration | NA |
| | 14190 | <0.03 EU/mL | |
| Human IgG | Sigma, | 10 mg/mL | Each protein |
| (huIgG) | I 4506 | endotoxin = 10 EU/mg | reagent listed at |
| 018008 | In house | 2.79 mg/mL | left was diluted to |
| | | endotoxin <0.25 EU/mg | 0.5 mg/mL in |
| 2Lm20-4 | In house | 2.79 mg/mL | PBS on day 0 of |
| | | endotoxin <0.25 EU/mg | the study. |
| 018011 | In house | 3.1 mg/mL | |
| | | endotoxin <0.25 EU/mg | |
| TRU-015 | In house | 10.2 mg/mL | |
| | | endotoxin <0.05 EU/mg | |

Blinding Protocol

The PBS and protein (drug) solutions were prepared in similar volumes and the contents of the tubes were noted on removable labels. An investigator who was not treating or assessing the mice placed a color code on each tube and noted the code and identity of the tube contents in a laboratory notebook. The possibility of investigator bias is reduced, but not eliminated, with this design because investigators performing the study were only partially “blinded” in that they did not know which treatment a particular group of mice was receiving, but did know that all the mice within a group of 2 cages belonged to the same group. The code was revealed at the end of the study; however, the investigator who was aware of the code was able to monitor the study results on an interim basis.

In Vivo Treatment

Mice were injected intravenously (IV) on days 0, 2, 4, 6, and 8 with 100 μg of human IgG, TRU-015, 018008, 018011, or 2Lm20-4 in a volume of 0.2 mL. PBS and drug solutions were color-coded as described above.

Monitoring and Endpoints

Mice were monitored daily by visual inspection. Weights were determined, and tumors were measured with a pair of calipers at least 3 times per week (M, W, F) by an observer blinded (see above) to the treatment groups. Tumor volumes were calculated as described above. Tumor volumes on the last day that all mice in all groups were alive were also expressed in terms of tumor volumes relative to day 0, using the formula:

$$Relative tumor volume on day of interest = \frac{( \begin{matrix}volume on day of interest - \\ volume on day 0\end{matrix} )}{volume on day 0}$$

Weight and tumor monitoring days were changed to once a week during times when the mice remaining in the study had no palpable tumors. Mice were sacrificed if the tumors reached more than 1500 mm³. Note that death is not an endpoint in the tumor protocols, and unless noted otherwise, “survival” of a mouse was determined by the time of its sacrifice.

The study was ended at day 90.

Statistical Analyses

All statistical analyses were performed using GraphPad Prism software. Significant differences in mean tumor volumes or mean relative tumor volumes on day 8 were determined using a using a one-way ANOVA with Dunnett's multiple comparison post-test (for comparison with huIgG control) and Tukey's multiple comparison post-test (for all other pairwise comparisons). Significant differences in survival of mice over time were determined using Kaplan-Meier survival analysis with a log-rank test for comparing survival curves. Significant differences in the incidence of tumor-free mice at the end of the observation period were determined using Fisher's exact test. p values <0.05 were considered significant.

Results

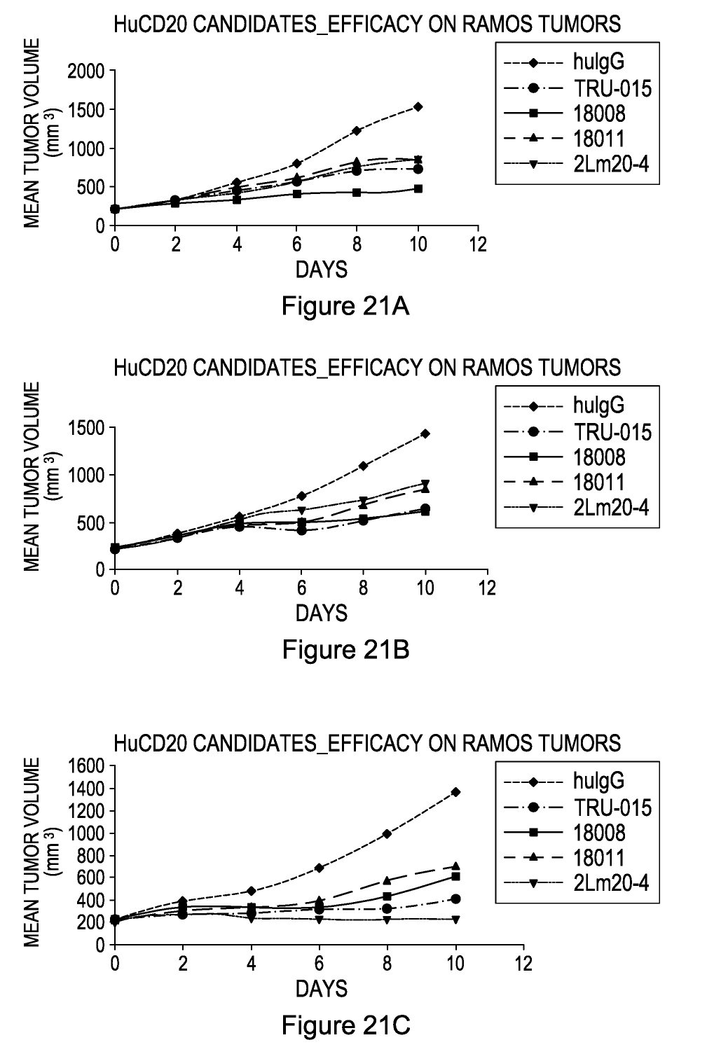

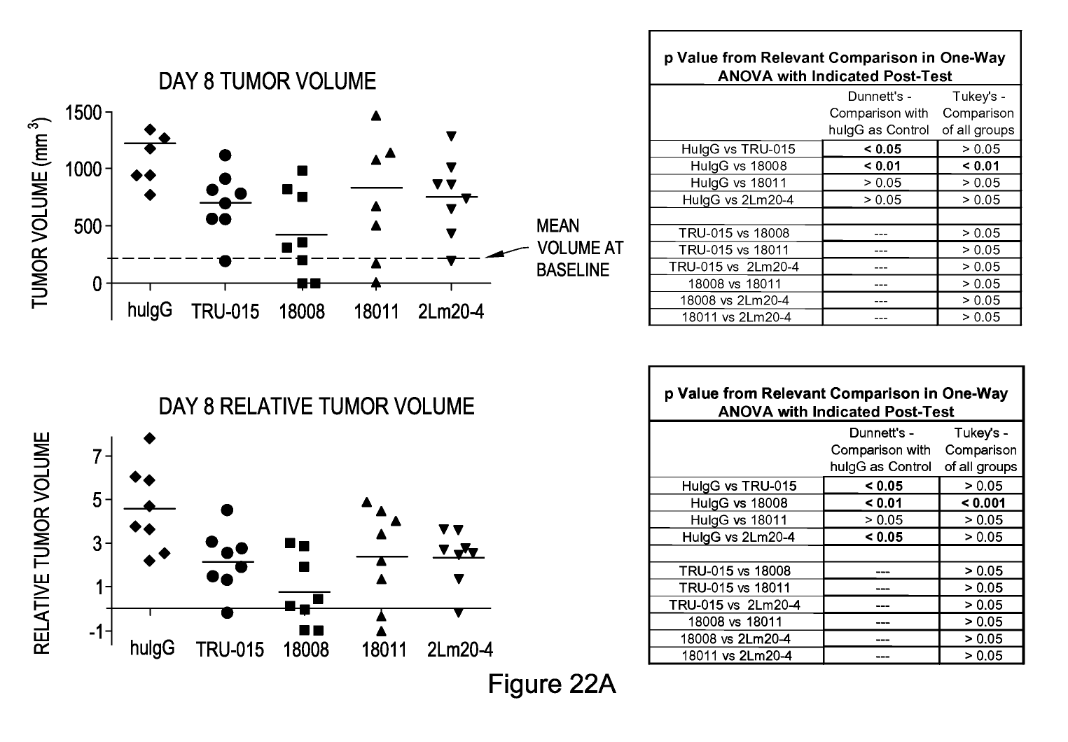

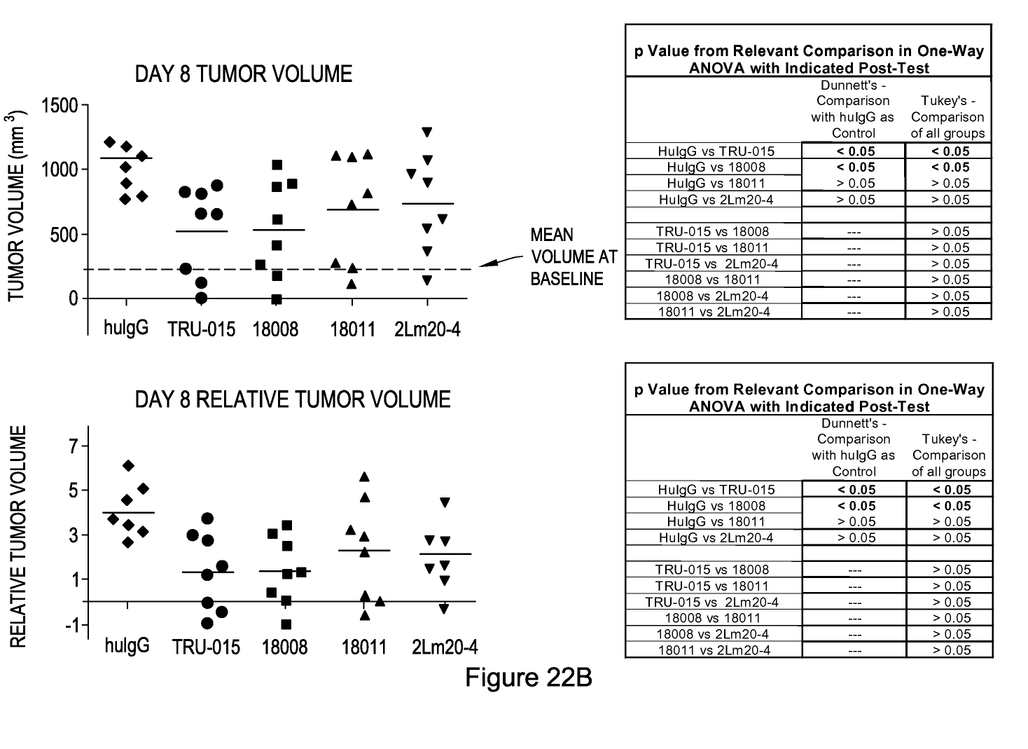

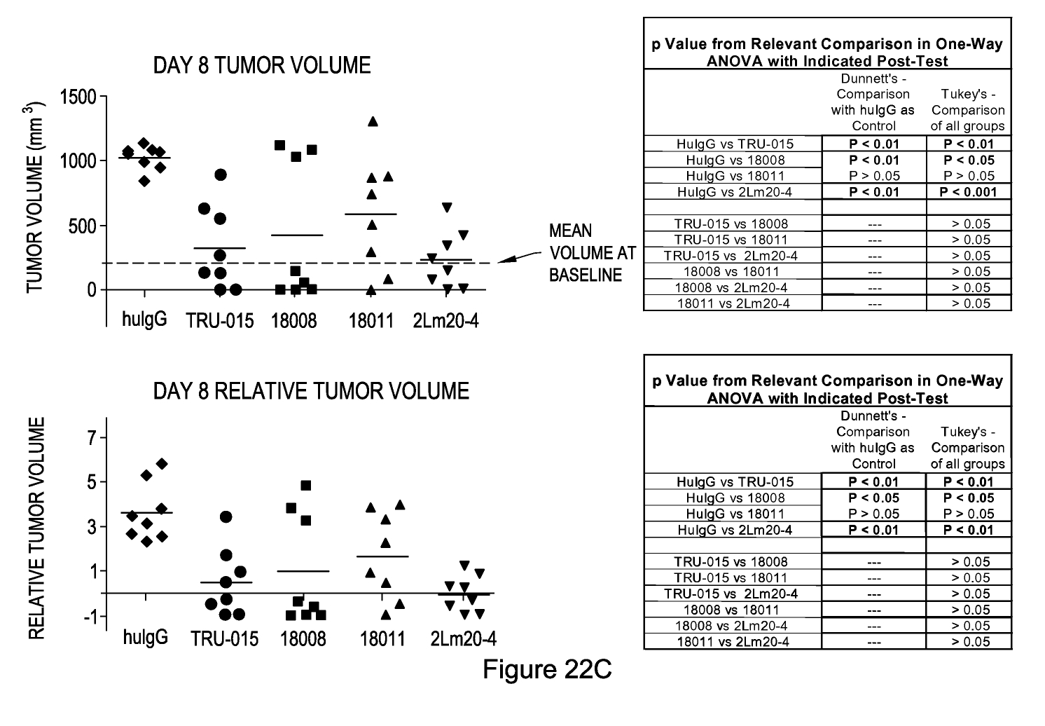

Treatment of mice with TRU-015 or with any of the three HuCD20 SMIPS (018008, 018011, and 2Lm20-4) resulted in a slowing of tumor growth relative to controls and/or reduction in tumor volume relative to the baseline measurements (FIGS. 21-23). The mean tumor volume and the mean relative tumor volume of the huIgG-treated group differed significantly from the TRU-015, 018008 and 2Lm20-4 ((C) only) treated groups at day 8, which was the last day at which all mice were alive (FIGS. 22A-22B). There were no significant differences in mean tumor volumes or relative tumor volumes between the huIgG treated group and any other HuCD20 SMIP treated group or between any two HuCD20 SMIP treated groups. Mice were sacrificed starting on day 8; therefore comparisons of tumor volumes were not made at the later time points.

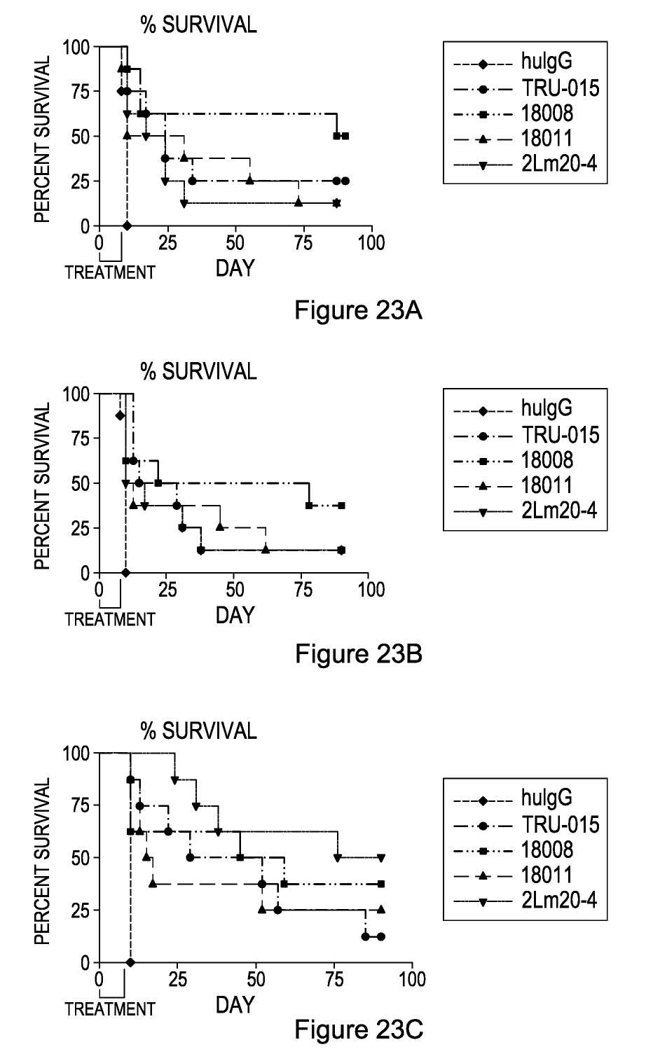

Mice were sacrificed when the tumor volume reached the limits mentioned above. No mice were found dead and none were sacrificed due to extreme weight loss, tumor ulceration or impaired mobility, thus the “survival” time was another measure of the rate of tumor growth. As shown in FIG. 23 and summarized in Table 8, the median survival time in the huIgG control group was 10 days. In contrast, median survival times were increased significantly relative to the control group in each of the other groups of mice. The median survival times of the mice in the TRU-015, 018008, 018011, and 2Lm20-4 treatment groups were 24.0, 88.5, 20.5 and 20.5 days (A), 22.0, 50.0, 11.5 and 13.5 days (B), or 40.5, 52, 16 and 83 days (C), respectively. There was no significant difference in the median survival times between any two of these groups (Table 8).

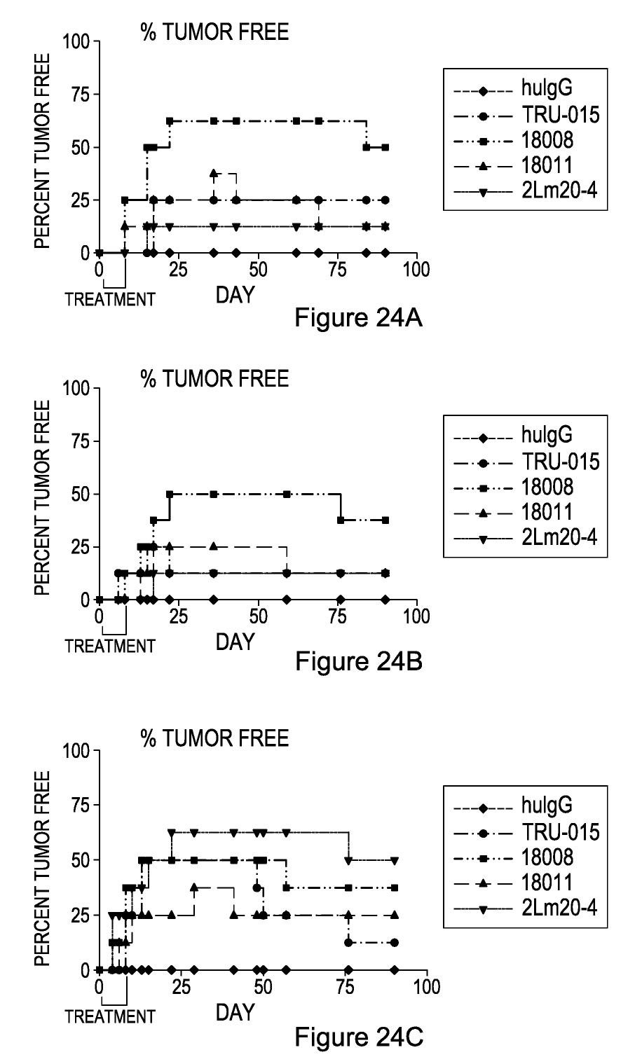

None of the 8 huIgG-treated mice were alive at the end of the 90 day observation period and thus the tumor-free incidence of this group at day 90 was 0/8 (FIG. 24 and Table 8). The incidence of tumor-free mice at the end of the observation period was 2/8 (25%) (A) or 1/8 (12.5%) (B) and (C), in the TRU-015 treated group; 4/8 (50%) (A), 3/8 (37.5%) (B) and (C), in the 018008 treated group; 1/8 (12.5%) (A) and (B), or 2/8 (25%) (C), in the 018011 treated group; and 1/8 (12.5%) (A) and (B), or 4/8 (50%) (C), in the 2Lm20-4 treated group. There was no significant difference in the incidence of tumor-free mice between the huIgG control group and any of the treatment groups, or between any two other groups of mice (Table 8 and 9).

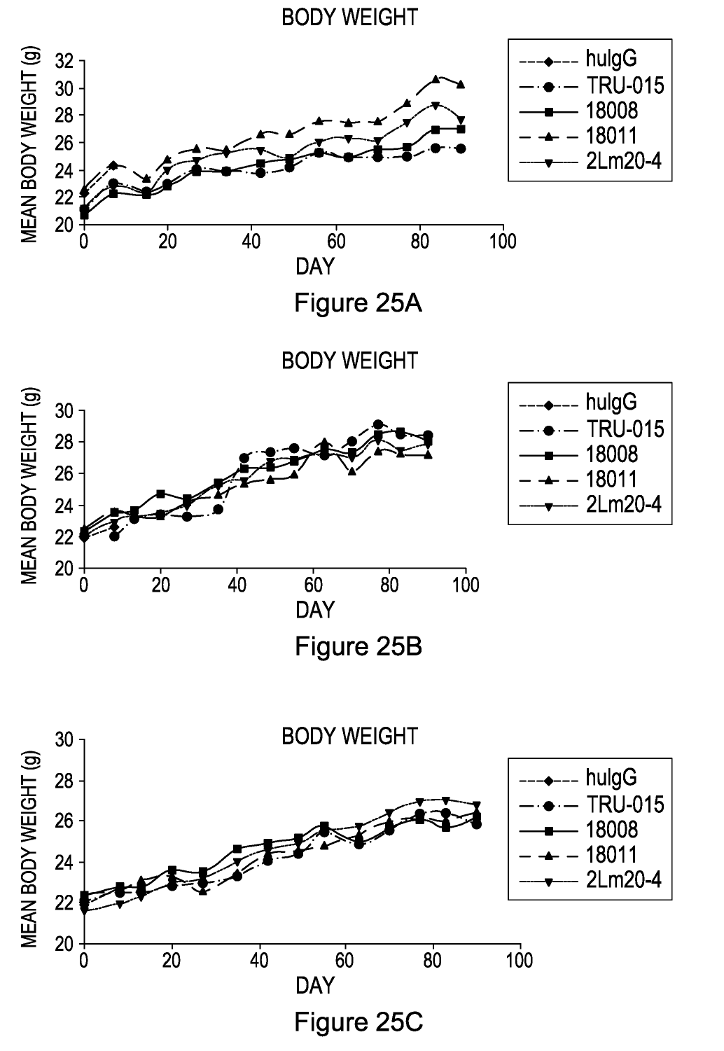

No overt signs of toxicity or weight loss were observed in any of the treatment groups (FIG. 25).

TABLE 8
Median Survival Time and Incidence of Tumor-Free Mice
at the end of the Observation Period

| | Median | | | p Value from |
|---|---|---|---|---|
| | Survival | p Value From | Tumor-Free | Fisher's Exact |
| Treatment Group | Time (days)^{a} | Log Rank Test^{b} | Mice at Day 90^{c} | Test^{b} |
| HuIgG | 10 | — | 0/8 (0%) | — |
| (200 μg) | | | | |
| TRU-015 | 24 (A) | 0.0023 (A) | 2/8 (25%) (A) | 0.4667 (A) |
| (200 μg) | 22 (B)^{d} | 0.0001 (B) | 1/8 (12.5%) (B) | 1.0000 (B, C) |
| | 40.5 (C) | 0.0006 (C) | 1/8 (12.5%) (C) | |
| 018008 | 88.5 (A) | 0.0007 (A) | 4/8 (50%) (A) | 0.0769 (A) |
| (200 μg) | 50 (B) | 0.0079 (B) | 3/8 (37.5%)(B, C) | 0.2000 (B, C) |
| | 52 (C) | 0.0090 (C) | | |
| 018011 | 20.5 (A) | 0.0470 (A) | 1/8 (12.5%) (A, B) | 1.0000 (A, B) |
| (200 μg) | 11.5 (B) | 0.0198 (B) | 2/8 (25%) (C) | 0.4667 (C) |
| | 16 (C) | 0.0006 (C) | | |
| 2Lm20-4 | 20.5 (A) | 0.0061 A) | 1/8 (12.5%) (A, B) | 1.0000 (A, B) |
| (200 μg) | 13.5 (B) | 0.0198 (B) | 4/8 (50%) (C) | 0.0769 (C) |
| | 83 (C) | 0.0001 (C) | | |

^{a}“Survival” of a mouse was determined by the time of its sacrifice due to tumor growth. No mice were found dead or sacrificed for other reasons. The study ended at Day 90.
^{b}Each group was compared with the HuIgG treated group. For other comparisons, see Table 9 below.
^{c}“Tumor-free” mice had no palpable tumors. The absence of tumor cells was not confirmed by histology.
^{d}Bold-faced values are significantly different from those of HuIgG control group.

TABLE 9
p Values for Comparison of Survival Curves and Tumor-Free
Incidence Between the TRU-015 and HuCD20 SMIP Treated
Groups

| | P Values for Indicated Comparisons^{a} | |
|---|---|---|
| | Log rank test | Fisher's exact test |
| | (comparison of | (comparison of tumor-free |
| HuCD20 SMIP Groups | survival curves) | incidence) at day 90 |
| TRU-015 vs. 018008 | 0.3594 (A) | 0.6084 (A) |
| | 0.4405 (B) | 0.5692 (B) |
| | 0.4452 (C) | 0.5692 (C) |
| TRU-015 vs. 018011 | 0.6727 (A) | 1.0000 (A, B, C) |
| | 0.8367 (B) | |
| | 0.9165 (C) | |
| TRU-015 vs. 2Lm20-4 | 0.4535 (A) | 1.0000 (A, B) |
| | 0.7089 (B) | 0.2821 (C) |
| | 0.1069 (C) | |
| 018008 vs. 018011 | 0.0806 (A) | 0.2821 (A) |
| | 0.2421 (B) | 0.5692 (B) |
| | 0.6302 (C) | 1.0000 (C) |
| 018008 vs. 2Lm20-4 | 0.1225 (A) | 0.2821 (A) |
| | 0.2421 (B) | 0.5692 (B) |
| | 0.5243 (C) | 1.0000 (C) |
| 018011 vs. 2Lm20-4 | 0.7614 (A) | 1.0000 (A, B) |
| | 0.7766 (B) | 0.6084 (C) |
| | 0.1274 (C) | |

^{a}See legend to Table 8 for information on groups.

On study Day 0, nude mice bearing palpable Ramos tumors were sorted into treatment groups (n=8/group) such that the mean tumor volume for each group was equivalent. Mice were treated IV on days 0, 2, 4, 6, and 8 with 100 μg of human IgG, TRU-015, 018008, 018011, or 2Lm20-4. Tumors were measured on the indicated days with a caliper and tumor volume was calculated using the formula: V=½[length×(width)2]. Once an animal was taken out of the study due to tumor volume exceeding specified limits, the value for the last tumor volume was carried forward. Results are shown only through day 10, when the last control mice were sacrificed.

Results are shown in terms of tumor volume of individual mice on day 8 (the last time point in which all mice were alive) (A) or relative tumor volume of individual mice on day 8 relative to day 0 (B). Significant differences among groups were determined using a one-way ANOVA with Dunnett's multiple comparison post test (for comparison with huIgG treated controls) and Tukey's multiple comparison post test (for all other pairwise comparisons); p values for all pair wise comparisons are indicated.

Mice were treated and monitored, and tumor volumes were determined as described in the legend to FIG. 21. Tumor volumes were determined at least 3 times a week (M W F) with the exception that monitoring was switched to once per week during time periods when all mice remaining in the study had no palpable tumors. Mice were sacrificed when tumor volumes reached more than 1500 mm3 (or 1200 mm3 on Fridays). No mice were found dead or sacrificed for other reasons.

### C. Intratumoral Accumulation of Humanized CD20 Binding Molecule

Intratumoral accumulation of 018011 in subcutaneous human B-cell lymphoma xenografts established in nude mice was evaluated by indirect immunofluorescence analysis (IFA) and flow cytometry and compared with that of RITUXAN®, a benchmark CD20-specific antibody therapeutic. Both agents were administered as a single intravenous dose. As assessed by flow cytometry, RITUXAN® bound to a greater degree to Ramos or WSDLCL2 cells in vitro. This difference was not apparent by IFA analysis. IFA analysis of WSU-DLCL2 subcutaneous xenografts (agents administered 0.5 mg iv) demonstrated that staining for 018011 tended to be more diffuse throughout the tumor while RITUXAN® staining appeared more punctuate and not as diffuse as that of 018011. Similar results were observed in Ramos xenografts when agents were administered at a dose of 1 mg/mouse and analyzed either 24 h or 96 h post administration. Flow cytometric analysis of lymphoma cells isolated from xenografts confirmed this observation.

Test and Control Articles

CD-20 Binding Molecules

018011 (3.1 mg/ml dissolved in 20 mM sodium phosphate, 240 mM sucrose, pH 6.0 or L37852-001, 4.09 mg/ml, dissolved in 10 mM histidine, 5% sucrose, pH 6.0) was stored at −80° C. Rituximab (RITUXAN®), trastuzumab (HERCEPTIN®), and cetuximab (ERBITUX®) were obtained from MedWorld Pharmacy (Chestnut Ridge, N.Y.). Drugs were diluted in phosphate buffered saline before use.

Cell Lines

The B-cell lymphoma line Ramos (CRL-1596) was obtained from the American Type Culture Collection (ATCC, Manassas, Va.). A diffuse large B-cell lymphoma line WSU-DLCL2 (ACC-575) was obtained from Deutsche Sammlung von Mikroorganismen and Zellkulturen GmbH (DSMZ Braunschweig, Germany). All cell lines were determined to be mycoplasma free by a DNA fluorochrome staining assay (Bionique Testing Laboratories, Saranac Lake, N.Y.). Each cell line was maintained in RPMI 1640 medium supplemented with 10% fetal bovine serum (FBS), 10 mM HEPES (N-2-hydroxyethylpiperazine-N′-2-ethanesulfonic acid), 1 mM sodium pyruvate, 0.2% glucose, penicillin G sodium (100 U/ml), streptomycin sulfate (100 μg/m), and L-glutamine (2 mM). Before use, viable cells were isolated by centrifugation (30 min at 1000×g) using a Lymphoprep (Axis Shield PoC AS, Oslo, Norway) density gradient.

Animals

Female, BALB/c, nu/nu (nude) mice (18-23 g) were obtained from Charles River Laboratories, Wilmington, Mass. All mice were housed in micro isolator units and provided with sterile food and water ad libitum throughout the studies.

The animals used in this study are listed in Table 10.

TABLE 10
Specifications of Mice Used

| Parameters | Specifics |
|---|---|
| Species: | Mouse |
| Strain: | Balb/c nudes |
| Source: | Charles River, Wilmington, MA |
| No. of Animals Per Group: | 1-4 |
| Total No. of Animals: | 26 |
| Age and Sex: | 6 to 8 week old, Balb/c nudes (female) |
| Weight: | 18 g-25 g |
| Date Received: | 05 Feb. 2007 |
| Acclimation Period: | 1 week minimum |

Immunofluorescence (IFA)

Ramos or WSU-DLCL2 cells were plated at 500,000 cells per well on Poly-D-Lysine 8 well culture slides (BD BioCoat cat #354632) and grown overnight. The following day, cells were incubated with 100 nM RITUXAN®, trastuzumab, or 018011 for 30 minutes at 37° C. in the presence of 5% CO₂. Cells were then fixed with 3.75% formaldehyde and 0.2M sucrose in PBS. Cells were re-hydrated by washing with PBS (3×5 minutes) and then incubating in 0.1 M glycine in PBS for 10 minutes. Cells were blocked with 3% BSA/PBS for 1 hour. All subsequent steps were conducted in the dark. Specific binding was analyzed using fluorescein (FITC) goat anti-human IgG, Fcγ fragment specific at 1:100 dilution in 1% BSA/PBS for 30 minutes at 37° C. (Jackson ImmunoResearch Labs cat# 109-095-008). Some slides were also stained with R-PE anti mouse CD31 (Caltag Laboratories). To separate the chamber and slide, chambers were soaked in 70% MeOH for 2 min taking care not to expose the cells. A drop of mounting media with DAPI counterstain (ProLong anti-fade reagent with DAPI, Invitrogen cat #P36931) was added to each chamber with a cover slip. Immunofluorescence was evaluated using a fluorescent microscope (Nikon Eclipse E400). To allow direct comparisons, all images were obtained using the same parameters (brightness, contrast, 40× magnification, etc.). Images were acquired with a Spot RT Slider (Diagnostics Instruments, Sterling heights, MI) digital camera and processed using Spot Advanced (version 4.0.9) digital software

Intratumoral Accumulation into Subcutaneous Xenografts

Female, athymic (nude) mice were injected with 1×10⁷ Ramos cells suspended in Matrigel (Collaborative Biomedical Products, Belford, Mass., diluted 1:1 in RPMI 1640 medium) in the dorsal, right flank. When the tumors reached an appropriate size, vehicle (PBS), 018011, RITUXAN®, trastuzumab (used as an isotope matched, non-binding control) or cetuximab (used as an isotope matched, non-binding control) were administered iv (0.2 ml/mouse) into the tail vein of the mouse. Tumors were excised after 24 or 96 h for analysis. Tumors were weighed at time of necropsy. Excised tumors were snap frozen in embedding medium (O.T.C. compound, Tissue-Tek cat#4583, Sakura Fintec Torrance, Calif.) and stored at −80° C. Four micron frozen sections were cut with a microtome (Tissue-Tek Cryo 2000). Sections were air dried for thirty minutes and stored at −80° C. Sections were then fixed and processed as described according to the IFA protocol (3.1).

Tumor Tissue Digestion and Flow Cytometry

Tumors were excised and minced into 1-2 mm pieces. Tumor pieces were digested with Type 4 collagenase treatment (Worthington, Lakewood, N.J.) by adding 2 ml of a 2 mg/ml stock to the tumor pieces for 30 min at 37° C. Cells were titerated, spun down to collect the cells, and then resuspended in fresh culture media. Binding to the dispersed tumor cells by 018011, RITUXAN® or trastuzumab was then assessed by flow cytometry as described in Example 1.

Results

Intratumoral Accumulation into Subcutaneous Xenografts

Mice with WSU-DLCL2 subcutaneous xenografts (tumor mass between 130 mg to 270 mg) were administered 018011, RITUXAN®, or trastuzumab (0.5 mg/mouse iv) and tumors were excised for IFA 24 h later. Staining for 018011 tended to be more diffuse than that of RITUXAN®. RITUXAN® staining appeared more punctuate and intense and not as diffuse as that of 018011. Sections were also stained for blood vessels (CD31). Neither agent appeared to accumulate around the vessels, both were able to diffuse deep into the tumor. Mice were also treated with a dose of 0.2 mg/mouse iv of 018011 and RITUXAN®. IFA of the tumors taken from the 0.2 mg treatment group demonstrated staining of both 018011 and RITUXAN® (data not presented) though neither agent was as intense or diffuse in its staining as was observed in the 0.5 mg treatment groups.

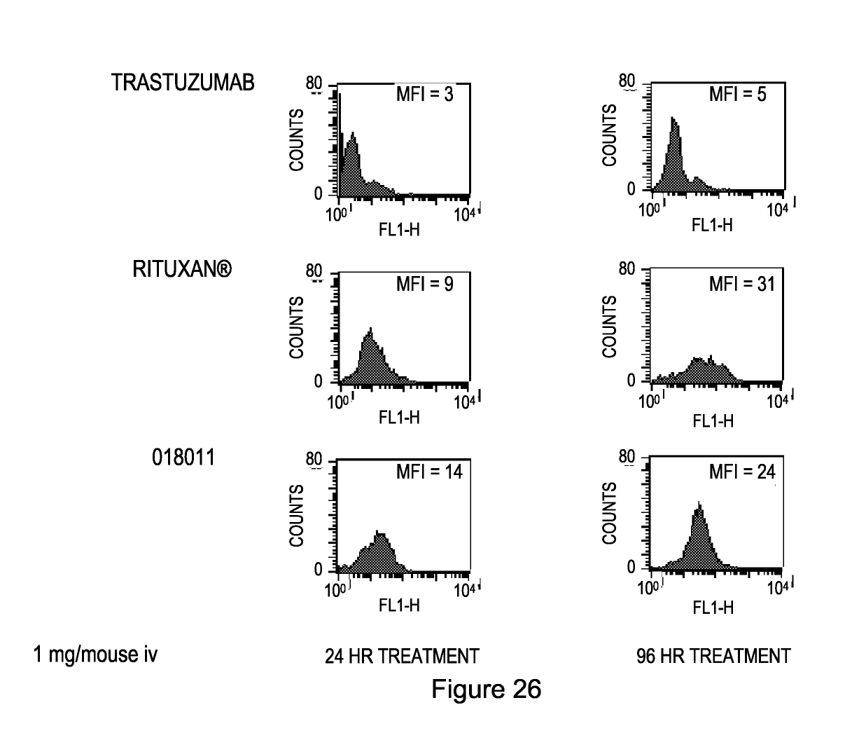

Mice with Ramos subcutaneous tumors (tumor mass between 100 mg and 400 mg) were treated with 018011, RITUXAN® or trastuzumab (all human IgG1) at 1 mg/mouse iv and tumors were excised at 24 and 96 h after the injection of the agents. Both 018011 staining intensity and RITUXAN® staining intensity appeared to be the same after either 24 or 96 h. Similar to the observations made using WSU-DLCL2 xenografts, 018011 staining appeared to be more diffuse than that of RITUXAN®, which was more punctuate and intense. Trastuzumab produced very minimal background staining. Part of each xenograft tissue used for IFA was digested at the time of excision and then the presence of 018011, RITUXAN®, or trastuzumab bound to xenografts-derived cells was analyzed by flow cytometry (FIG. 26). The flow cytometric profile suggested that both 018011 and RITUXAN® were detected bound on the cell surface of the xenograft-derived cells. Binding of 018011 and RITUXAN® was quantitatively similar. Cells from the xenografts isolated 96 hr after the iv injection of 018011 or RITUXAN® had higher levels of the CD20-binder than those isolated 24 hr after the same treatment. There was no detectable 018011 or RITUXAN® bound to xenografts-derived cells isolated 2 hr after the iv injection of either agent (data not presented). When the dose of each agent was reduced to 0.5 mg/mouse iv, it appeared that the staining intensity of 018011 was slightly enhanced in 2 separate studies (assessed at 24 h post dose) relative to RITUXAN®, though this was not quantifiable under the conditions of the study. Tumor mass was between 290 mg and 420 mg for mice used for generating results in and between 820 mg and 3000 mg for mice used in generating the results in. It appears, therefore, that tumor size did not significantly affect the tumor accumulation of either agent since results observed in were comparable while tumor masses were considerably larger in the second of the studies. When mice were treated with lower doses of either CD20 targeting agent (0.05, 0.1, or 0.2 mg/mouse iv), no 018011 was detectable while minimal staining of RITUXAN® was observed at the 0.1 and 0.2 mg dose groups (data not presented).

### Example 6

### Growth Inhibition

### Growth Inhibition Assay

In vitro growth of BCL was assessed using MTS, a vital dye (Promega, Madison, Wis.). This test depends on the conversion of MTS into a colored product by intact mitochondria from viable cells and is a reliable indicator of viable cells in culture. For each of the 6 BCL a calibration curve (cell number versus optical density of the colored product derived after approximately 2 h incubation time with MTS) was established to estimate an appropriate initial seeding density. Cells were then seeded in 96-multiwell dishes at a density of 10,000 to 50,000 cells per well depending on the cell line. After seeding, the cells were exposed to various concentrations of 018011, TRU-015 or RITUXAN® and, after a 96 hr incubation period, the viable cell number in each culture was determined.

The effect of crosslinking 018011 or RITUXAN® with anti-human IgG Fc antibody was assessed using propidium iodide (PI) exclusion measured by flow cytometry. Cell membranes of damaged cells allow PI to enter the cells and stain nuclear DNA whereas cell membranes of viable cells are impermeable to PI and thus PI cannot stain their nuclear DNA. To this end, thirty thousand BCL were plated in 96 well microtiter plates with increasing concentrations of 018011 or RITUXAN® followed by the addition of goat anti-human IgG Fc antibody (Jackson Immunoresearch) at either 1 or 10 μg/ml and incubation at 37° C. for 24 h. After the incubation, cell viability was measured flow cytometrically by PI exclusion. Data analysis was conducted using the CellQuest program (Becton-Dickinson).

Growth Inhibitory Effect of 018011 Against Human B-Lymphoma Cells

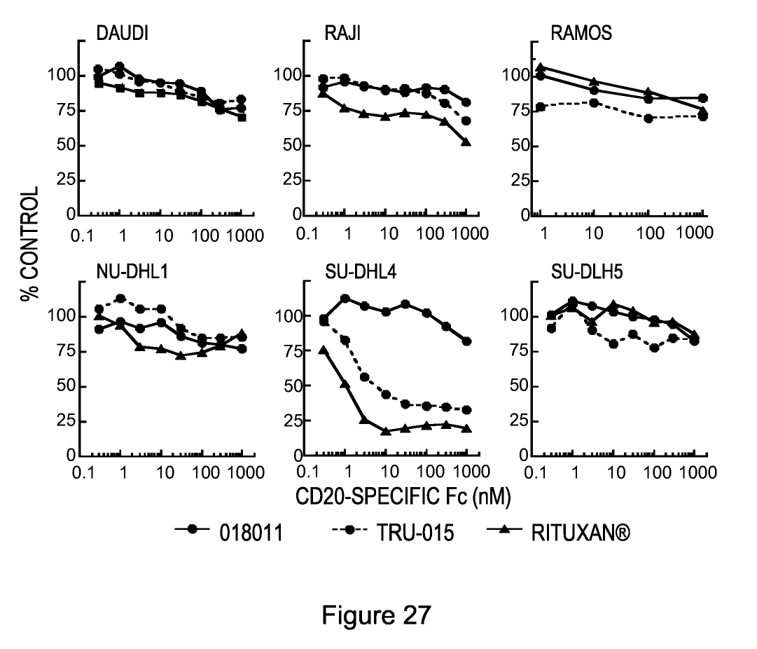

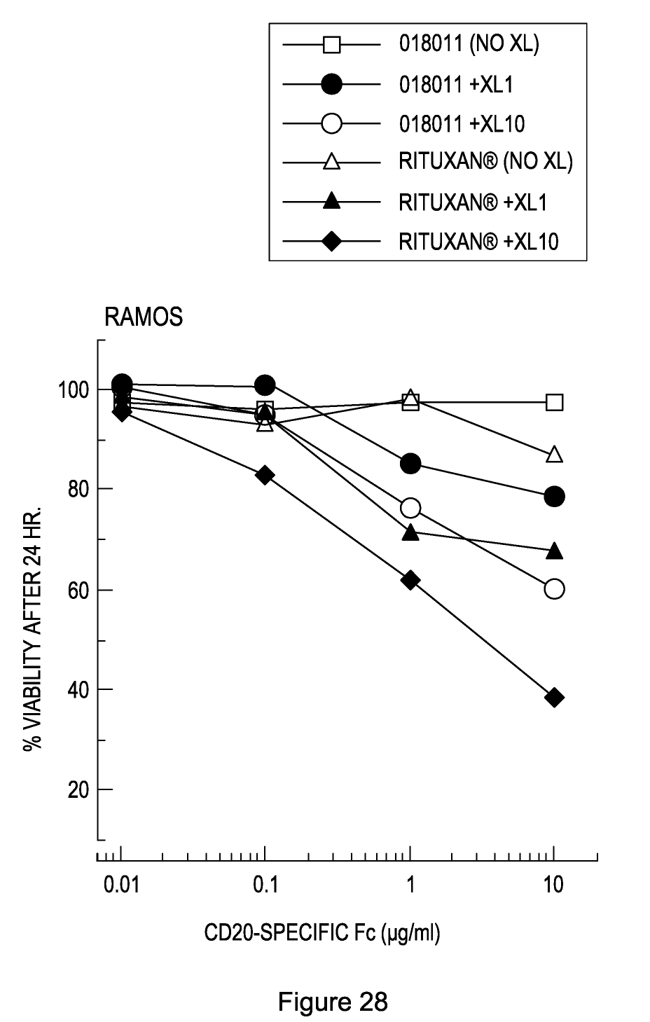

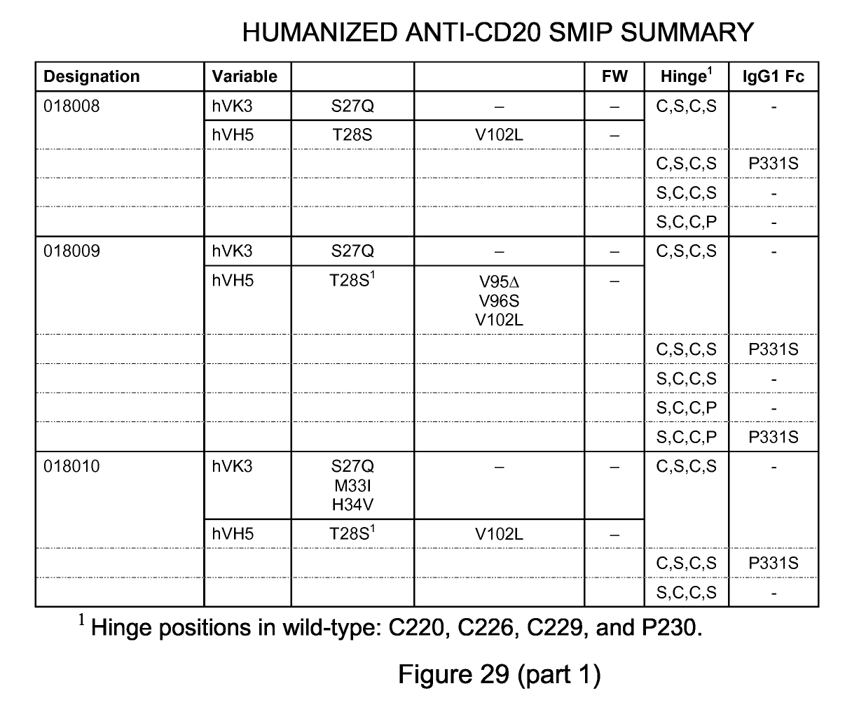

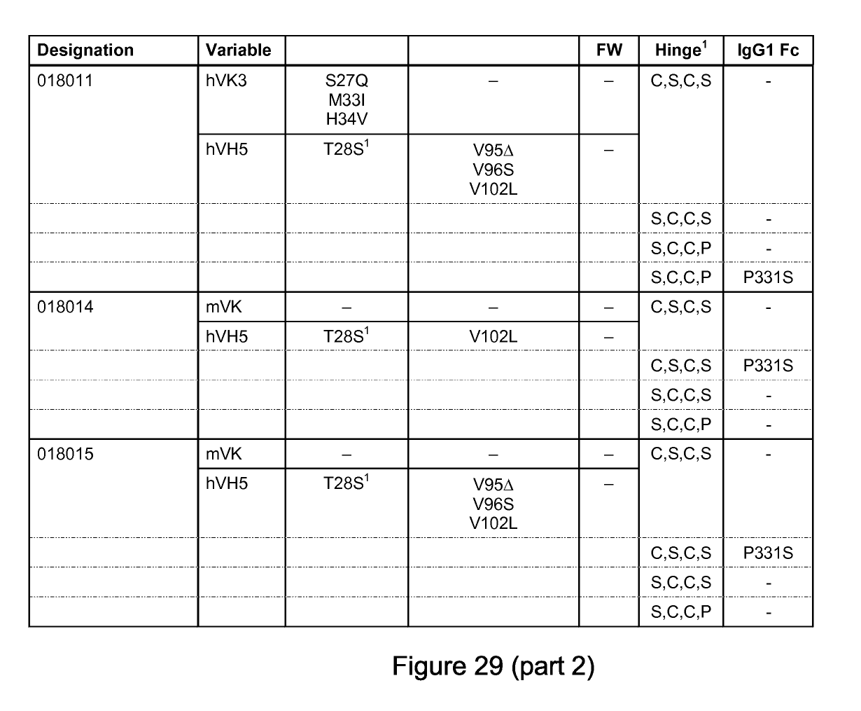

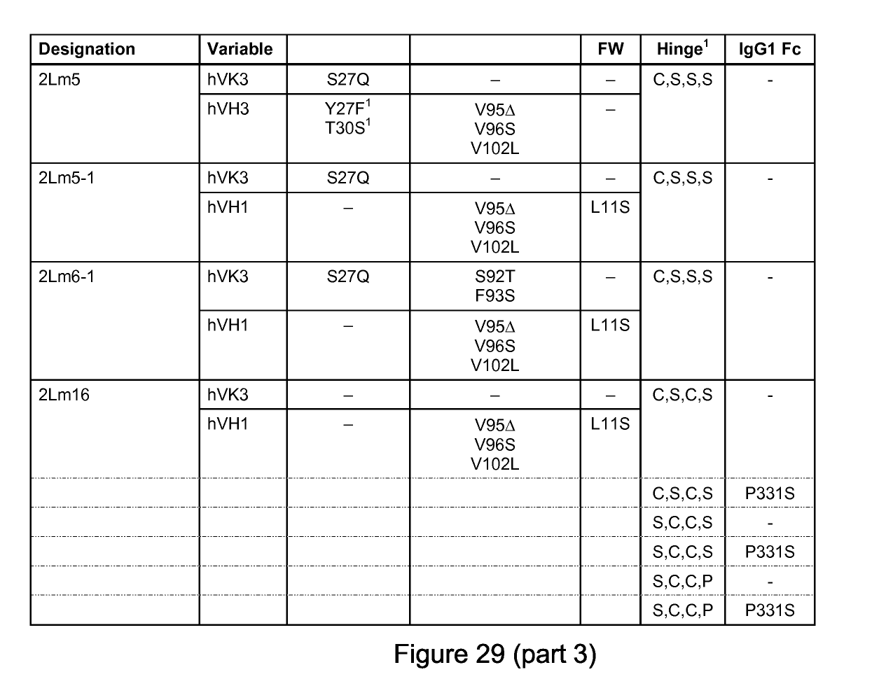

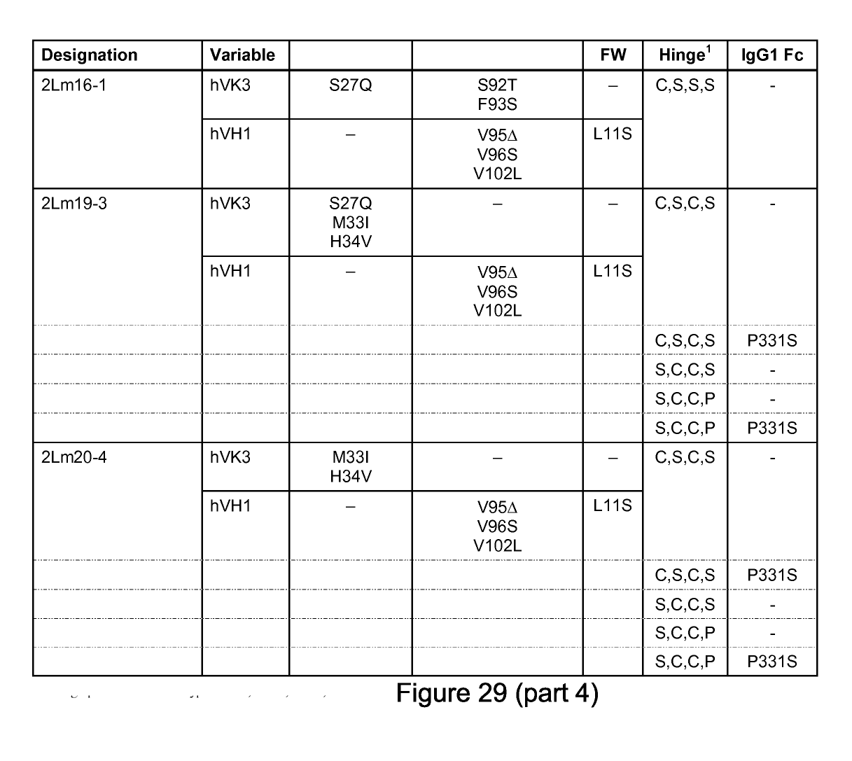

The ability of 018011 to inhibit the growth of various B-lymphoma cell lines was evaluated in vitro and was compared to that of RITUXAN® and TRU-015. Six human CD20+ B-cell lines were cultured for 96 hr with increasing concentrations of individual CD20-binders after which the viable cell number in each culture was enumerated. As shown in FIG. 27, neither 018011, TRU-015, nor RITUXAN® directly caused biologically meaningful growth inhibition in 5 out of the 6 B-lymphoma cell lines studied. The exception was the SU-DHL4 B-cell line whose growth was inhibited in a dose-dependent manner by TRU-015 and RITUXAN® but not significantly by 018011. Thus, the expression of CD20 is necessary but not sufficient to ensure the direct inhibition of BCL by any of the CD20-binders. Factors other than the degree of surface expression of CD20 govern the susceptibility of B-lymphoma cells to these anti-CD20 agents. Cross-linking 018011 or RITUXAN® on the surface of Ramos cells with anti-human IgG Fc enhanced the cytotoxic activity of both agents (FIG. 28).

All publications, patents and patent applications are herein incorporated by reference in their entirety to the same extent as if each individual publication, patent or patent application was specifically and individually indicated to be incorporated by reference in its entirety.

### Sequences

| | Construct Name | | |
|---|---|---|---|
| | VK3 | VH5 | |
| 18011 | 2Lm19-3 | 2H5m3 | EIVLTQSPATLSLSPGERATLSCRASQSVSYIV |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | SYYSNSYWYFDLWGRGTLVTVSS |
| | | | (SEQ ID NO: 1) |
| 18008 | 2Lm5 | 2H5 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGTGEVQLVQSGA |
| | | | EVKKPGESLKISCKGSGYSFTSYNMHWVRQMP |
| | | | GKGLEWMGAIYPGNGDTSYNQKFKGQVTISA |
| | | | DKSISTAYLQWSSLKASDTAMYYCAR |
| | | | VVYYSNSYWYFDLWGRGTLVTVSS |
| | | | (SEQ ID NO: 2) |
| 18010 | 2Lm19-3 | 2H5 | EIVLTQSPATLSLSPGERATLSCRASQSVSYIV |
| | | | WYQQKPGQAPRLLIYAPSNLASGIPARFSGSG |
| | | | SGTDFTLTISSLEPEDFAVYYCQQWSFNPPTF |
| | | | GQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | VVYYSNSYWYFDLWGRGTLVTVSS |
| | | | (SEQ ID NO: 3) |
| 18009 | 2Lm5 | 2H5m3 | EIVLTQSPATLSLSPGERATLSCRASQSVSYIV |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCA |
| | | | RVVYYSNSYWYFDLWGRGTLVTVSS |
| | | | (SEQ ID NO: 4) |
| 2Lm5 2H3m3 | 2Lm5 | 2H3m3 | EIVLTQSPATLSLSPGERATLSCRASQSVSSYMH |
| | | | WYQQKPGQAPRLLIYAPSNLASGIPARFSGSGS |
| | | | GTDFTLTISSLEPEDFAVYYCQQWSFNPPTFG |
| | | | QGTKVEIKDGGGSGGGGSGGGGTGEVQLLES |
| | | | GGGLVQPGGSLRLSCAASGFTFSSYNMHWVR |
| | | | QAPGKGLEWVSAIYPGNGDTSYNQKFKGRFT |
| | | | ISRDNSKNTLYLQMNSLRAEDTAVYYCA |
| | | | KSYYSNSYWYFDLWGRGTLVTVSS |
| | | | (SEQ ID NO: 5) |

| Construct Name | | |
|---|---|---|
| VK3 | VH1 | |
| 2L | 2Hm | EIVLTQSPATLSLSPGERATLSCRASSSVSSYMHW |
| | | YQQKPGQAPRLLIYAPSNLASGIPARFSGSGSGTD |
| | | FTLTISSLEPEDFAVYYCQQWSFNPPTFGQGTKV |
| | | EIKDGGGSGGGGSGGGGSSQVQLVQSGAEVKKP |
| | | GASVKVSCKASGYTFTSYNMHWVRQAPGQGLE |
| | | WMGAIYPGNGDTSYNQKFKGRVTMTRDTSTST |
| | | VYMELSSLRSEDTAVYYCARSVYYSN.YWYFDL |
| | | WGRGTLVTVSS |
| | | (SEQ ID NO: 6) |
| 2Lm | 2Hm | EIVLTQSPATLSLSPGERATLSCRASSSVSYMIW |
| | | YQQKPGQAPRLLIYAISNLASGIPARFSGSGSGT |
| | | DFTLTISSLEPEDFAVYYCQQWISNPPTFGQGTK |
| | | VEIKDGGGSGGGGSGGGGSSQVQLVQSGAEVK |
| | | KPGASVKVSCKASGYTFTSYNMHWVRQAPGQ |
| | | GLEWMGAIYPGNGDTSYNQKFKGRVTMTRDT |
| | | STSTVYMELSSLRSEDTAVYYCAR |
| | | SVYYSN.YWYFDLWGRGTLVTVSS |
| | | (SEQ ID NO: 7) |
| 2Lm | 2H | EIVLTQSPATLSLSPGERATLSCRASSSVSYMIW |
| | | YQQKPGQAPRLLIYAISNLASGIPARFSGSGSGT |
| | | DFTLTISSLEPEDFAVYYCQQWISNPPTFGQGTK |
| | | VEIKDGGGSGGGGSGGGGSSQVQLVQSGAEVK |
| | | KPGASVKVSCKASGYTFTSYNMHWVRQAPGQ |
| | | GLEWMGAIYPGNGDTSYNQKFKGRVTMTRDT |
| | | STSTVYMELSSLRSEDTAVYYCAR |
| | | VVYYSNSYWYFDLWGRGTLVTVSS |
| | | (SEQ ID NO: 8) |
| 2Lm1 | 2Hm | EIVLTQSPATLSLSPGERATLSCRASQSSVSYMH |
| | | WYQQKPGQAPRLLIYAPSNLASGIPARFSGSGS |
| | | GTDFTLTISSLEPEDFAVYYCQQWISNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTRD |
| | | TSTSTVYMELSSLRSEDTAVYYCAR |
| | | SVYYSN.YWYFDLWGRGTLVTVSS |
| | | (SEQ ID NO: 9) |
| 2Lm1 | 2H | EIVLTQSPATLSLSPGERATLSCRASQSSVSYMH |
| | | WYQQKPGQAPRLLIYAPSNLASGIPARFSGSGSG |
| | | TDFTLTISSLEPEDFAVYYCQQWISNPPTFGQGTK |
| | | VEIKDGGGSGGGGSGGGGSSQVQLVQSGAEVKKP |
| | | GASVKVSCKASGYTFTSYNMHWVRQAPGQGLEW |
| | | MGAIYPGNGDTSYNQKFKGRVTMTRDTSTSTVY |
| | | MELSSLRSEDTAVYYCARVVYYSNSYWYFDLW |
| | | GRGTLVTVSS |
| | | (SEQ ID NO: 10) |
| 2Lm2 | 2Hm | EIVLTQSPATLSLSPGERATLSCRASQSVSYMIW |
| | | YQQKPGQAPRLLIYAISNLASGIPARFSGSGSGT |
| | | DFTLTISSLEPEDFAVYYCQQWSFNPPTFGQGTK |
| | | VEIKDGGGSGGGGSGGGGSSQVQLVQSGAEVK |
| | | KPGASVKVSCKASGYTFTSYNMHWVRQA |
| | | PGQGLEWMGAIYPGNGDTSYNQKFKGRV |
| | | TMTRDTSTSTVYMELSSLRSEDTAVYYCA |
| | | RSVYYSN.YWYFDLWGRGTLVTVSS |
| | | (SEQ ID NO: 11) |
| 2Lm3 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWTSNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCA |
| | | RSVYYSN.YWYFDLWGRGTLVTVSS |
| | | (SEQ ID NO: 12) |
| 2Lm4 | 2Hm | EIVLTQSPATLSLSPGERATLSCRASQSVSSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYYSN.YWYFDLWGRGTLVTVSS |
| | | (SEQ ID NO: 13) |
| 2Lm5 | 2Hm | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGRV |
| | | TMTRDTSTSTVYMELSSLRSEDTAVYYCA |
| | | RSVYYSN.YWYFDLWGRGTLVTVSS |
| | | (SEQ ID NO: 14) |
| 2Lm5-1 | 2Hm3 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 15) |
| 2Lm5-2 | 2Hm4 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | V.YYSNSYWYFDLWGRGTLVTVSS |
| | | (SEQ ID NO: 16) |
| 2Lm5-3 | 2Hm5 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYY.NSYWYFDLWGRGTLVTVSS |
| | | (SEQ ID NO: 17) |
| 2Lm6 | 2Hm | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWTSNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCAR |
| | | SVYYSN.YWYFDLWGRGTLVTVSS |
| | | (SEQ ID NO: 18) |
| 2Lm6-1 | 2Hm3 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 19) |
| 2Lm6-2 | 2Hm4 | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWTSNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | V.YYSNSYWYFDLWGRGTLVTVSS |
| | | (SEQ ID NO: 20) |
| 2Lm6-3 | 2Hm5 | EIVLTQSPATLSLSPGERATLSCRASQSVSYMH |
| | | WYQQKPGQAPRLLIYAPSNLASGIPARFSGSG |
| | | SGTDFTLTISSLEPEDFAVYYCQQWTSNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYY.NSYWYFDLWGRGTLVTVSS |
| | | (SEQ ID NO: 21) |
| 2Lm7 | 2Hm | EIVLTQSPATLSLSPGERATLSCRASSSVSYMH |
| | | WYQQKPGQAPRLLIYATSNLASGIPARFSGSG |
| | | SGTDFTLTISSLEPEDFAVYYCQQWTSNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 22) |
| 2Lm8 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPYTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCA |
| | | RSVYYSN.YWYFDLWGRGTLVTVSS |
| | | (SEQ ID NO: 23) |
| 2Lm9 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPFTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 24) |
| 2Lm10 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPLTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCA |
| | | RSVYYSN.YWYFDLWGRGTLVTVSS |
| | | (SEQ ID NO: 25) |
| 2Lm11 | 2Hm | EIVLTQSPATLSLSPGERATLSCRASSSVSYMI |
| | | WYQQKPGQAPRLLIYAISNLASGIPARFSGSG |
| | | SGTDFTLTISSLEPEDFAVYYCQQWISNPITFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 26) |
| 2Lm12 | 2Hm | EIVLTQSPATLSLSPGERATLSCRASQSVSYMH |
| | | WYQQKPGQAPRLLIYATSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | SVYYSN.YWYFDLWGRGTLVTVSS |
| | | (SEQ ID NO: 27) |
| 2Lm13 | 2Hm | EIVLTQSPATLSLSPGERATLSCRASQSVSYMH |
| | | WYQQKPGQAPRLLIYAPSNLASGIPARFSGSGS |
| | | GTDFTLTISSLEPEDFAVYYCQQWISNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | SVYYSN.YWYFDLWGRGTLVTVSS |
| | | (SEQ ID NO: 28) |
| 2Lm14 | 2Hm | EIVLTQSPATLSLSPGERATLSCRASQSVSYMH |
| | | WYQQKPGQAPRLLIYATSNLASGIPARFSGSGS |
| | | GTDFTLTISSLEPEDFAVYYCQQWISNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | SVYYSN.YWYFDLWGRGTLVTVSS |
| | | (SEQ ID NO: 29) |
| 2Lm15 | 2Hm | EIVLTQSPATLSLSPGERATLSCRASQSVSYIHW |
| | | YQQKPGQAPRLLIYAPSNLASGIPARFSGSGSG |
| | | TDFTLTISSLEPEDFAVYYCQQWISNPPTFGQG |
| | | TKVEIKDGGGSGGGGSGGGGSSQVQLVQSGA |
| | | EVKKPGASVKVSCKASGYTFTSYNMHWVRQ |
| | | APGQGLEWMGAIYPGNGDTSYNQKFKGRVT |
| | | MTRDTSTSTVYMELSSLRSEDTAVYYCAR |
| | | SVYYSN.YWYFDLWGRGTLVTVSS |
| | | (SEQ ID NO: 30) |
| 2Lm16 | 2Hm3 | EIVLTQSPATLSLSPGERATLSCRASSSVSYMH |
| | | WYQQKPGQAPRLLIYAPSNLASGIPARFSGSG |
| | | SGTDFTLTISSLEPEDFAVYYCOQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 31) |
| 2Lm17-3 | 2Hm3 | EIVLTQSPATLSLSPGERATLSCRASQSVSYLS |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCA |
| | | S,YYSNSYWYFDLWGRGTLVTVSS |
| | | (SEQ ID NO: 32) |
| 2Lm17-4 | 2Hm3 | EIVLTQSPATLSLSPGERATLSCRASQSVSYLT |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 33) |
| 2Lm17-6 | 2Hm3 | EIVLTQSPATLSLSPGERATLSCRASQSVSYLY |
| | | WYQQKPGQAPRLLIYAPSNLASGIPARFSGSGS |
| | | GTDFTLTISSLEPEDFAVYYCQQWSFNPPTFGQ |
| | | GTKVEIKDGGGSGGGGSGGGGSSQVQLVQSGA |
| | | EVKKPGASVKVSCKASGYTFTSYNMHWVRQA |
| | | PGQGLEWMGAIYPGNGDTSYNQKFKGRVTMT |
| | | RDTSTSTVYMELSSLRSEDTAVYYCAR |
| | | S.YYSNSYWYFDLWGRGTLVTVSS |
| | | (SEQ ID NO: 34) |
| 2Lm17-8 | 2Hm3 | EIVLTQSPATLSLSPGERATLSCRASQSVSYLH |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 35) |
| 2Lm17- | 2Hm3 | EIVLTQSPATLSLSPGERATLSCRASQSVSYLN |
| 12 | | WYQQKPGQAPRLLIYAPSNLASGIPARFSGSG |
| | | SGTDFTLTISSLEPEDFAVYYCQQWSFNPPTF |
| | | GQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFK |
| | | GRVTMTRDTSTSTVYMELSSLRSEDTAVYYCA |
| | | RS.YYSNSYWYFDLWGRGTLVTVSS |
| | | (SEQ ID NO: 36) |
| 2Lm17- | 2Hm3 | EIVLTQSPATLSLSPGERATLSCRASQSVSYLA |
| 14 | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWVR |
| | | QAPGQGLEWMGAIYPGNGDTSYNQKFKGRVT |
| | | MTRDTSTSTVYMELSSLRSEDTAVYYCAR |
| | | S.YYSNSYWYFDLWGRGTLVTVSS |
| | | (SEQ ID NO: 37) |
| 2Lm18-2 | 2Hm3 | EIVLTQSPATLSLSPGERATLSCRASSSVSYLA |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 38) |
| 2Lm18-3 | 2Hm3 | EIVLTQSPATLSLSPGERATLSCRASSSVSYLN |
| | | WYQQKPGQAPRLLIYAPSNLASGIPARFSGS |
| | | GSGTDFTLTISSLEPEDFAVYYCQQWSFNPPT |
| | | FGQGTKVEIKDGGGSGGGGSGGGGSSQVQLV |
| | | QSGAEVKKPGASVKVSCKASGYTFTSYNMH |
| | | WVRQAPGQGLEWMGAIYPGNGDTSYNQKFKG |
| | | RVTMTRDTSTSTVYMELSSLRSEDTAVYYCAR |
| | | S.YYSNSYWYFDLWGRGTLVTVSS |
| | | (SEQ ID NO: 39) |
| 2Lm18-4 | 2Hm3 | EIVLTQSPATLSLSPGERATLSCRASSSVSYLD |
| | | WYQQKPGQAPRLLIYAPSNLASGIPARFSGSG |
| | | SGTDFTLTISSLEPEDFAVYYCQQWSFNPPTFG |
| | | QGTKVEIKDGGGSGGGGSGGGGSSQVQLVQS |
| | | GAEVKKPGASVKVSCKASGYTFTSYNMHWV |
| | | RQAPGQGLEWMGAIYPGNGDTSYNQKFKGR |
| | | VTMTRDTSTSTVYMELSSLRSEDTAVYYCAR |
| | | S.YYSNSYWYFDLWGRGTLVTVSS |
| | | (SEQ ID NO: 40) |
| 2Lm18-5 | 2Hm3 | EIVLTQSPATLSLSPGERATLSCRASSSVSYLSW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 41) |
| 2Lm18- | 2Hm3 | EIVLTQSPATLSLSPGERATLSCRASSSVSYLHW |
| 14 | | YQQKPGQAPRLLIYAPSNLASGIPARFSGSGSGT |
| | | DFTLTISSLEPEDFAVYYCQQWSFNPPTFGQGTK |
| | | VEIKDGGGSGGGGSGGGGSSQVQLVQSGAEVKK |
| | | PGASVKVSCKASGYTFTSYNMHWVRQAPGQGL |
| | | EWMGAIYPGNGDTSYNQKFKGRVTMTRDTSTST |
| | | VYMELSSLRSEDTAVYYCAR |
| | | S.YYSNSYWYFDLWGRGTLVTVSS |
| | | (SEQ ID NO: 42) |
| 2Lm19-1 | 2Hm3 | EIVLTQSPATLSLSPGERATLSCRASQSVSYIDW |
| | | YQQKPGQAPRLLIYAPSNLASGIPARFSGSGSGT |
| | | DFTLTISSLEPEDFAVYYCQQWSFNPPTFGQGTK |
| | | VEIKDGGGSGGGGSGGGGSSQVQLVQSGAEVK |
| | | KPGASVKVSCKASGYTFTSYNMHWVRQAPGQG |
| | | LEWMGAIYPGNGDTSYNQKFKGRVTMTRDTST |
| | | STVYMELSSLRSEDTAVYYCAR |
| | | S.YYSNSYWYFDLWGRGTLVTVSS |
| | | (SEQ ID NO: 43) |
| 2Lm19-2 | 2Hm3 | EIVLTQSPATLSLSPGERATLSCRASQSVSYISW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 44) |
| 2Lm19-3 | 2Hm3 | EIVLTQSPATLSLSPGERATLSCRASQSVSYIVW |
| | | YQQKPGQAPRLLIYAPSNLASGIPARFSGSGSGT |
| | | DFTLTISSLEPEDFAVYYCQQWSFNPPTFGQGT |
| | | KVEIKDGGGSGGGGSGGGGSSQVQLVQSGAEV |
| | | KKPGASVKVSCKASGYTFTSYNMHWVRQAPG |
| | | QGLEWMGAIYPGNGDTSYNQKFKGRVTMTRD |
| | | TSTSTVYMELSSLRSEDTAVYYCAR |
| | | S.YYSNSYWYFDLWGRGTLVTVSS |
| | | (SEQ ID NO: 45) |
| 2Lm19-4 | 2Hm3 | EIVLTQSPATLSLSPGERATLSCRASQSVSYIAW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 46) |
| 2Lm19-7 | 2Hm3 | EIVLTQSPATLSLSPGERATLSCRASQSVSYITW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 47) |
| 2Lm19-9 | 2Hm3 | EIVLTQSPATLSLSPGERATLSCRASQSVSYIIW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 48) |
| 2Lm19- | 2Hm3 | EIVLTQSPATLSLSPGERATLSCRASQSVSYIPW |
| 12 | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 49) |
| 2Lm19- | 2Hm3 | EIVLTQSPATLSLSPGERATLSCRASQSVSYINW |
| 14 | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 50) |
| 2Lm20-1 | 2Hm3 | EIVLTQSPATLSLSPGERATLSCRASSSVSYISW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 51) |
| 2Lm20-2 | 2Hm3 | EIVLTQSPATLSLSPGERATLSCRASSSVSYIAW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 52) |
| 2Lm20-4 | 2Hm3 | EIVLTQSPATLSLSPGERATLSCRASSSVSYIVW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 53) |
| 2Lm20-8 | 2Hm3 | EIVLTQSPATLSLSPGERATLSCRASSSVNYIYW |
| | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 54) |
| 2Lm20- | 2Hm3 | EIVLTQSPATLSLSPGERATLSCRASSSVSYIDW |
| 11 | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 55) |
| 2Lm20- | 2Hm3 | EIVLTQSPATLSLSPGERATLSCRASSSVSYIIW |
| 12 | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 56) |
| 2Lm20- | 2Hm3 | EIVLTQSPATLSLSPGERATLSCRASSSVSYIYW |
| 13 | | YQQKPGQAPRLLIYAPSNLASGIPARFSGSGSG |
| | | TDFTLTISSLEPEDFAVYYCQQWSFNPPTFGQG |
| | | TKVEIKDGGGSGGGGSGGGGSSQVQLVQSGAE |
| | | VKKPGASVKVSCKASGYTFTSYNMHWVRQAP |
| | | GQGLEWMGAIYPGNGDTSYNQKFKGRVTMTR |
| | | DTSTSTVYMELSSLRSEDTAVYYCAR |
| | | S.YYSNSYWYFDLWGRGTLVTVSS |
| | | (SEQ ID NO: 57) |
| 2Lm5 | 2H5m3 | EIVLTQSPATLSLSPGERATLSCRASQSVSYMH |
| (18009) | | WYQQKPGQAPRLLIYAPSNLASGIPARFSGS |
| | | GSGTDFTLTISSLEPEDFAVYYCQQWSFNPPT |
| | | FGQGTKVEIKDGGGSGGGGSGGGGTGEVQLV |
| | | QSGAEVKKPGESLKISCKGSGYSFTSYNMHW |
| | | VRQMPGKGLEWMGAIYPGNGDTSYNQKFKG |
| | | QVTISADKSISTAYLQWSSLKASDTAMYYCA |
| | | RVVYYSNSYWYFDLWGRGTLVTVSS |
| | | (SEQ ID NO: 58) |
| 2Lm5 | 2H3m3 | EIVLTQSPATLSLSPGERATLSCRASQSVSYMH |
| (2Lm5 | | WYQQKPGQAPRLLIYAPSNLASGIPARFSGSGS |
| 2H3m3) | | GTDFTLTISSLEPEDFAVYYCQQWSFNPPTFG |
| | | QGTKVEIKDGGGSGGGGSGGGGTGEVQLLES |
| | | GGGLVQPGGSLRLSCAASGFTFSSYNMHWVR |
| | | QAPGKGLEWVSAIYPGNGDTSYNQKFKGRFT |
| | | ISRDNSKNTLYLQMNSLRAEDTAVYYCA |
| | | KSYYSNSYWYFDLWGRGTLVTVSS |
| | | (SEQ ID NO: 59) |
| IgG1 | Hinge | DQEPKSCDKTHTSPPSS |
| | CSSS | (SEQ ID NO: 60) |
| IgG1 | CH2CH3 | APELLGGPSVFLFPPKPKDTLMISRTPEVTCVVVDVSHEDPEVKFN |
| | WT | WYVDGVEVHNAKTKPREEQYNSTYRVVSVLTVLHQDWLNGKE |
| | | YKCKVSNKALPAPIEKTISKAKGQPREPQVYTLPPSRDELTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPGK |
| | | (SEQ ID NO: 61) |
| IgG1 | CH2CH3 | APELLGGPSVFLFPPKPKDTLMISRTPEVTCVVVDVSHEDPEVKFN |
| | P331S | WYVDGVEVHNAKTKPREEQYNSTYRVVSVLTVLHQDWLNGKE |
| | | YKCKVSNKALPASIEKTISKAKGQPREPQVYTLPPSRDELTKNQV |
| | | SLTCLVKGFYPSDIAVEWESNGQPENNYKTTPPVLDSDGSFFLYS |
| | | KLTVDKSRWQQGNVFSCSVMHEALHNHYTQKSLSLSPGK |
| | | (SEQ ID NO: 62) |
| IgG1 | Hinge | DQEPKSCDKTHTCPPCP |
| | WT | (SEQ ID NO: 63) |
| IgG1 | Hinge | DQEPKSCDKTHTSPPCS |
| | CSCS | (SEQ ID NO: 64) |
| IgG1 | Hinge | DQEPKSSDKTHTCPPCS |
| | SCCS | (SEQ ID NO: 65) |
| IgG1 | Hinge | DQEPKSSDKTHTCPPCP |
| | SCCP | (SEQ ID NO: 66) |

Italics: Linker sequence
Underline: CDR sequences

### Nucleotide Sequences

| | |
|---|---|
| 2Lm1 | gaaattgtgttgacacagtctccagccaccctgtctttgtctccaggggaaagagccaccctctcctgca |
| | gggccagtcagagtgttagcagctacatgcactggtaccaacagaaacctggccaggctcctaggctcct |
| | catctatgccccatccaacctggcttctggaattccagccaggttcagtggcagtggatccgggacagac |
| | ttcactctcaccatcagcagcctagagcctgaagattttgcagtttattactgtcagcagtggatttcta |
| | accctcccacgttcggccaagggaccaaggtggaaatcaaa |
| | (SEQ ID NO: 67) |
| 2Lm2 | gaaattgtgttgacacagtctccagccaccctgtattgtctccaggggaaagagccaccctctcctgca |
| | gggccagtcagagtgttagctacatgatctggtaccaacagaaacctggccaggctcctaggctcctcat |
| | ctatgccatatccaacctggcttctggaattccagccaggttcagtggcagtggatccgggacagacttc |
| | actctcaccatcagcagcctagagcctgaagattttgcagtttattactgtcagcagtggagttttaacc |
| | ctcccacgttcggccaagggaccaaggtggaaatcaaa |
| | (SEQ ID NO: 68) |
| 2Lm3 | gaaattgtgttgacacagtccccagccaccccgtctttgtctccaggtgaaagagccaccctctcctgca |
| | gggccagttcaagtgttagctacatgatttggtaccaacagaaacctggccaggcacctaggctcctcat |
| | ctatgcaatctccaacctggcttctggaattccagccaggttcagtggcagtggatccgggacagacttc |
| | actctcaccatcagcagtctagagcctgaagattttgcagtttattactgtcagcagtggactagtaacc |
| | ctcccacgttcggccaagggaccaaggtggaaatcaaa |
| | (SEQ ID NO: 69) |
| 2Lm4 | gaaattgtgttgacacagtccccagccaccccgtctttgtctccaggtgaaagagccaccctctcctgca |
| | gggccagttcaagtgttagctcctacatgatttggtaccaacagaaacctggccaggcacctaggctcct |
| | catctatgcaatctccaacctggctctggaattccagccaggttcagtggcagtggatccgggacagac |
| | ttcactctcaccatcagcagtctagagcctgaagattttgcagtttattactgtcagcagtggactagta |
| | accctcccacgttcggccaagggaccaaggtggaaatcaaa |
| | (SEQ ID NO: 70) |
| 2Lm5 | gaaattgtgttgacacagtctccagccaccctgtctttgtctccaggggaaagagccaccctctcctgca |
| | gggccagtcagagtgttagctacatgcactggtaccaacagaaacctggccaggctcctaggctcctcat |
| | ctatgccccatccaacctggcttctggaattccagccaggttcagtggcagtggatccgggacagacttc |
| | actctcaccatcagcagcctagagcctgaagattttgcagtttattactgtcagcagtggagttttaacc |
| | ctcccacgttcggccaagggaccaaggtggaaatcaaa |
| | (SEQ ID NO: 71) |
| 2Lm5-1 | gaaattgtgttgacacagtctccagccaccctgtctttgtctccaggtgaaagagccaccctctcctgca |
| | gggecagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggagttttaacc |
| | ctcccacgttcggccaagggaccaaggtggaaatcaaa |
| | (SEQ ID NO: 72) |
| 2Lm5-2 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggagttttaacc |
| | ctcccacgttcggccaagggaccaaggtggaaatcaaa |
| | (SEQ ID NO: 73) |
| 2Lm5-3 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggagttttaacc |
| | ctcccacgttcggccaagggaccaaggtggaaatcaaa |
| | (SEQ ID NO: 74) |
| 2Lm6 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggacttctaacc |
| | ctcccacgttcggccaagggaccaaggtggaaatcaaa |
| | (SEQ ID NO: 75) |
| 2Lm6-1 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggactagtaac |
| | cctcccacgttcggccaagggaccaaggtggaaatcaaa |
| | (SEQ ID NO: 76) |
| 2Lm6-2 | gaaattgttgttgacacagtctccagccaccctgtctttgtctccaggtgaaagagccaccctctcctgca |
| | gggccagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggacttctaacc |
| | ctcccacgttcggccaagggaccaaggtggaaatcaaa |
| | (SEQ ID NO: 77) |
| 2Lm6-3 | Gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctc |
| | atctatgccccatccaacctggcttctggaattccagccaggttcagtggcagtggatccgggacagac |
| | ttcactctcaccatcagcagtctagagcctgaagattttgcagtttattactgtcagcagtggactagtaac |
| | cctcccacgttcggccaagggaccaaggtggaaatcaaa |
| | (SEQ ID NO: 78) |
| 2Lm7 | gaaattgtgttgacacagtctccagccaccctgtctttgtctccaggtgaaagagccaccctctcctgca |
| | gggccagttcaagtgttagctacatgcactggtaccaacagaaacctggccaggcacctaggctcctcat |
| | ctatgccacatccaacctggcttctggaattccagccaggttcagtggcagtggatccgggacagacttc |
| | actctcaccatcagcagtctagagcctgaagattttgcagtttattactgtcagcagtggactagtaacc |
| | ctcccacgttcggccaagggaccaaggtggaaatcaaa |
| | (SEQ ID NO: 79) |
| 2Lm8 | gaaattgtgttgacacagtctccagccaccctgtctttgtctccaggtgaaagagccaccctctcctgca |
| | gggccagttcaagtgttagctacatgatttggtaccaacagaaacctggccaggcacctaggctcctcat |
| | ctatgcaatctccaacctggcttctggaattccagccaggttcagtggcagtggatccgggacagacttc |
| | actctcaccatcagcagtctagagcctgaagattttgcagtttattactgtcagcagtggatttctaacc |
| | cttacactttcggccaagggaccaaggtggaaatcaaa |
| | (SEQ ID NO: 80) |
| 2Lm9 | gaaattgtgttgacacagtctccagccaccctgtctttgtctccaggtgaaagagccaccctctcctgca |
| | gggccagttcaagtgttagctacatgatttggtaccaacagaaacctggccaggcacctaggctcctcat |
| | ctatgcaatctccaacctggcttctggaattccagccaggttcagtggcagtggatccgggacagacttc |
| | actctcaccatcagcagtctagagcctgaagattttgcagtttattactgtcagcagtggatttctaacc |
| | ctttcactttcggccaagggaccaaggtggaaatcaaa |
| | (SEQ ID NO: 81) |
| 2Lm10 | gaaattgtgttgacacagtctccagccaccctgtctttgtctccaggtgaaagagccaccctctcctgca |
| | gggccagttcaagtgttagctacatgatttggtaccaacagaaacctggccaggcacctaggctcctcat |
| | ctatgcaatctccaacctggcttctggaattccagccaggttcagtggcagtggatccgggacagacttc |
| | actctcaccatcagcagtctagagcctgaagattttgcagtttattactgtcagcagtggatttctaacc |
| | ctctcactttcggccaagggaccaaggtggaaatcaaa |
| | (SEQ ID NO: 82) |
| 2Lm11 | gaaattgtgttgacacagtctccagccaccctgtctttgtctccaggtgaaagagccaccctctcctgca |
| | gggccagttcaagtgttagctacatgatttggtaccaacagaaacctggccaggcacctaggctcctca |
| | tctatgcaatctccaacctggcttctggaattccagccaggttcagtggcagtggatccgggacagactt |
| | cactctcaccatcagcagtctagagcctgaagattttgcagtttattactgtcagcagtggatttctaaccct |
| | atcactttcggccaagggaccaaggtggaaatcaaa |
| | (SEQ ID NO: 83) |
| 2Lm12 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctcat |
| | ctatgccacatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 84) |
| 2Lm13 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggatcagtaacc |
| | cacccacgttcggccaagggaccaaggtggaaatcaaa |
| | (SEQ ID NO: 85) |
| 2Lm14 | gaaattgtgttgacacagtctccagccaccctgtctttgtctccaggtgaaagagccaccctctcctgca |
| | gggccagtcagagtgttagctacatgcactggtaccaacagaaacctggccaggcacctaggctcctcat |
| | ctatgccacatccaacctggcttctggaattccagccaggttcagtggcagtggatccgggacagacttc |
| | actctcaccatcagcagtctagagcctgaagattttgcagtttattactgtcagcagtggatcagtaacc |
| | cacccacgttcggccaagggaccaaggtggaaatcaaa |
| | (SEQ ID NO: 86) |
| 2Lm15 | gaaattgtgttgacacagtctccagccaccctgtctttgtctccaggtgaaagagccaccctctcctgca |
| | gggccagtcagagtgttagctacatacactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggatcagtaacc |
| | cacccacgttcggccaagggaccaaggtggaaatcaaa |
| | (SEQ ID NO: 87) |
| 2Lm16 | gaaattgtgttgacacagtctccagccaccctgtctttgtctccaggtgaaagagccaccctctcctgca |
| | gggccagttccagtgttagctacatgcactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 88) |
| 2Lm17-3 | gaaattgtgttgacacagtctccagccaccctgtctttgtctccaggtgaaagagccaccctctcctgca |
| | gggccagtcagagtgttagctacctgtcttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 89) |
| 2Lm17-4 | gaaattgtgttgacacagtctccagccaccctgtctttgtctccaggtgaaagagccaccctctcctgca |
| | gggccagtcagagtgttagctacctgacttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 90) |
| 2Lm17-6 | gaaattgtgttgacacagtctccagccaccctgtctttgtctccaggtgaaagagccaccctctcctgca |
| | gggccagtcagagtgttagctacctgtactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 91) |
| 2Lm17-8 | gaaattgtgttgacacagtctccagccaccctgtctttgtctccaggtgaaagagccaccctctcctgca |
| | gggccagtcagagtgttagctacctgcactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 92) |
| 2Lm17-12 | gaaattgtgttgacacagtctccagccaccctgtctttgtctccaggtgaaagagccaccctctcctgca |
| | gggccagtcagagtgttagctacctgaactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 93) |
| 2Lm17-14 | gaaattgtgttgacacagtctccagccaccctgtctttgtctccaggtgaaagagccaccctctcctgca |
| | gggccagtcagagtgttagctacctggcttggtaccaacagaaacctggccaggcacctaggctcctcat |
| | ctatgccccatccaacctggatctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 94) |
| 2Lm18-2 | gaaattgtgttgacacagtctccagccaccctgtctttgtctccaggtgaaagagccaccctctcctgca |
| | gggccagttccagtgttagctacctggcctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 95) |
| 2Lm18-3 | gaaattgtgttgacacagtctccagccaccctgtctttgtctccaggtgaaagagccaccctctcctgca |
| | gggccagttccagtgttagctacctgaattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 96) |
| 2Lm18-4 | gaaattgtgttgacacagtctccagccaccctgtctttgtctccaggtgaaagagccaccctctcctgca |
| | gggccagttccagtgttagctacctggattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 97) |
| 2Lm18-5 | gaaattgtgttgacacagtctccagccaccctgtctttgtctccaggtgaaagagccaccctctcctgca |
| | gggccagttccagtgttagctacctgtcctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 98) |
| 2Lm18-14 | gaaattgtgttgacacagtctccagccaccctgtctttgtctccaggtgaaagagccaccctctcctgca |
| | gggccagttccagtgttagctacctgcattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 99) |
| 2Lm19-1 | gaaattgtgttgacacagtctccagccaccctgtctttgtctccaggtgaaagagccaccctctcctgca |
| | gggccagtcagagtgttagctacattgattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 100) |
| 2Lm19-2 | gaaattgtgttgacacagtctccagccaccctgtctttgtctccaggtgaaagagccaccctctcctgca |
| | gggccagtcagagtgttagctacatttcctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 101) |
| 2Lm19-3 | gaaattgtgttgacacagtctccagccaccctgtctttgtctccaggtgaaagagccaccctctcctgca |
| | gggccagtcagagtgttagctacattgtttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 102) |
| 2Lm19-4 | gaaattgtgttgacacagtctccagccaccctgtctttgtctccaggtgaaagagccaccctctcctgca |
| | gggccagtcagagtgttagctacattgcttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 103) |
| 2Lm19-7 | gaaattgtgttgacacagtctccagccaccctgtctttgtctccaggtgaaagagccaccctctcctgca |
| | gggccagtcagagtgttagctacattacctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 104) |
| 2Lm19-9 | gaaattgtgttgacacagtctccagccaccctgtctttgtctccaggtgaaagagccaccctctcctgca |
| | gggccagtcagagtgttagctacattatttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 105) |
| 2Lm19-12 | gaaattgtgttgacacagtctccagccaccctgtctttgtctccaggtgaaagagccaccctctcctgca |
| | gggccagtcagagtgttagctacattccctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 106) |
| 2Lm19-14 | gaaattgtgttgacacagtctccagccaccctgtctttgtctccaggtgaaagagccaccctctcctgca |
| | gggccagtcagagtgttagctacattaactggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 107) |
| 2Lm20-1 | gaaattgtgttgacacagtctccagccaccctgtctttgtctccaggtgaaagagccaccctctcctgca |
| | gggccagttccagtgttagctacatttcttggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 108) |
| 2Lm20-2 | gaaattgtgttgacacagtctccagccaccctgtctttgtctccaggtgaaagagccaccctctcctgca |
| | gggccagttccagtgttagctacattgcctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 109) |
| 2Lm20-4 | gaaattgtgttgacacagtctccagccaccctgtctttgtctccaggtgaaagagccaccctctcctgca |
| | gggccagttccagtgttagctacattgtttggtaccaacagaaacctggccaggcacctaggctcctcat |
| | ctatgccccatccaacctggcttctggaattccagccaggttcagtggcagtggatccgggacagactt |
| | cactctcaccatcagcagtctagagcctgaagattttgcagtttattactgtcagcagtggagttttaaccc |
| | tcccacgttcggccaagggaccaaggtggaaatcaaa |
| | (SEQ ID NO: 110) |
| 2Lm20-8 | gaaattgtgttgacacagtctccagccaccctgtctttgtctccaggtgaaagagccaccctctcctgca |
| | gggccagttccagtgttaactacatttattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgcagattttgcagtttattactgtcagcagtggagttttaacc |
| | ctcccacgttcggccaagggaccaaggtggaaatcaaa |
| | (SEQ ID NO: 111) |
| 2Lm19-11 | gaaattgtgttgacacagtctccagccaccctgtctttgtctccaggtgaaagagccaccctctcctgca |
| | gggccagttccagtgttagctacattgattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 112) |
| 2Lm20-12 | gaaattgtgttgacacagtctccagccaccctgtctttgtctccaggtgaaagagccaccctctcctgca |
| | gggccagttccagtgttagctccattatctggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 113) |
| 2Lm20-13 | gaaattgtgttgacacagtctccagccaccctgtctttgtctccaggtgaaagagccaccctctcctgca |
| | gggccagttccagtgttagctacatttattggtaccaacagaaacctggccaggcacctaggctcctcat |
| | ctatgccccatccaacctggcttctggaattccagccaggttcagtggcagtggatccgggacagacttc |
| | actctcaccatcagcagtctagagcctgaagattttgcagtttattactgtcagcagtggagttttaacc |
| | ctcccacgttcggccaagggaccaaggtggaaatcaaa |
| | (SEQ ID NO: 114) |
| 2Lm | gaaattgtgttgacacagtctccagccaccctgtctttgtctccaggggaaagagccaccctctcctgca |
| | gggccagtcagagtgttagctacatgatctggtaccaacagaaacctggccaggctcctaggctcctcat |
| | ctatgccatatccaacctggcttctggaattccagccaggttcagtggcagtggatccgggacagacttc |
| | actctcaccatcagcagcctagagcctgaagattttgcagtttattactgtcagcagtggatttctaacc |
| | ctcccacgttcggccaagggaccaaggtggaaatcaaa |
| | (SEQ ID NO: 115) |
| 2L | gaaattgtgttgacacagtctccagccaccctgtctttgtctccaggtgaaagagccaccctctcctgca |
| | gggccagtcagagtgttagcagctacatgcactggtaccaacagaaacctggccaggcacctaggct |
| | cctcatctatgccccatccaacctggcttctggaattccagccaggttcagtggcagtggatccgggac |
| | agacttcactctcaccatcagcagtctagagcctgaagattttgcagtttattactgtcagcagtggagtttt |
| | aaccctcccacgttcggccaagggaccaaggtggaaatcaaa |
| | (SEQ ID NO: 116) |
| 2H7_mVK | caggcttatctacagcagtctggggctgagctggtgaggcctggggcctcagtgaagatgtcctgcaagg |
| | cttctggctacacatttaccagttacaatatgcactgggtaaagcagacacctagacagggcctggaatg |
| | gattggagctatttatccaggaaatggtgatacttcctacaatcagaagttcaagggcaaggccacactg |
| | actgtagacaaatcctccagcacagcctacatgcagctcagcagcctgacatctgaagactctgcggtct |
| | atttctgtgcaagagtggtgtactatagtaactcttactggtacttcgatgtctggggcacagggaccac |
| | ggtcaccgtctct |
| | (SEQ ID NO: 117) |
| 2H5m3 | gaggtgcagctggtgcagtctggagcagaggtgaaaaagcccggagagtctctgaagatttcctgtaagg |
| | gatccggatacagctttaccagctacaacatgcactgggtgcgccagatgcccgggaaaggcctcgagtg |
| | gatgggggctatctatcctggaaatggtgatacatcctacaatcagaagttcaagggccaggtcactatc |
| | tcggccgacaagtccatcagcaccgcctacctgcaatggagcagcctgaaggcctcggacaccgccatgt |
| | attactgtgcacgcagcgtgtactatagtaactactggtacttcgatctctggggccgcggcaccctggt |
| | cactgtctcctct |
| | (SEQ ID NO: 118) |
| 2H5 | gaggtgcagctggtgcagtctggagcagaggtgaaaaagcccggagagtctctgaagatttcctgtaagg |
| | gatccggatacagctttaccagctacaacatgcactgggtgcgccagatgcccgggaaaggcctcgagtg |
| | gatgggggctatctatcctggaaatggtgatacatcctacaatcagaagttcaagggccaggtcactatc |
| | tcggccgacaagtccatcagcaccgcctacctgcaatggagcagcctgaaggcctcggacaccgccatgt |
| | attactgtgcacgcgtcgtgtactatagtaactcttactggtacttcgatctctggggccgcggcaccct |
| | ggtcactgtctcctct |
| | (SEQ ID NO: 119) |
| 2H3m3 | gaggtgcagctgttggagtctggtggaggcttggtacagcctggcggatccctgagactctcctgtgc |
| (mutant | agcctctggattcacctttagcagctataacatgcactgggtccgccaggctccagggaagggactgg |
| CDR3 in | agtgggtctcagctatctatcctggaaatggtgatacatcctacaatcagaagttcaagggccggttc |
| huVH3-47 | accatctccagagacaattccaagaacacgctgtatctgcaaatgaacagcctgagagccgaggacac |
| framework | ggccgtatattactgtgcgaaaagctactatagtaactcctactggtacttcgatctctggggccgcg |
| and JH2) | gcaccctggtcactgtctcctca |
| | (SEQ ID NO: 120) |
| 2Hm (CDR3 | caggtgcagctggtgcagtctggtgctgagagcaagaagcctggggcctcagtgaaggtttcctgcaa |
| mutant in | ggctagcggatacaccttcaccagctacaatatgcactgggtgcgacaggcgcctggacaagggctcg |
| huVH1 | agtggatgggagctatctatcctggaaatggtgatacatcctacaatcagaagttcaagggcagggtc |
| framework | accatgaccagagacacgtccacgagcacagtctacatggagctgagcagcctgagatctgaggacac |
| and JH2) | ggccgtgtattactgtgcccgaagcgtgtactatagtaactactggtacttcgatctctggggccgcg |
| | gcaccctggtcactgtctcctct |
| | (SEQ ID NO: 121) |
| 2H | caggtgcagctggtgcagtctggtgctgagagcaagaagcctggggcctcagtgaaggtttcctgcaa |
| (WT 2H7 | ggctagcggatacaccttcaccagctacaatatgcactgggtgcgacaggcgcctggacaagggctcg |
| CDRs in | agtggatgggagctatctatcctggaaatggtgatacatcctacaatcagaagttcaagggcagggtc |
| huVH1 | accatgaccagagacacgtccacgagcacagtctacatggagctgagcagcctgagatctgaggacac |
| framework | ggccgtgtatttctgtgcccgagtggtgtactatagtaactcttactggtacttcgatctctggggcc |
| and JH2) | gcggcaccctggtcactgtctcctct |
| | (SEQ ID NO: 122) |
| 2Hm3 | caggtgcagctggtgcagtctggtgctgagagcaagaagcctggggcctcagtgaaggtttcctgcaag |
| (CDR3 | gctagcggatacaccttcaccagctacaatatgcactgggtgcgacaggcgcctggacaagggctcgag |
| mutant) | tggatgggagctatctatcctggaaatggtgatacatcctacaatcagaagttcaagggcagggtcacc |
| | atgaccagagacacgtccacgagcacagtctacatggagctgagcagcctgagatctgaggacacggcc |
| | gtgtattactgtgcccgaagctactatagtaactcttactggtacttcgatctctggggccgcggcacc |
| | ctggtcactgtctcctct |
| | (SEQ ID NO: 123) |
| 2Hm4 | caggtgcagctggtgcagtctggtgctgagagcaagaagcctggggcctcagtgaaggtttcctgcaag |
| (CDR3 | gctagcggatacaccttcaccagctacaatatgcactgggtgcgacaggcgcctggacaagggctcgag |
| mutant) | tggatgggagctatctatcctggaaatggtgatacatcctacaatcagaagttcaagggcagggtcacc |
| | atgaccagagacacgtccacgagcacagtctacatggagctgagcagcctgagatctgaggacacggcc |
| | gtgtattactgtgcccgagtgtactatagtaactcttactggtacttcgatctctggggccgcggcacc |
| | ctggtcactgtctcctct |
| | (SEQ ID NO: 124) |
| 2Hm5 | caggtgcagctggtgcagtctggtgctgagagcaagaagcctggggcctcagtgaaggtttcctgcaag |
| (CDR3 | gctagcggatacaccttcaccagctacaatatgcactgggtgcgacaggcgcctggacaagggctcgag |
| mutant in | tggatgggagctatctatcctggaaatggtgatacatcctacaatcagaagttcaagggcagggtcacc |
| huVH1 | atgaccagagacacgtccacgagcacagtctacatggagctgagcagcctgagatctgaggacacggcc |
| framework | gtgtattactgtgcccgaagcgtgtactataactcttactggtacttcgatctctggggccgcggcacc |
| and JH2) | ctggtcactgtctcctct |
| | (SEQ ID NO: 125) |
| 2H3 | gaggtgcagctgttggagtctggtggaggcttggtacagcctggcggatccctgagactctcctgtacag |
| (WT 2H7 | cctctggattcacctttagcagctataacatgcactgggtccgccaggctccagggaagggactggagtg |
| CDRs in | ggtctcagctatctatcctggaaatggtgatacatcctacaatcagaagttcaagggccggttcaccatc |
| huVH3-47 | tccagagacaattccaagaacacgctgtatctgcaaatgaacagcctgagagccgaggacacggccgtat |
| framework | attactgtgcgaaagtcgtgtactatagtaactcctactggtacttcgatctctggggccgcggcaccct |
| and JH2) | ggtcactgtctcctct |
| | (SEQ ID NO: 126) |
| 2H7mVH | caggcttatctacagcagtctggggctgagctggtgaggcctggggcctcagtgaagatgtcctgcaagg |
| | cttctggctacacatttaccagttacaatatgcactgggtaaagcagacacctagacagggcctggaatg |
| | gattggagctatttatccaggaaatggtgatacttcctacaatcagaagttcaagggcaaggccacactg |
| | actgtagacaaatcctccagcacagcctacatgcagctcagcagcctgacatctgaagactctgcggtct |
| | atttctgtgcaagagtggtgtactatagtaactcttactggtacttcgatgtctggggcacagggaccac |
| | ggtcaccgtctct |
| | (SEQ ID NO: 127) |
| IgG1 | EPKSCDKTHTCPPCPAPELLGGPSVFLFPPKPKDTLMISRTPEVT |
| Hinge and | CVVVDVSHEDPEVKFNWYVDGVEVHNAKTKPREEQYNSTY |
| CH2CH3 | RVVSVLTVLHQDWLNGKEYKCKVSNKALPAPIEKTISKAK |
| | GQPREPQVYTLPPSRDELTKNQVSLTCLVKGFYPSDIAVE |
| | WESNGQPENNYKTTPPVLDSDGSFFLYSKLTVDKSRWQQG |
| | NVFSCSVMHEALHNHYTQKSLSLSPGK |
| | (SEQ ID NO: 128) |
| Human | MTTPRNSVNGTFPAEPMKGPIAMQSGPKPLFRRMSSLVGPTQSFF |
| CD20 | MRESKTLGAVQIMNGLFHIALGGLLMIPAGIYAPICVTVWYPLW |
| (Extracel | GGIMYIISGSLLAATEKNSRKCLVKGKMIMNSLSLFAAISGMILSI |
| lular | MDILNIKISHFLKMESLNFIRAHTPYINIYNCEPANPSEKNSPST |
| domain | QYCYSIQSLFLGILSVMLIFAFFQELVIAGIVENEWKRTCSRPKSNI |
| under- | VLLSAEEKKEQTIEIKEEVVGLTETSSQPKNEEDIEIIPIQEEEEEET |
| lined) | ETNFPEPPQDQESSPIENDSSP |
| | (SEQ ID NO: 129) |

What is claimed is:

1. A humanized CD20 binding molecule comprising an immunoglobulin heavy chain variable domain (VH) and an immunoglobulin light chain variable domain (VL) comprising framework regions and CDR1, CDR2 and CDR3 regions, wherein said framework regions are human immunoglobulin framework regions and wherein:
(a) the VH domain amino acid sequence comprises the amino acid sequence of a heavy chain CDR3 found in any one of SEQ ID NOS: 1-59; or
(b) the VL domain amino acid sequence comprises the amino acid sequence of a light chain CDR3 found in any one of SEQ ID NOS: 1-59; or
(c) the humanized CD20 binding molecule comprises a VH amino acid sequence of (a) and a VL amino acid sequence of (b); or
(d) the humanized CD20 binding molecule comprises a VH amino acid sequence of (a) and a VL amino acid sequence of (b) and wherein said VH and VL are found in the same reference sequence,
or a CD20 binding fragment of said binding molecule.

2. The humanized CD20 binding molecule of claim 1, further comprising:
(a) a VH domain amino acid sequence comprising an amino acid sequence that is at least 90% identical to the VH domain amino acid sequence found in any one of SEQ ID NOS: 1-59; or
(b) a VL domain amino acid sequence comprising an amino acid sequence that is at least 90% identical to the VL domain amino acid sequence found in any one of SEQ ID NOS: 1-59; or
(c) both a VH of (a) and a VL of (b); or
(d) the VH of (a) and the VL of (b), which are found in the same reference sequence.

3. The humanized CD20 binding molecule of claim 1, further comprising:
(a) a VH domain amino acid sequence comprising an amino acid sequence that is at least 95% identical to the VH domain amino acid sequence found in any one of SEQ ID NOS: 1-59; or
(b) a VL domain amino acid sequence comprising an amino acid sequence that is at least 95% identical to the VL domain amino acid sequence found in any one of SEQ ID NOS: 1-59; or
(c) both a VH of (a) and a VL of (b); or
(d) the VH of (a) and the VL of (b), which are found in the same reference sequence.

4. The humanized CD20 binding molecule of claim 1, further comprising:
(a) a VH domain amino acid sequence comprising the VH domain amino acid sequence found in any one of SEQ ID NOS: 1-59; or
(b) a VL domain amino acid sequence comprising the VL domain amino acid sequence found in any one of SEQ ID NOS: 1-59; or
(c) both a VH of (a) and a VL of (b); or
(d) the VH of (a) and the VL of (b), which are found in the same reference sequence.

5. The humanized CD20 binding molecule of any one of claims 1-4, which is an antibody or an antigen-binding fragment thereof.

6. The humanized CD20 binding molecule of any one of claims 1-4, which is a humanized Small Modular Immunopharmaceutical (SMIP).

7. A humanized SMIP that specifically binds CD20.

8. The humanized SMIP of claim 7 that binds the same epitope as, competes with or cross-competes with a humanized CD20 binding molecule comprising the amino acid sequence shown in any one of SEQ ID NOS: 1-59.

9. The humanized SMIP of any one of claims 6-8, comprising a hinge domain having the amino acid sequence shown in SEQ ID NO: 60.

10. The humanized SMIP of claim 9, comprising an effector domain comprising the amino acid sequence shown in SEQ ID NO: 61.

11. The humanized SMIP of claim 7, wherein the SMIP possesses at least one of the following properties:
(a) ADCC activity;
(b) CDC activity;
(c) reduces the growth of xenograft tumors;
(d) reduces the progression of disseminated lymphoma in vivo;
(e) depletes CD19+ B cells in peripheral blood, bone marrow and lymph nodes.

12. The humanized SMIP of claim 11, wherein the SMIP possesses at least three of the properties.

13. The CD20 binding molecule of any one of claims 1-6 or the humanized SMIP of any one of claims 7-12 which is detectably labeled.

14. A composition comprising the CD20 binding molecule of any one of claims 1-6 or the humanized SMIP of any one of claims 7-12.

15. A kit comprising the CD20 binding molecule of any one of claims 1-6, the humanized SMIP of any one of claims 7-12 or a composition of claim 14 and instructions for use.

16. A nucleic acid encoding the CD20 binding molecule or antigen-binding fragment of any one of claims 1-6 or the humanized SMIP of any one of claims 7-12.

17. The nucleic acid of claim 16 that encodes the VH domain and comprises a nucleotide sequence encoding the VH amino acid sequence found in any one of SEQ ID NOS: 1-59.

18. The nucleic acid of claim 17, comprising the nucleotide sequence of any one of SEQ ID NOS: 118-126.

19. The nucleic acid of claim 16 that encodes the VL domain and comprises a nucleotide sequence encoding the VL amino acid sequence found in any one of SEQ ID NOS: 1-59.

20. The nucleic acid of claim 19, comprising the nucleotide sequence of any one of SEQ ID NOS: 67-116.

21. The nucleic acid of any one of claims 16-20, operably linked to an expression control sequence.

22. A vector comprising the nucleic acid of any one of claims 16-21.

23. A host cell comprising the nucleic acid of any one of claims 16-21 or the vector of claim 22.

24. A method for producing a humanized CD20 binding molecule or antigen binding fragment thereof comprising the step of culturing the host cell of claim 23 and recovering the humanized CD20 binding molecule or antigen-binding fragment.

25. A method of detecting CD20 in a biological sample from a subject comprising the step of contacting the sample with the CD20 binding molecule or the humanized SMIP of claim 13 and detecting binding.

26. A method of treating a subject having or suspected of having a disease associated with aberrant B-cell activity, comprising administering to the subject a therapeutically effective amount of the humanized CD20 binding molecule of any one of claims 1-6, the humanized SMIP of any one of claims 7-12 or the composition of claim 14.

27. The method of claim 26, wherein the disease associated with aberrant B cell activity is rheumatoid arthritis, lupus erythamatosis or multiple sclerosis.

28. A method of reducing the number of B-cells in a subject in need thereof, comprising administering to the subject a therapeutically effective amount of the humanized CD20 binding molecule of any one of claims 1-6, the humanized SMIP of any one of claims 7-12 or the composition of claim 14.

29. The method of claim 28, wherein the subject is suffering from rheumatoid arthritis, lupus erythematosus or multiple sclerosis.


SEQUENCE LISTING (131 sequences)

SEQ ID NO: 1 - protein, 243 aa, Artificial Sequence
  source: /note=“Description of Artificial Sequence Synthetic construct”
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSS

SEQ ID NO: 2 - protein, 244 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 3 - protein, 244 aa, Artificial Sequence
  source: /note=“Description of Artificial Sequence Synthetic construct”
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 4 - protein, 244 aa, Artificial Sequence; identical to SEQ ID NO: 3
  source: /note=“Description of Artificial Sequence Synthetic construct”

SEQ ID NO: 5 - protein, 244 aa, Artificial Sequence
  source: /note=“Description of Artificial Sequence Synthetic construct”
EIVLTQSPATLSLSPGERATLSCRASQSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 6 - protein, 244 aa, Artificial Sequence
  source: /note=“Description of Artificial Sequence Synthetic construct”
EIVLTQSPATLSLSPGERATLSCRASSSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 7 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 8 - protein, 244 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 9 - protein, 244 aa, Artificial Sequence
  source: /note=“Description of Artificial Sequence Synthetic construct”
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 10 - protein, 245 aa, Artificial Sequence
  source: /note=“Description of Artificial Sequence Synthetic construct”
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 11 - protein, 243 aa, Artificial Sequence
  source: /note=“Description of Artificial Sequence Synthetic construct”
EIVLTQSPATLSLSPGERATLSCRASQSVSYMIWYQQKPGQAPRLLIYAISNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 12 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 13 - protein, 244 aa, Artificial Sequence
  source: /note=“Description of Artificial Sequence Synthetic construct”
EIVLTQSPATLSLSPGERATLSCRASQSVSSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 14 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 15 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 16 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 17 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 18 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 19 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 20 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 21 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 22 - protein, 243 aa, Artificial Sequence
  source: /note=“Description of Artificial Sequence Synthetic construct”
EIVLTQSPATLSLSPGERATLSCRASSSVSYMHWYQQKPGQAPRLLIYATSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 23 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPYTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 24 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPFTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 25 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPLTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 26 - protein, 243 aa, Artificial Sequence
  source: /note=“Description of Artificial Sequence Synthetic construct”
EIVLTQSPATLSLSPGERATLSCRASSSVSYMIWYQQKPGQAPRLLIYAISNLASGIPARFSGSGSGTDFTLTISSLEPEDFAVYYCQQWISNPITFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 27 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYATSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 28 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 29 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYATSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 30 - protein, 243 aa, Artificial Sequence
  source: /note=“Description of Artificial Sequence Synthetic construct”
EIVLTQSPATLSLSPGERATLSCRASQSVSYIHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 31 - protein, 243 aa, Artificial Sequence
  source: /note=“Description of Artificial Sequence Synthetic construct”
EIVLTQSPATLSLSPGERATLSCRASSSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 32 - protein, 242 aa, Artificial Sequence
  source: /note=“Description of Artificial Sequence Synthetic construct”
EIVLTQSPATLSLSPGERATLSCRASQSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCASYYSNSYWYFDLWGRGTLVTVSS

SEQ ID NO: 33 - protein, 243 aa, Artificial Sequence
  source: /note=“Description of Artificial Sequence Synthetic construct”
EIVLTQSPATLSLSPGERATLSCRASQSVSYLTWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 34 - protein, 243 aa, Artificial Sequence
  source: /note=“Description of Artificial Sequence Synthetic construct”
EIVLTQSPATLSLSPGERATLSCRASQSVSYLYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 35 - protein, 243 aa, Artificial Sequence
  source: /note=“Description of Artificial Sequence Synthetic construct”
EIVLTQSPATLSLSPGERATLSCRASQSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 36 - protein, 243 aa, Artificial Sequence
  source: /note=“Description of Artificial Sequence Synthetic construct”
EIVLTQSPATLSLSPGERATLSCRASQSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 37 - protein, 243 aa, Artificial Sequence
  source: /note=“Description of Artificial Sequence Synthetic construct”
EIVLTQSPATLSLSPGERATLSCRASQSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 38 - protein, 243 aa, Artificial Sequence
  source: /note=“Description of Artificial Sequence Synthetic construct”
EIVLTQSPATLSLSPGERATLSCRASSSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 39 - protein, 243 aa, Artificial Sequence
  source: /note=“Description of Artificial Sequence Synthetic construct”
EIVLTQSPATLSLSPGERATLSCRASSSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 40 - protein, 243 aa, Artificial Sequence
  source: /note=“Description of Artificial Sequence Synthetic construct”
EIVLTQSPATLSLSPGERATLSCRASSSVSYLDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 41 - protein, 243 aa, Artificial Sequence
  source: /note=“Description of Artificial Sequence Synthetic construct”
EIVLTQSPATLSLSPGERATLSCRASSSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 42 - protein, 243 aa, Artificial Sequence
  source: /note=“Description of Artificial Sequence Synthetic construct”
EIVLTQSPATLSLSPGERATLSCRASSSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 43 - protein, 243 aa, Artificial Sequence
  source: /note=“Description of Artificial Sequence Synthetic construct”
EIVLTQSPATLSLSPGERATLSCRASQSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 44 - protein, 243 aa, Artificial Sequence
  source: /note=“Description of Artificial Sequence Synthetic construct”
EIVLTQSPATLSLSPGERATLSCRASQSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 45 - protein, 243 aa, Artificial Sequence
  source: /note=“Description of Artificial Sequence Synthetic construct”
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 46 - protein, 243 aa, Artificial Sequence
  source: /note=“Description of Artificial Sequence Synthetic construct”
EIVLTQSPATLSLSPGERATLSCRASQSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 47 - protein, 243 aa, Artificial Sequence
  source: /note=“Description of Artificial Sequence Synthetic construct”
EIVLTQSPATLSLSPGERATLSCRASQSVSYITWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 48 - protein, 243 aa, Artificial Sequence
  source: /note=“Description of Artificial Sequence Synthetic construct”
EIVLTQSPATLSLSPGERATLSCRASQSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 49 - protein, 243 aa, Artificial Sequence
  source: /note=“Description of Artificial Sequence Synthetic construct”
EIVLTQSPATLSLSPGERATLSCRASQSVSYIPWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 50 - protein, 243 aa, Artificial Sequence
  source: /note=“Description of Artificial Sequence Synthetic construct”
EIVLTQSPATLSLSPGERATLSCRASQSVSYINWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 51 - protein, 243 aa, Artificial Sequence
  source: /note=“Description of Artificial Sequence Synthetic construct”
EIVLTQSPATLSLSPGERATLSCRASSSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 52 - protein, 243 aa, Artificial Sequence
  source: /note=“Description of Artificial Sequence Synthetic construct”
EIVLTQSPATLSLSPGERATLSCRASSSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 53 - protein, 243 aa, Artificial Sequence
  source: /note=“Description of Artificial Sequence Synthetic construct”
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 54 - protein, 243 aa, Artificial Sequence
  source: /note=“Description of Artificial Sequence Synthetic construct”
EIVLTQSPATLSLSPGERATLSCRASSSVNYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 55 - protein, 243 aa, Artificial Sequence
  source: /note=“Description of Artificial Sequence Synthetic construct”
EIVLTQSPATLSLSPGERATLSCRASSSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 56 - protein, 243 aa, Artificial Sequence
  source: /note=“Description of Artificial Sequence Synthetic construct”
EIVLTQSPATLSLSPGERATLSCRASSSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 57 - protein, 243 aa, Artificial Sequence
  source: /note=“Description of Artificial Sequence Synthetic construct”
EIVLTQSPATLSLSPGERATLSCRASSSVSYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 58 - protein, 244 aa, Artificial Sequence; identical to SEQ ID NO: 2
  source: /note=“Description of Artificial Sequence Synthetic construct”

SEQ ID NO: 59 - protein, 243 aa, Artificial Sequence
  source: /note=“Description of Artificial Sequence Synthetic construct”
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 60 - protein, 17 aa, Artificial Sequence
  source: /note=“Description of Artificial Sequence Synthetic construct”
DQEPKSCDKTHTSPPSS

SEQ ID NO: 61 - protein, 217 aa, Artificial Sequence
  source: /note=“Description of Artificial Sequence Synthetic construct”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 217 aa, Artificial Sequence
  source: /note=“Description of Artificial Sequence Synthetic construct”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 17 aa, Artificial Sequence
  source: /note=“Description of Artificial Sequence Synthetic construct”
DQEPKSCDKTHTCPPCP

SEQ ID NO: 64 - protein, 17 aa, Artificial Sequence
  source: /note=“Description of Artificial Sequence Synthetic construct”
DQEPKSCDKTHTSPPCS

SEQ ID NO: 65 - protein, 17 aa, Artificial Sequence
  source: /note=“Description of Artificial Sequence Synthetic construct”
DQEPKSSDKTHTCPPCS

SEQ ID NO: 66 - protein, 17 aa, Artificial Sequence
  source: /note=“Description of Artificial Sequence Synthetic construct”
DQEPKSSDKTHTCPPCP

SEQ ID NO: 67 - DNA, 321 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagcagctacatgcactggtaccaacagaaacctggccaggctcctaggctcctcatctatgccccatccaacctggcttctggaattccagccaggttcagtggcagtggatccgggacagacttcactctcaccatcagcagcctagagcctgaagattttgcagtttattactgtcagcagtggatttctaaccctcccacgttcggccaagggaccaaggtggaaatcaaa

SEQ ID NO: 68 - DNA, 318 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagctacatgatctggtaccaacagaaacctggccaggctcctaggctcctcatctatgccatatccaacctggcttctggaattccagccaggttcagtggcagtggatccgggacagacttcactctcaccatcagcagcctagagcctgaagattttgcagtttattactgtcagcagtggagttttaaccctcccacgttcggccaagggaccaaggtggaaatcaaa

SEQ ID NO: 69 - DNA, 318 bp, Artificial Sequence
  source: /note=“Description of Artificial Sequence Synthetic polynucleotide”
gaaattgtgttgacacagtccccagccaccccgtctttgtctccaggtgaaagagccaccctctcctgcagggccagttcaagtgttagctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggactagtaaccctcccacgttcggccaagggaccaaggtggaaatcaaa

SEQ ID NO: 70 - DNA, 321 bp, Artificial Sequence
  source: /note=“Description of Artificial Sequence Synthetic polynucleotide”
gaaattgtgttgacacagtccccagccaccccgtctttgtctccaggtgaaagagccaccctctcctgcagggccagttcaagtgttagctcctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggactagtaaccctcccacgttcggccaagggaccaaggtggaaatcaaa

SEQ ID NO: 71 - DNA, 318 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagctacatgcactggtaccaacagaaacctggccaggctcctaggctcctcatctatgccccatccaacctggcttctggaattccagccaggttcagtggcagtggatccgggacagacttcactctcaccatcagcagcctagagcctgaagattttgcagtttattactgtcagcagtggagttttaaccctcccacgttcggccaagggaccaaggtggaaatcaaa

SEQ ID NO: 72 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 73 - DNA, 318 bp, Artificial Sequence; identical to SEQ ID NO: 72
  source: /note=“Description of Artificial Sequence Synthetic polynucleotide”

SEQ ID NO: 74 - DNA, 318 bp, Artificial Sequence; identical to SEQ ID NO: 72
  source: /note=“Description of Artificial Sequence Synthetic polynucleotide”

SEQ ID NO: 75 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggacttctaaccctcccacgttcggccaagggaccaaggtggaaatcaaa

SEQ ID NO: 76 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggactagtaaccctcccacgttcggccaagggaccaaggtggaaatcaaa

SEQ ID NO: 77 - DNA, 318 bp, Artificial Sequence; identical to SEQ ID NO: 75
  source: /note=“Description of Artificial Sequence Synthetic polynucleotide”

SEQ ID NO: 78 - DNA, 318 bp, Artificial Sequence; identical to SEQ ID NO: 76
  source: /note=“Description of Artificial Sequence Synthetic polynucleotide”

SEQ ID NO: 79 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttcaagtgttagctacatgcactggtaccaacagaaacctggccaggcacctaggctcctcatctatgccacatccaacctggcttctggaattccagccaggttcagtggcagtggatccgggacagacttcactctcaccatcagcagtctagagcctgaagattttgcagtttattactgtcagcagtggactagtaaccctcccacgttcggccaagggaccaaggtggaaatcaaa

SEQ ID NO: 80 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttcaagtgttagctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggatttctaacccttacactttcggccaagggaccaaggtggaaatcaaa

SEQ ID NO: 81 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttcaagtgttagctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggatttctaaccctttcactttcggccaagggaccaaggtggaaatcaaa

SEQ ID NO: 82 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttcaagtgttagctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggatttctaaccctctcactttcggccaagggaccaaggtggaaatcaaa

SEQ ID NO: 83 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttcaagtgttagctacatgatttggtaccaacagaaacctggccaggcacctaggctcctcatctatgcaatctccaacctggcttctggaattccagccaggttcagtggcagtggatccgggacagacttcactctcaccatcagcagtctagagcctgaagattttgcagtttattactgtcagcagtggatttctaaccctatcactttcggccaagggaccaaggtggaaatcaaa

SEQ ID NO: 84 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccacatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 85 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggatcagtaacccacccacgttcggccaagggaccaaggtggaaatcaaa

SEQ ID NO: 86 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatgcactggtaccaacagaaacctggccaggcacctaggctcctcatctatgccacatccaacctggcttctggaattccagccaggttcagtggcagtggatccgggacagacttcactctcaccatcagcagtctagagcctgaagattttgcagtttattactgtcagcagtggatcagtaacccacccacgttcggccaagggaccaaggtggaaatcaaa

SEQ ID NO: 87 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatacactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggatcagtaacccacccacgttcggccaagggaccaaggtggaaatcaaa

SEQ ID NO: 88 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 89 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctgtcttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 90 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctgacttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 91 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctgtactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 92 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 93 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctgaactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 94 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacctggcttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 95 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacctggcctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 96 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacctgaattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 97 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacctggattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 98 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacctgtcctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 99 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacctgcattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 100 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattgattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 101 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacatttcctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 102 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattgtttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 103 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattgcttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 104 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattacctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 105 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattatttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 106 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattccctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 107 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagctacattaactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 108 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacatttcttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 109 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacattgcctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 110 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacattgtttggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 111 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttaactacatttattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 112 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacattgattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 113 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctccattatctggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 114 - DNA, 318 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagttccagtgttagctacatttattggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 115 - DNA, 318 bp, Artificial Sequence
  source: /note=“Description of Artificial Sequence Synthetic polynucleotide”
gaaattgtgttgacacagtctccagccaccctgtctttgtctccaggggaaagagccaccctctcctgcagggccagtcagagtgttagctacatgatctggtaccaacagaaacctggccaggctcctaggctcctcatctatgccatatccaacctggcttctggaattccagccaggttcagtggcagtggatccgggacagacttcactctcaccatcagcagcctagagcctgaagattttgcagtttattactgtcagcagtggatttctaaccctcccacgttcggccaagggaccaaggtggaaatcaaa

SEQ ID NO: 116 - DNA, 321 bp, Artificial Sequence
  source: /note=“Description of Artificial Sequence Synthetic polynucleotide”
gaaattgtgttgacacagtctccagccaccctgtctttgtctccaggtgaaagagccaccctctcctgcagggccagtcagagtgttagcagctacatgcactggtaccaacagaaacctggccaggcacctaggctcctcatctatgccccatccaacctggcttctggaattccagccaggttcagtggcagtggatccgggacagacttcactctcaccatcagcagtctagagcctgaagattttgcagtttattactgtcagcagtggagttttaaccctcccacgttcggccaagggaccaaggtggaaatcaaa

SEQ ID NO: 117 - DNA, 363 bp, Artificial Sequence
  source: /note=“Description of Artificial Sequence Synthetic polynucleotide”
caggcttatctacagcagtctggggctgagctggtgaggcctggggcctcagtgaagatgtcctgcaaggcttctggctacacatttaccagttacaatatgcactgggtaaagcagacacctagacagggcctggaatggattggagctatttatccaggaaatggtgatacttcctacaatcagaagttcaagggcaaggccacactgactgtagacaaatcctccagcacagcctacatgcagctcagcagcctgacatctgaagactctgcggtctatttctgtgcaagagtggtgtactatagtaactcttactggtacttcgatgtctggggcacagggaccacggtcaccgtctct

SEQ ID NO: 118 - DNA, 363 bp, Artificial Sequence
  source: /note=“Description of Artificial Sequence Synthetic polynucleotide”
gaggtgcagctggtgcagtctggagcagaggtgaaaaagcccggagagtctctgaagatttcctgtaagggatccggatacagctttaccagctacaacatgcactgggtgcgccagatgcccgggaaaggcctcgagtggatgggggctatctatcctggaaatggtgatacatcctacaatcagaagttcaagggccaggtcactatctcggccgacaagtccatcagcaccgcctacctgcaatggagcagcctgaaggcctcggacaccgccatgtattactgtgcacgcagcgtgtactatagtaactactggtacttcgatctctggggccgcggcaccctggtcactgtctcctct

SEQ ID NO: 119 - DNA, 366 bp, Artificial Sequence
  source: /note=“Description of Artificial Sequence Synthetic polynucleotide”
gaggtgcagctggtgcagtctggagcagaggtgaaaaagcccggagagtctctgaagatttcctgtaagggatccggatacagctttaccagctacaacatgcactgggtgcgccagatgcccgggaaaggcctcgagtggatgggggctatctatcctggaaatggtgatacatcctacaatcagaagttcaagggccaggtcactatctcggccgacaagtccatcagcaccgcctacctgcaatggagcagcctgaaggcctcggacaccgccatgtattactgtgcacgcgtcgtgtactatagtaactcttactggtacttcgatctctggggccgcggcaccctggtcactgtctcctct

SEQ ID NO: 120 - DNA, 363 bp, Artificial Sequence
  source: /note=“Description of Artificial Sequence Synthetic polynucleotide”
gaggtgcagctgttggagtctggtggaggcttggtacagcctggcggatccctgagactctcctgtgcagcctctggattcacctttagcagctataacatgcactgggtccgccaggctccagggaagggactggagtgggtctcagctatctatcctggaaatggtgatacatcctacaatcagaagttcaagggccggttcaccatctccagagacaattccaagaacacgctgtatctgcaaatgaacagcctgagagccgaggacacggccgtatattactgtgcgaaaagctactatagtaactcctactggtacttcgatctctggggccgcggcaccctggtcactgtctcctca

SEQ ID NO: 121 - DNA, 363 bp, Artificial Sequence
  source: /note=“Description of Artificial Sequence Synthetic polynucleotide”
caggtgcagctggtgcagtctggtgctgagagcaagaagcctggggcctcagtgaaggtttcctgcaaggctagcggatacaccttcaccagctacaatatgcactgggtgcgacaggcgcctggacaagggctcgagtggatgggagctatctatcctggaaatggtgatacatcctacaatcagaagttcaagggcagggtcaccatgaccagagacacgtccacgagcacagtctacatggagctgagcagcctgagatctgaggacacggccgtgtattactgtgcccgaagcgtgtactatagtaactactggtacttcgatctctggggccgcggcaccctggtcactgtctcctct

SEQ ID NO: 122 - DNA, 366 bp, Artificial Sequence
  source: /note=“Description of Artificial Sequence Synthetic polynucleotide”
caggtgcagctggtgcagtctggtgctgagagcaagaagcctggggcctcagtgaaggtttcctgcaaggctagcggatacaccttcaccagctacaatatgcactgggtgcgacaggcgcctggacaagggctcgagtggatgggagctatctatcctggaaatggtgatacatcctacaatcagaagttcaagggcagggtcaccatgaccagagacacgtccacgagcacagtctacatggagctgagcagcctgagatctgaggacacggccgtgtatttctgtgcccgagtggtgtactatagtaactcttactggtacttcgatctctggggccgcggcaccctggtcactgtctcctct

SEQ ID NO: 123 - DNA, 363 bp, Artificial Sequence
  source: /note=“Description of Artificial Sequence Synthetic polynucleotide”
caggtgcagctggtgcagtctggtgctgagagcaagaagcctggggcctcagtgaaggtttcctgcaaggctagcggatacaccttcaccagctacaatatgcactgggtgcgacaggcgcctggacaagggctcgagtggatgggagctatctatcctggaaatggtgatacatcctacaatcagaagttcaagggcagggtcaccatgaccagagacacgtccacgagcacagtctacatggagctgagcagcctgagatctgaggacacggccgtgtattactgtgcccgaagctactatagtaactcttactggtacttcgatctctggggccgcggcaccctggtcactgtctcctct

SEQ ID NO: 124 - DNA, 363 bp, Artificial Sequence
  source: /note=“Description of Artificial Sequence Synthetic polynucleotide”
caggtgcagctggtgcagtctggtgctgagagcaagaagcctggggcctcagtgaaggtttcctgcaaggctagcggatacaccttcaccagctacaatatgcactgggtgcgacaggcgcctggacaagggctcgagtggatgggagctatctatcctggaaatggtgatacatcctacaatcagaagttcaagggcagggtcaccatgaccagagacacgtccacgagcacagtctacatggagctgagcagcctgagatctgaggacacggccgtgtattactgtgcccgagtgtactatagtaactcttactggtacttcgatctctggggccgcggcaccctggtcactgtctcctct

SEQ ID NO: 125 - DNA, 363 bp, Artificial Sequence
  source: /note=“Description of Artificial Sequence Synthetic polynucleotide”
caggtgcagctggtgcagtctggtgctgagagcaagaagcctggggcctcagtgaaggtttcctgcaaggctagcggatacaccttcaccagctacaatatgcactgggtgcgacaggcgcctggacaagggctcgagtggatgggagctatctatcctggaaatggtgatacatcctacaatcagaagttcaagggcagggtcaccatgaccagagacacgtccacgagcacagtctacatggagctgagcagcctgagatctgaggacacggccgtgtattactgtgcccgaagcgtgtactataactcttactggtacttcgatctctggggccgcggcaccctggtcactgtctcctct

SEQ ID NO: 126 - DNA, 366 bp, Artificial Sequence
  source: /note=“Description of Artificial Sequence Synthetic polynucleotide”
gaggtgcagctgttggagtctggtggaggcttggtacagcctggcggatccctgagactctcctgtacagcctctggattcacctttagcagctataacatgcactgggtccgccaggctccagggaagggactggagtgggtctcagctatctatcctggaaatggtgatacatcctacaatcagaagttcaagggccggttcaccatctccagagacaattccaagaacacgctgtatctgcaaatgaacagcctgagagccgaggacacggccgtatattactgtgcgaaagtcgtgtactatagtaactcctactggtacttcgatctctggggccgcggcaccctggtcactgtctcctct

SEQ ID NO: 127 - DNA, 363 bp, Artificial Sequence; identical to SEQ ID NO: 117
  source: /note=“Description of Artificial Sequence Synthetic polynucleotide”

SEQ ID NO: 128 - protein, 232 aa, Artificial Sequence
  source: /note=“Description of Artificial Sequence Synthetic polypeptide”
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 130 - protein, 5 aa, Artificial Sequence
  source: /note=“Description of Artificial Sequence Synthetic peptide”
GGGGS

SEQ ID NO: 131 - protein, 25 aa, Artificial Sequence
  source: /note=“Description of Artificial Sequence Synthetic peptide”
GGGGSGGGGSGGGGSGGGGSGGGGS